(19) United States
(12) Patent Application Publication
(10) Pub. No.: US 2026/0133197 A1
(43) Pub. Date: May 14, 2026

(54) BIOMARKER BASED STANDARDS AND METHODS OF USE

(71) Applicant: Kite Pharma, Inc., Santa Monica, CA (US)

(72) Inventors: Hemamali J. Warshakoon, Lawndale, CA (US); Brandon M. Proctor, Los Angeles, CA (US)

(21) Appl. No.: 19/355,072

(22) Filed: Oct. 10, 2025

Related U.S. Application Data
(60) Provisional application No. 63/707,042, filed on Oct. 14, 2024.

(51) Int. Cl. G01N 33/569; G01N 33/96

(52) U.S. Cl. CPC G01N 33/56972; G01N 33/96; G01N 2496/10

(57) ABSTRACT

Provided herein are compositions and methods for fluorescence activated cell analysis of white blood cell populations. The compositions and methods enable thorough characterization each subpopulation of a greater cell population of leukocytes. Additionally, the biomarker-based standards and methods of are expedited as compared to the conventional technology and produce a positive control test material that reduces the cost, expedites the process, and improves the outcome of fluorescence activated cell analysis of blood cell populations.

### CROSS REFERENCE TO RELATED APPLICATIONS

This application claims priority to U.S. Provisional Application No. 63/707,042, filed Oct. 14, 2024, which is incorporated herein in its entirety for all purposes.

### TECHNICAL FIELD

The present disclosure relates to the field of cell therapy, and more specifically, compositions and methods for characterizing cell populations.

### BACKGROUND

T cell immunotherapy products derived via apheresis include a wide variety of cell types in addition to T cells. This heterogenous cell population includes NK cells, monocytes, B cells, early B progenitor cells, and stem cells, among others. Considered impurities, these cell populations must be identified and characterized at different stages of manufacturing processes for cell therapy products. However, it is challenging, and in many cases impossible, to find an effective positive control for characterizing and identifying these cell populations in combination with use of currently available analytic tools (e.g., flow cytometry systems). More specifically, existing positive controls do not include all markers necessary for characterizing a leukocyte population into subpopulations.

FIG. 1 (Prior Art) is flow cytometry data using a positive control, according to the prior art. In the lower right two panels, there are no representative cell populations for early progenitor cells (markers CD45dim/CD19+/CD10+) nor for stem cells (markers CD45dim/CD19+/CD34+). As such, existing technology does not allow for detection of early progenitor cells nor stem cells. Further, using a control according to the prior art does not allow accurate cell population characterization. More specifically, prior art does not enable quantification of all leukocyte subpopulations. This leads to errors when determining total leukocyte populations and subpopulation cross-comparison techniques are also not possible without a complete characterization. The technologies described herein address these unmet needs and additional unmet needs in the field. Additionally, existing positive controls are also inconsistent for a variety of reasons. One possible reason is due to differences in specimens used, thereby, leading to lot inconsistencies. The technologies described herein are synthetic in nature, thereby reducing lot to lot inconsistencies.

Thus, there is a need for the novel compositions, kits, and methods described herein to address many of the issues plaguing the cell therapy industry. Specifically, the technology described herein solves the problems described above and many more.

### SUMMARY

In various aspects, a composition for characterizing a population of cells into subpopulations is disclosed. In various embodiments, the composition for characterizing a population of cells into subpopulations comprises a population of biomarker-based standards. In various embodiments, each of the population of biomarker-based standards comprise a common marker. In various embodiments, the common marker corresponds to a marker associated with leukocytes. In various embodiments, each of the biomarker-based standards of the population of biomarker-based standards may comprise at least one of a plurality of marker sets. In various embodiments, the marker sets correspond to marker sets of T cells, NK cells, monocytes, B cells, early progenitor cells, and stem cells. In various embodiments, the population of biomarker-based standards may comprise a scaffold for restricting relative movement between the common marker and the at least one marker set.

In various embodiments, the common marker may comprise CD45, wherein CD45 may be comprised of populations of CD45hi and CD45dim.

In various embodiments, a marker set of the plurality of marker sets comprises CD10, CD19. CD45dim. In various embodiments, a marker set of the plurality of marker sets comprises CD19, CD34, and CD45dim. In various embodiments, a marker set of the plurality of marker sets comprises CD3, CD14, and CD45. In various embodiments, a marker set of the plurality of marker sets comprises CD3, CD45, and CD56. In various embodiments, a marker set of the plurality of marker sets comprises CD3, CD19, and CD45. In various embodiments, the plurality of marker sets comprise CD3, CD10, CD14, CD19, CD34, CD45, CD45dim, and CD56.

In various embodiments, at least some of the population of biomarker-based standards further comprise amine binding sites that react to Near IR viability dye. In various embodiments, between about 15-20% of the population of biomarker-based standards comprise amine binding sites. In various embodiments, between about 16.7-100% of the population of biomarker-based standards do not comprise amine binding sites.

In various embodiments, between about 50-60% of the population of biomarker-based standards express CD45hi. In various embodiments, between about 25.2-66.1% of the population of biomarker-based standards express CD45hi. In various embodiments, about 38.2% of the population of biomarker-based standards express CD45hi.

In various embodiments, between about 40-50% of the population of biomarker-based standards express CD45dim. In various embodiments, between about 32.3-74.7% of the population of biomarker-based standards express CD45dim. In various embodiments, about 59.7% of the population of biomarker-based standards express CD45dim.

In various embodiments, between about 50-60% of the population of biomarker-based standards comprise CD10, CD19, and CD45dim. In various embodiments, between about 29.7-66.7% of the population of biomarker-based standards comprise CD10, CD19, and CD45dim. In various embodiments, about 49.2 of the population of biomarker-based standards express CD10, CD19, and CD45dim.

In various embodiments, between about 50-60% of the population of biomarker-based standards comprise CD19, CD34, and CD45dim. In various embodiments, between about 0.3-68.2% of the population of biomarker-based standards comprise CD19, CD34, and CD45dim. In various embodiments, about 45.6% of the population of biomarker-based standards express CD19, CD34, and CD45dim.

In various embodiments, the markers may be embedded within the scaffolds. In various embodiments, the markers may be located on a surface of the scaffolds. In various embodiments, the scaffold may comprise a phospholipid bilayer. In various embodiments, the scaffold may comprise a hydrogel, an aerogel, a cryogel, an organogel, a nanogel, a conductive hydrogel, or any combination thereof.

In various aspects, a kit for characterizing a population of cells into subpopulations is disclosed. In various embodiments, the kit for characterizing a population of cells into subpopulations comprises a population of biomarker-based standards. In various embodiments, each biomarker-based standard comprises a common marker. In various embodiments, the common marker corresponds to a marker associated with leukocytes. In various embodiments, each biomarker-based standard comprises at least one of a plurality of marker sets. In various embodiments, the marker sets correspond to marker sets of T cells, NK cells, monocytes, B cells, early progenitor cells, and stem cells. In various embodiments, each of the biomarker-based standard comprises a scaffold for restricting relative movement between the common marker and the at least one marker set. In various embodiments, the kit for characterizing a population of cells into subpopulations comprises an antibody cocktail.

In various embodiments, the common marker comprises CD45. In various embodiments, CD45 may be comprised of populations of CD45hi and CD45dim. In various embodiments, a marker set of the plurality of marker sets comprises CD10, CD19, CD45dim. In various embodiments, a marker set of the plurality of marker sets comprises CD19. CD34, and CD45dim. In various embodiments, a marker set of the plurality of marker sets comprises CD3, CD14, and CD45. In various embodiments, a marker set of the plurality of marker sets comprises CD3, CD45, and CD56. In various embodiments, a marker set of the plurality of marker sets comprises CD3, CD19, and CD45. In various embodiments, the plurality of marker sets comprise CD3, CD10, CD14, CD19, CD34, CD45, CD45dim, and CD56.

In various embodiments, at least some of the population of biomarker-based standards further comprise amine binding sites that react to Near IR viability dye. In various embodiments, between about 15-20% of the population of biomarker-based standards comprise amine binding sites. In various embodiments, between about 16.7-100% of the population of biomarker-based standards do not comprise amine binding sites.

In various embodiments, between about 50-60% of the population of biomarker-based standards express CD45hi. In various embodiments, between about 25.2-66.1% of the population of biomarker-based standards express CD45hi. In various embodiments, about 38.2% of the population of biomarker-based standards express CD45hi.

In various embodiments, between about 40-50% of the population of biomarker-based standards express CD45dim. In various embodiments, between about 32.3-74.7% of the population of biomarker-based standards express CD45dim. In various embodiments, about 59.7% of the population of biomarker-based standards express CD45dim.

In various embodiments, between about 50-60% of the population of biomarker-based standards comprise CD10, CD19, and CD45dim. In various embodiments, between about 29.7-66.7% of the population of biomarker-based standards comprise CD10, CD19, and CD45dim. In various embodiments, about 49.2 of the population of biomarker-based standards express CD10, CD19, and CD45dim.

In various embodiments, between about 50-60% of the population of biomarker-based standards comprise CD19, CD34, and CD45dim. In various embodiments, between about 0.3-68.2% of the population of biomarker-based standards comprise CD19, CD34, and CD45dim. In various embodiments, about 45.6% of the population of biomarker-based standards express CD19. CD34, and CD45dim.

In various embodiments, the markers may be embedded within the scaffolds. In various embodiments, the markers may be located on a surface of the scaffolds. In various embodiments, the scaffold comprises a phospholipid bilayer. In various embodiments, the scaffold comprise a hydrogel, an aerogel, a cryogel, an organogel, a nanogel, a conductive hydrogel, or any combination thereof.

In various embodiments, the antibody cocktail comprises an antibody for detecting the CD3. In various embodiments, the antibody cocktail comprises an antibody for detecting the CD10. In various embodiments, the antibody cocktail comprises an antibody for detecting the CD14. In various embodiments, the antibody cocktail comprises an antibody for detecting the CD19. In various embodiments, the antibody cocktail comprises an antibody for detecting the CD34. In various embodiments, the antibody cocktail comprises an antibody for detecting the CD45/CD45dim. In various embodiments, the antibody cocktail comprises an antibody for detecting the CD56.

In various embodiments, the antibody for detecting the CD3 may be conjugated to an APC fluorochrome. In various embodiments, the antibody for detecting the CD10 marker may be conjugated to a FITC fluorochrome. In various embodiments, the antibody for detecting the CD14 marker may be conjugated to a PerCP Cy5.5 fluorochrome. In various embodiments, the antibody for detecting the CD19 may be conjugated to a PE.Cy7 fluorochrome. In various embodiments, the antibody for detecting the CD34 may be conjugated to a BV421 fluorochrome. In various embodiments, the antibody for detecting the CD45/CD45dim marker may be conjugated to a V500 fluorochrome. In various embodiments, the antibody for detecting the CD56 marker may be conjugated to a PE fluorochrome.

In various aspects, a method for characterizing a population of cells into subpopulations is disclosed. In various embodiments, the method comprises providing a biological sample comprising a population of leukocytes. In various embodiments, the population of leukocytes comprises a plurality of subpopulations, wherein the subpopulations comprise T cells, NK cells, monocytes, B cells, B progenitor cells, and stem cells. In various embodiments, the method comprises providing a known quantity of a population of biomarker-based standards. In various embodiments, the biomarker-based standards comprise a common marker. In various embodiments, the common marker corresponds to a marker associated with leukocytes. In various embodiments, the biomarker-based standards comprise at least one of a plurality of marker sets. In various embodiments, the marker sets correspond to marker sets of T cells, NK cells, monocytes, B cells, early progenitor cells, and stem cells. In various embodiments, the biomarker-based standards may comprise a scaffold for restricting relative movement between the common marker and the at least one marker set. In various embodiments, the method may comprise contacting the population of leukocytes and the population of biomarker-based standards with an antibody cocktail to create a mixture. In various embodiments, the method may comprise analyzing the mixture with an analytical device. In various embodiments, the method may comprise characterizing the population of cells by subpopulation using the biomarker-based standards as a reference.

In various embodiments, the step of characterizing comprises determining a quantity of the population of leukocytes and quantities for each of the subpopulations.

In various embodiments, the step of characterizing comprises determining relative quantities or percentages of each of the subpopulations and the population of leukocytes.

In various embodiments, the common marker may comprise CD45. In various embodiments, CD45 may be comprised of populations of CD45hi and CD45dim.

In various embodiments, a marker set of the plurality of marker sets comprises CD10, CD19. CD45dim. In various embodiments, a marker set of the plurality of marker sets comprises CD19, CD34, and CD45dim. In various embodiments, a marker set of the plurality of marker sets comprises CD3, CD14, and CD45. In various embodiments, a marker set of the plurality of marker sets comprises CD3, CD45, and CD56. In various embodiments, a marker set of the plurality of marker sets comprises CD3. CD19, and CD45. In various embodiments, the plurality of marker sets comprise CD3, CD10, CD14, CD19, CD34, CD45, CD45dim, and CD56.

In various embodiments, at least some of the population of biomarker-based standards further comprise amine binding sites that react to Near IR viability dye. In various embodiments, between about 15-20% of the population of biomarker-based standards comprise amine binding sites. In various embodiments, between about 16.7-100% of the population of biomarker-based standards do not comprise amine binding sites.

In various embodiments, between about 50-60% of the population of biomarker-based standards express CD45hi. In various embodiments, between about 25.2-66.1% of the population of biomarker-based standards express CD45hi. In various embodiments, about 38.2% of the population of biomarker-based standards express CD45hi.

In various embodiments, between about 40-50% of the population of biomarker-based standards express CD45dim. In various embodiments, between about 32.3-74.7% of the population of biomarker-based standards express CD45dim. In various embodiments, about 59.7% of the population of biomarker-based standards express CD45dim.

In various embodiments, between about 50-60% of the population of biomarker-based standards comprise CD10, CD19, and CD45dim. In various embodiments, between about 29.7-66.7% of the population of biomarker-based standards comprise CD10, CD19, and CD45dim. In various embodiments, about 49.2 of the population of biomarker-based standards express CD10, CD19, and CD45dim.

In various embodiments, between about 50-60% of the population of biomarker-based standards comprise CD19, CD34, and CD45dim. In various embodiments, between about 0.3-68.2% of the population of biomarker-based standards comprise CD19, CD34, and CD45dim. In various embodiments, about 45.6% of the population of biomarker-based standards express CD19, CD34, and CD45dim.

In various embodiments, the markers may be embedded within the scaffolds. In various embodiments, the markers may be located on a surface of the scaffolds. In various embodiments, the scaffold comprises a phospholipid bilayer. In various embodiments, the scaffold comprises a hydrogel, an aerogel, a cryogel, an organogel, a nanogel, a conductive hydrogel, or any combination thereof.

In various embodiments, the method further comprises use of an antibody cocktail, wherein the antibody cocktail comprises an antibody for detecting the CD3, an antibody for detecting the CD10, an antibody for detecting the CD14, an antibody for detecting the CD19, an antibody for detecting the CD34, an antibody for detecting the CD45hi/CD45dim, and an antibody for detecting the CD56.

In various embodiments, the antibody for detecting the CD3 may be conjugated to an APC fluorochrome. In various embodiments, the antibody for detecting the CD10 marker may be conjugated to a FITC fluorochrome. In various embodiments, the antibody for detecting the CD14 marker may be conjugated to a PerCP Cy5.5 fluorochrome. In various embodiments, the antibody for detecting the CD19 may be conjugated a PE.Cy7 fluorochrome. In various embodiments, the antibody for detecting the CD34 may be conjugated to a BV421 fluorochrome. In various embodiments, the antibody for detecting the CD45/CD45dim marker may be conjugated to a V500 fluorochrome. In various embodiments, the antibody for detecting the CD56 marker may be conjugated to a PE fluorochrome.

In various embodiments, the biological sample comprises an apheresis. In various embodiments, the biological sample comprises a final product.

In various embodiments, the analytical device comprises a flow cytometer. In various embodiments, the flow cytometer comprises a plurality of lasers directed through a flow channel for exciting the fluorochromes. In various embodiments, the flow cytometry comprises at least one detector.

In various embodiments, the step of analyzing further comprises flowing the mixture through the flow cell. In various embodiments, the step of analyzing further comprises interrogating each of the cells in the populations of leukocytes and each of the biomarker-based standards with each of the plurality of lasers. In various embodiments, the step of analyzing further comprises capturing signal intensity data for each marker.

### BRIEF DESCRIPTION OF THE FIGURES

FIG. 1 (Prior Art) is flow cytometry data using a positive control, according to the prior art.

FIG. 2 is a cartoon illustration of a biomarker-based standard, according to various embodiments.

FIG. 3 is a cartoon illustration of labeled detection molecule, according to various embodiments.

FIG. 4 is a cartoon illustration of a labeled detection molecule bound to a marker associated with a scaffold of a biomarker-based standard, according to various embodiments.

FIG. 5 illustrates a process for characterizing a population of cells into subpopulations using biomarker-based standards, according to various embodiments.

FIG. 6 is a schematic diagram of flow cytometer for carrying out methods using biomarker-based standards, according to various embodiments.

FIG. 7 is a schematic diagram of a computer system in accordance with various embodiments.

FIG. 8 is a chart showing markers that relate to identifying a subpopulation of cells, according to various embodiments.

FIG. 9 illustrates an exemplary leukocyte population, according to various embodiments.

FIG. 10 is experimental flow cytometry data from diseased biological samples showing the percentage of leukocyte populations expressing CD45hi marker.

FIG. 11 is experimental flow cytometry data from diseased biological samples showing the percentage of leukocyte populations expressing CD45dim marker.

FIG. 12 is experimental flow cytometry data from diseased biological samples showing the percentage of leukocyte populations expressing CD45dim, CD19+, and CD10+.

FIG. 13 is experimental flow cytometry data from diseased biological samples showing the percentage of leukocyte populations expressing CD45dim, CD19+, and CD34+.

FIG. 14 is experimental flow cytometry data from healthy biological samples showing the percentage of leukocyte populations expressing CD45hi marker.

FIG. 15 is experimental flow cytometry data from healthy biological samples showing the percentage of leukocyte populations expressing CD45dim marker.

FIG. 16 is experimental flow cytometry data from healthy biological samples showing the percentage of leukocyte populations expressing CD45hi and CD56+ markers and lacking CD14 and CD3.

FIG. 17 is experimental flow cytometry data from healthy biological samples showing the percentage of leukocyte populations expressing CD45hi and CD3+ markers lacking CD14 and CD56.

FIG. 18 is experimental flow cytometry data from healthy biological samples showing the percentage of leukocyte populations expressing CD45hi and 19+ markers while lacking CD14 and CD3.

FIG. 19 is experimental flow cytometry data from healthy biological samples showing the percentage of leukocyte populations expressing CD45dim, CD19+, and CD34+.

FIG. 20 is experimental flow cytometry data from healthy biological samples showing the percentage of leukocyte populations expressing CD45dim, CD19+, and CD10+.

FIG. 21 is experimental flow cytometry data using a biomarker-based standard.

### DETAILED DESCRIPTION OF CERTAIN EMBODIMENTS

The present disclosure, among other things, provides insights and technologies useful in the cell therapy industry and more specifically in T cell and/or CAR T cell manufacturing. More specifically, the embodiment described herein may be useful in design and manufacture of cell therapy products. Among other things, the technology described herein enables more consistent cell therapy products (e.g., cell therapy products meeting or exciting quality standards).

In the figures, specification, claims, and abstract numerous specific details are set forth to provide a thorough understanding of the various embodiments. A skilled artisan will appreciate that the systems and methods described herein may be used in a variety of ways and circumstances that are not limited to what is specifically detailed herein. Additionally, the skilled artisan will appreciate that certain embodiments may be practiced without these specific details. Furthermore, one skilled in the art can readily appreciate that the specific sequences in which methods are presented and performed are illustrative and it is contemplated that the sequences can be varied and still remain within the spirit and scope of certain embodiments.

While the present teachings are described in conjunction with various embodiments, it is not intended that the present teachings be limited to such embodiments. On the contrary, the present teachings encompass various alternatives, modifications, and equivalents, as will be appreciated by those skilled in the art.

### I. Definitions

Except as otherwise expressly provided herein, each of the following terms shall have the meaning set forth below. Additional definitions are set forth throughout the application. Unless defined otherwise, all technical and scientific terms used herein have the meaning as commonly understood by one of ordinary skill in the art. For example, the manual Current Protocols In Immunology, edited by John E. Coligan, Ada M. Kruisbeek, David H. Margulies, Ethan M. Shevach, Warren Strober, (Series Editor: Richard Coico), ISBN 0471522767; Concise Dictionary of Biomedicine and Molecular Biology, Juo, Pei-Show, 2nd ed., 2002, CRC Press; The Dictionary of Cell and Molecular Biology, 3rd ed., 1999, Academic Press; and the Oxford Dictionary of Biochemistry and Molecular Biology, Revised, 2006, Oxford University Press, provide one of skill with a general dictionary of many of the terms used in this application.

Units, prefixes, and symbols are denoted in their Systeme International de Unites (SI) accepted form. Numeric ranges are inclusive of the numbers defining the range. The disclosure provided herein are not limitations of the various aspects of the application, which may be by reference to the specification as a whole. Unless defined otherwise, all technical and scientific terms used herein have the same meaning as commonly understood by one of ordinary skill in the art to which this disclosure is related. For example, Juo, “The Concise Dictionary of Biomedicine and Molecular Biology”, 2nd ed., (2001), CRC Press; “The Dictionary of Cell & Molecular Biology”, 5th ed., (2013), Academic Press; and “The Oxford Dictionary Of Biochemistry And Molecular Biology”, Cammack et al. eds., 2nd ed, (2006), Oxford University Press, provide those of skill in the art with a general dictionary for many of the terms used in this disclosure.

As described herein, any concentration range, percentage range, ratio range or integer range is to be understood to be inclusive of the value of any integer within the recited range and, when appropriate, fractions thereof (such as one-tenth and one-hundredth of an integer), unless otherwise indicated.

As referred to herein, the terms “about” or “comprising essentially of” may refer to a value or composition that is within an acceptable error range for certain value or composition as determined by one of ordinary skill in the art, which will depend in part on how the value or composition is measured or determined, i.e., the limitations of the measurement system. For example, “about” or “comprising essentially of” may mean within 1 or more than 1 standard deviation per the practice in the art. Alternatively, “about” or “comprising essentially of” may mean a range of up to 10% (i.e., ±10%). For example, about 3 mg may include any number between 2.7 mg and 3.3 mg (for 10%). With respect to biological systems or processes, the terms may mean up to an order of magnitude or up to 5-fold of a value. When certain values or compositions are provided in the application and claims, unless otherwise stated, the meaning of “about” or “comprising essentially of” include an acceptable error range for that value or composition. Any concentration range, percentage range, ratio range, or integer range includes the value of any integer within the recited range and, when appropriate, fractions thereof (such as one-tenth and one-hundredth of an integer), unless otherwise indicated. As an example, “about” or “approximately” may mean within one or more than one standard deviation per the practice in the art. “About” or “approximately” may mean a range of up to 10% (i.e., ±10%). Thus, “about” may be understood to be within 10%, 9%, 8%, 7%, 6%, 5%, 4%, 3%, 2%, 1%, 0.5%, 0.1%, 0.05%, 0.01%, or 0.001% greater or less than the stated value. For example, about 5 mg may include any amount between 4.5 mg and 5.5 mg. Furthermore, particularly with respect to biological systems or processes, the terms may mean up to an order of magnitude or up to 5-fold of a value. When particular values or compositions are provided in the instant disclosure, unless otherwise stated, the meaning of “about” or “approximately” should be assumed to be within an acceptable error range for that particular value or composition.

As used herein, the term the terms “a” and “an” are used per standard convention and mean one or more, unless context dictates otherwise.

As used herein, the term “and/or” is to be understood as specific disclosure of each of the two specified features or components with or without the other. Thus, the term “and/or” as used in a phrase such as “A and/or B” herein is intended to include “A and B,” “A or B,” “A” (alone), and “B” (alone). Likewise, the term “and/or,” as used in a phrase such as “A, B, and/or C” is intended to encompass each of the following aspects: A, B, and C; A, B, or C; A or C; A or B; B or C; A and C; A and B; B and C; A (alone); B (alone); and C (alone).

The terms “e.g.,” and “i.e.,” are used merely by way of example, without limitation intended, and not be construed as referring only those items explicitly enumerated in the specification.

As used herein, the terms “or more”, “at least”, “more than”, and the like, e.g., “at least one” include but not be limited to at least 1, 2, 3, 4, 5, 6, 7, 8, 9, 10, 11, 12, 13, 14, 15, 16, 17, 18, 19 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, 101, 102, 103, 104, 105, 106, 107, 108, 109, 110, 111, 112, 113, 114, 115, 116, 117, 118, 119, 120, 121, 122, 123, 124, 125, 126, 127, 128, 129, 130, 131, 132, 133, 134, 135, 136, 137, 138, 139, 140, 141, 142, 143, 144, 145, 146, 147, 148, 149 or 150, 200, 300, 400, 500, 600, 700, 800, 900, 1000, 2000, 3000, 4000, 5000 or more than the stated value. Also included is any greater number or fraction in between. The term “no more than” includes each value less than the stated value. For example, “no more than xyx” includes 100, 99, 98, 97, 96, 95, 94, 93, 92, 91, 90, 89, 88, 87, 86, 85, 84, 83, 82, 81, 80, 79, 78, 77, 76, 75, 74, 73, 72, 71, 70, 69, 68, 67, 66, 65, 64, 63, 62, 61, 60, 59, 58, 57, 56, 55, 54, 53, 52, 51, 50, 49, 48, 47, 46, 45, 44, 43, 42, 41, 40, 39, 38, 37, 36, 35, 34, 33, 32, 31, 30, 29, 28, 27, 26, 25, 24, 23, 22, 21, 20, 19, 18, 17, 16, 15, 14, 13, 12, 11, 10, 9, 8, 7, 6, 5, 4, 3, 2, 1, and 0 xyz. Also included is any lesser number or fraction in between.

The terms “plurality”, “at least two”, “two or more”, “at least second”, and the like include but not limited to at least 2, 3, 4, 5, 6, 7, 8, 9, 10, 11, 12, 13, 14, 15, 16, 17, 18, 19 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, 101, 102, 103, 104, 105, 106, 107, 108, 109, 110, 111, 112, 113, 114, 115, 116, 117, 118, 119, 120, 121, 122, 123, 124, 125, 126, 127, 128, 129, 130, 131, 132, 133, 134, 135, 136, 137, 138, 139, 140, 141, 142, 143, 144, 145, 146, 147, 148, 149 or 150, 200, 300, 400, 500, 600, 700, 800, 900, 1000, 2000, 3000, 4000, 5000 or more. Also included is any greater number or fraction in between.

Throughout the specification the word “comprising,” or variations such as “comprises” or “comprising,” is understood to imply the inclusion of a stated element, integer or step, or group of elements, integers or steps, but not the exclusion of any other element, integer or step, or group of elements, integers or steps. It is understood that wherever aspects are described herein with the language “comprising,” otherwise analogous aspects described in terms of “consisting of” and/or “consisting essentially of” are also provided. The term “consisting of” excludes any element, step, or ingredient not specified in the claim. In re Gray, 53 F.2d 520, 11 USPQ 255 (CCPA 1931); Ex parte Davis, 80 USPQ 448, 450 (Bd. App. 1948) (“consisting of” defined as “closing the claim to the inclusion of materials other than those recited except for impurities ordinarily associated therewith”). The term “consisting essentially of” limits the scope of a claim to the specified materials or steps “and those that do not materially affect the basic and novel characteristic(s)” of the claimed invention.

As described herein, any concentration range, percentage range, ratio range or integer range is to be understood to be inclusive of the value of any integer within the recited range and, when appropriate, fractions thereof (such as one-tenth and one-hundredth of an integer), unless otherwise indicated.

The terms “administration,” “administering” or the like refer to physical introduction of an agent to a subject, using any of the various methods and delivery systems known to those skilled in the art. Exemplary routes of administration for the immune cells prepared by the methods disclosed herein include intravenous (i.v. or IV), intramuscular, subcutaneous, intraperitoneal, spinal or other parenteral routes of administration, for example by injection or infusion. Parenteral route of administration refer to modes of administration other than enteral and topical administration, usually by injection, and includes, without limitation, intravenous, intramuscular, intraarterial, intrathecal, intralymphatic, intralesional, intracapsular, intraorbital, intracardiac, intradermal, intraperitoneal, transtracheal, subcutaneous, subcuticular, intraarticular, subcapsular, subarachnoid, intraspinal, epidural and intrasternal injection and infusion, as well as in vivo electroporation. In one embodiment, the immune cells (e.g., T cells) prepared by the present methods are administered via injection or infusion. Non-parenteral routes include a topical, epidermal, or mucosal route of administration, for example, intranasally, vaginally, rectally, sublingually, or topically. Administering may also be once, twice, or a plurality of times over one or more extended periods. Where one or more therapeutic agents (e.g., cells) are administered, the administration may be done concomitantly or sequentially. Sequential administration comprises administration of one agent only after administration of the other agent or agents has been completed.

As used herein, the term “antibody” (Ab) includes, without limitation, a glycoprotein immunoglobulin which binds specifically to an antigen. In general, and antibody can comprise at least two heavy (H) chains and two light (L) chains interconnected by disulfide bonds, or an antigen-binding molecule thereof. Each H chain comprises a heavy chain variable region (abbreviated herein as VH) and a heavy chain constant region. The heavy chain constant region comprises three constant domains, CH1, CH2 and CH3. Each light chain comprises a light chain variable region (abbreviated herein as VL) and a light chain constant region. The light chain constant region comprises one constant domain, CL. The VH and VL regions can be further subdivided into regions of hypervariability, termed complementarity determining regions (CDRs), interspersed with regions that are more conserved, termed framework regions (FR).

Each VH and VL comprises three CDRs and four FRs, arranged from amino-terminus to carboxy-terminus in the following order: FR1, CDR1, FR2, CDR2, FR3, CDR3, and FR4. The variable regions of the heavy and light chains contain a binding domain that interacts with an antigen. The constant regions of the Abs may mediate the binding of the immunoglobulin to host tissues or factors, including various cells of the immune system (e.g., effector cells) and the first component (C1q) of the classical complement system. In general, human antibodies are approximately 150 kD tetrameric agents composed of two identical heavy (H) chain polypeptides (about 50 kD each) and two identical light (L) chain polypeptides (about 25 kD each) that associate with each other into what is commonly referred to as a “Y-shaped” structure. The heavy and light chains are linked or connected to one another by a single disulfide bond; two other disulfide bonds connect the heavy chain hinge regions to one another, so that the dimers are connected to one another and the tetramer is formed. Naturally-produced antibodies are also glycosylated, e.g., on the CH2 domain.

An “antigen binding molecule,” “antibody fragment” or the like refer to any portion of an antibody less than the whole. An antigen binding molecule may include the antigenic complementarity determining regions (CDRs) or an analog known in the art. Examples of antibody fragments include, but are not limited to, Fab, Fab′, F(ab′)2, and Fv fragments, dAb, linear antibodies, scFv antibodies, and multispecific antibodies formed from antigen binding molecules. In one aspect, the CD19 CAR construct comprises an anti-CD 19 single-chain FV. A “Single-chain Fv” or “scFv” antibody binding fragment comprises the variably heavy (V_{H}) and variable light (V_{L}) domains of an antibody, where these domains are present in a single polypeptide chain. Generally, the Fv polypeptide further comprises a polypeptide linker between the V_{H} and V_{L} domains, which enables the scFv to form the desired structure for antigen binding. All antibody-related terms used herein take the customary meaning in the art and are well understood by one of ordinary skill in the art.

An “antigen” refers to any molecule that provokes an immune response or is capable of being bound by an antibody or an antigen binding molecule. The immune response may involve either antibody production, or the activation of specific immunologically competent cells, or both. A person of skill in the art would readily understand that any macromolecule, including virtually all proteins or peptides, may serve as an antigen. An antigen may be endogenously expressed, i.e., expressed by genomic DNA, or may be recombinantly expressed. An antigen may be specific to a certain tissue, such as a cancer cell, or it may be broadly expressed. In addition, fragments of larger molecules may act as antigens. In some embodiments, antigens are tumor antigens.

As used herein, the term “apheresis material” means the resulting material after it has been removed from the blood of a patient. The terms “apheresis material” and “apheresis” may be used interchangeably. Examples of an apheresis material may include platelets, white blood cells, or any constituent of blood. In more specific examples, T cells may be collected from patients for later processing (e.g., T cell modification). An apheresis material may be temperature sensitive. In various embodiments, an apheresis material may comprise leukocytes. In various embodiments, an apheresis material may be suitable for undergoing the methods described herein. In various embodiments, the one or more standards and/or marker sets may be used to characterize a leukocyte population.

As used herein, the term “allogeneic” refers to any material derived from one individual which is then introduced to another individual of the same species, e.g., allogeneic T cell transplantation.

The term “autologous” refers to any material derived from the same individual to which it is later to be re-introduced. For example, the engineered autologous cell therapy method described herein involves a collection of lymphocytes from an individual (such as a donor or a patient), which are then engineered to express a CAR construct and then administered back to the same individual.

As used herein, the terms “Chimeric antigen receptor” and “CAR” may refer to a molecule engineered to comprise a binding motif and a means of activating immune cells (for example T cells such as naive T cells, central memory T cells, effector memory T cells or combination thereof) upon antigen binding. CARs are also known as artificial T cell receptors, chimeric T cell receptors or chimeric immunoreceptors. In some embodiments, a CAR comprises a binding motif, an extracellular domain, a transmembrane domain, one or more co-stimulatory domains, and an intracellular signaling domain. A T cell that has been genetically engineered to express a chimeric antigen receptor may be referred to as a CAR T cell. “Extracellular domain” (or “ECD”) refers to a portion of a polypeptide that, when the polypeptide is present in a cell membrane, is understood to reside outside of the cell membrane, in the extracellular space.

As used herein, the term “cancer” may refer to a broad group of various diseases characterized by the uncontrolled growth of abnormal cells in the body. Unregulated cell division and growth results in the formation of malignant tumors that invade neighboring tissues and may also metastasize to distant parts of the body through the lymphatic system or bloodstream. A “cancer” or “cancer tissue” may include a tumor at various stages. In various embodiments, the cancer or tumor is stage 0, such that, e.g., the cancer or tumor is very early in development and has not metastasized. In various embodiments, the cancer or tumor is stage I, such that, e.g., the cancer or tumor is relatively small in size, has not spread into nearby tissue, and has not metastasized. In various embodiments, the cancer or tumor is stage II or stage III, such that, e.g., the cancer or tumor is larger than in stage 0 or stage I, and it has grown into neighboring tissues but it has not metastasized, except potentially to the lymph nodes. In various embodiments, the cancer or tumor is stage IV, such that, e.g., the cancer or tumor has metastasized. Stage IV may also be referred to as advanced or metastatic cancer.

In various embodiments, the cancer may be selected from a tumor derived from acute lymphoblastic leukemia (ALL), acute myeloid leukemia (AML), adenoid cystic carcinoma, adrenocortical, carcinoma, AIDS-related cancers, anal cancer, appendix cancer, astrocytomas, atypical teratoid/rhabdoid tumor, central nervous system, B-cell leukemia, lymphoma or other B cell malignancies, basal cell carcinoma, bile duct cancer, bladder cancer, bone cancer, osteosarcoma and malignant fibrous histiocytoma, brain stem glioma, brain tumors, breast cancer, bronchial tumors, burkitt lymphoma, carcinoid tumors, central nervous system cancers, cervical cancer, chordoma, chronic lymphocytic leukemia (CLL), chronic myelogenous leukemia (CML), chronic myeloproliferative disorders, colon cancer, colorectal cancer, craniopharyngioma, cutaneous t-cell lymphoma, embryonal tumors, central nervous system, endometrial cancer, ependymoblastoma, ependymoma, esophageal cancer, esthesioneuroblastoma, ewing sarcoma family of tumors extracranial germ cell tumor, extragonadal germ cell tumor extrahepatic bile duct cancer, eye cancer fibrous histiocytoma of bone, malignant, and osteosarcoma, gallbladder cancer, gastric (stomach) cancer, gastrointestinal carcinoid tumor, gastrointestinal stromal tumors (GIST), soft tissue sarcoma, germ cell tumor, gestational trophoblastic tumor, glioma, hairy cell leukemia, head and neck cancer, heart cancer, hepatocellular (liver) cancer, histiocytosis, hodgkin lymphoma, hypopharyngeal cancer, intraocular melanoma, islet cell tumors (endocrine pancreas), kaposi sarcoma, kidney cancer, langerhans cell histiocytosis, laryngeal cancer, leukemia, lip and oral cavity cancer, liver cancer (primary), lobular carcinoma in situ (LCIS), lung cancer, lymphoma, macroglobulinemia, male breast cancer, malignant fibrous histiocytoma of bone and osteosarcoma, medulloblastoma, medulloepithelioma, melanoma, merkel cell carcinoma, mesothelioma, metastatic squamous neck cancer with occult primary midline tract carcinoma involving NUT gene, mouth cancer, multiple endocrine neoplasia syndromes, multiple myeloma/plasma cell neoplasm, mycosis fungoides, myelodysplastic syndromes, myelodysplastic/myeloproliferative neoplasms, myelogenous leukemia, chronic (CML), Myeloid leukemia, acute (AML), myeloma, multiple, myeloproliferative disorders, nasal cavity and paranasal sinus cancer, nasopharyngeal cancer, neuroblastoma, non-hodgkin lymphoma, non-small cell lung cancer, oral cancer, oral cavity cancer, oropharyngeal cancer, osteosarcoma and malignant fibrous histiocytoma of bone, ovarian cancer, pancreatic cancer, papillomatosis, paraganglioma, paranasal sinus and nasal cavity cancer, parathyroid cancer, penile cancer, pharyngeal cancer, pheochromocytoma, pineal parenchymal tumors of intermediate differentiation, pineoblastoma and supratentorial primitive neuroectodermal tumors, pituitary tumor, plasma cell neoplasm/multiple myeloma, pleuropulmonary blastoma, pregnancy and breast cancer, primary central nervous system (CNS) lymphoma, prostate cancer, rectal cancer, renal cell (kidney) cancer, renal pelvis and ureter, transitional cell cancer, retinoblastoma, rhabdomyosarcoma, salivary gland cancer, sarcoma, sézary syndrome, small cell lung cancer, small intestine cancer, soft tissue sarcoma, squamous cell carcinoma, squamous neck cancer, stomach (gastric) cancer, supratentorial primitive neuroectodermal tumors, t-cell lymphoma, cutaneous, testicular cancer, throat cancer, thymoma and thymic carcinoma, thyroid cancer, transitional cell cancer of the renal pelvis and ureter, trophoblastic tumor, ureter and renal pelvis cancer, urethral cancer, uterine cancer, uterine sarcoma, vaginal cancer, vulvar cancer, Waldenström macroglobulinemia, Wilms Tumor.

In various embodiment, the methods described herein may be used to treat a tumor, wherein the tumor is a lymphoma or a leukemia. Lymphoma and leukemia are cancers of the blood that specifically affect lymphocytes. All leukocytes in the blood originate from a single type of multipotent hematopoietic stem cell found in the bone marrow. This stem cell produces both myeloid progenitor cells and lymphoid progenitor cell, which then give rise to the various types of leukocytes found in the body. Leukocytes arising from the myeloid progenitor cells include T lymphocytes (T cells), B lymphocytes (B cells), natural killer cells, and plasma cells. Leukocytes arising from the lymphoid progenitor cells include megakaryocytes, mast cells, basophils, neutrophils, eosinophils, monocytes, and macrophages. Lymphomas and leukemias may affect one or more of these cell types in a patient.

In general, lymphomas may be divided into at least two sub-groups: Hodgkin lymphoma and non-Hodgkin lymphoma. Non-Hodgkin Lymphoma (NHL) is a heterogeneous group of cancers originating in B lymphocytes, T lymphocytes or natural killer cells. In the United States, B cell lymphomas represent 80-85% of cases reported. In 2013 approximately 69,740 new cases of NHL and over 19,000 deaths related to the disease were estimated to occur. Non-Hodgkin lymphoma is the most prevalent hematological malignancy and is the seventh leading site of new cancers among men and women and account for 4% of all new cancer cases and 3% of deaths related to cancer.

In various embodiments, the methods described herein may be used to treat a lymphoma or a leukemia. In various embodiments, the lymphoma or leukemia may be a B cell malignancy. Examples of B cell malignancies include, but are not limited to, Non-Hodgkin's Lymphomas (NHL), Small lymphocytic lymphoma (SLL/CLL), Mantle cell lymphoma (MCL), FL, Marginal zone lymphoma (MZL), Extranodal (MALT lymphoma), Nodal (Monocytoid B-cell lymphoma), Splenic, Diffuse large cell lymphoma, B cell chronic lymphocytic leukemia/lymphoma, Burkitt's lymphoma, and Lymphoblastic lymphoma. In some aspects, the lymphoma or leukemia is selected from B-cell chronic lymphocytic leukemia/small cell lymphoma. B-cell prolymphocytic leukemia, lymphoplasmacytic lymphoma (e.g., Waldenström macroglobulinemia), splenic marginal zone lymphoma, hairy cell leukemia, plasma cell neoplasms (e.g., plasma cell myeloma (i.e., multiple myeloma), or plasmacytoma), extranodal marginal zone B cell lymphoma (e.g., MALT lymphoma), nodal marginal zone B cell lymphoma, follicular lymphoma (FL), transformed follicular lymphoma (TFL), primary cutaneous follicle center lymphoma, mantle cell lymphoma, diffuse large B cell lymphoma (DLBCL), Epstein-Barr virus-positive DLBCL, lymphomatoid granulomatosis, primary mediastinal (thymic) large B-cell lymphoma (PMBCL), Intravascular large B-cell lymphoma, ALK+ large B-cell lymphoma, plasmablastic lymphoma, primary effusion lymphoma, large B-cell lymphoma arising in HHV8-associated multicentric Castleman's disease, Burkitt lymphoma/leukemia, T-cell prolymphocytic leukemia, T-cell large granular lymphocyte leukemia, aggressive NK cell leukemia, adult T-cell leukemia/lymphoma, extranodal NK/T-cell lymphoma, enteropathy-associated T-cell lymphoma, Hepatosplenic T-cell lymphoma, blastic NK cell lymphoma, Mycosis fungoides/Sezary syndrome, Primary cutaneous anaplastic large cell lymphoma. Lymphomatoid papulosis, Peripheral T-cell lymphoma, Angioimmunoblastic T cell lymphoma, Anaplastic large cell lymphoma, B-lymphoblastic leukemia/lymphoma, B-lymphoblastic leukemia/lymphoma with recurrent genetic abnormalities, T-lymphoblastic leukemia/lymphoma, and Hodgkin lymphoma. In some aspect, the cancer is refractory to one or more prior treatments, and/or the cancer has relapsed after one or more prior treatments.

In various embodiments, the cancer may be selected from follicular lymphoma, transformed follicular lymphoma, diffuse large B cell lymphoma, and primary mediastinal (thymic) large B-cell lymphoma. In various embodiment, the cancer may be diffuse large B cell lymphoma. In various embodiment, the cancer may be refractory to or the cancer has relapsed following one or more of chemotherapy, radiotherapy, immunotherapy (including a T cell therapy and/or treatment with an antibody or antibody-drug conjugate), an autologous stem cell transplant, or any combination thereof. In various embodiments, the cancer may be refractory diffuse large B cell lymphoma or mantle cell lymphoma.

As used herein, the term “lymphocyte” may include natural killer (NK) cells, T cells, NK-T cells, monocytes, early progenitor B cells, stem cells, and B cells. NK cells are a type of cytotoxic (cell toxic) lymphocyte that represent a major component of the inherent immune system. NK cells reject tumors and cells infected by viruses through the process of apoptosis or programmed cell death. NK cells were termed “natural killers” because they do not require activation to kill cells. T cells play a major role in cell-mediated immunity (no antibody involvement). T-cell receptors (TCR) may differentiate themselves from other lymphocyte types. The thymus, a specialized organ of the immune system, is primarily responsible for the T cell's maturation.

There are several types of “immune cells,” including, without limitation, macrophages (e.g., tumor associated macrophages) neutrophils, basophils, eosinophils, granulocytes, natural killer cells (NK cells), B cells, T cells, NK-T cells, mast cells, tumor infiltrating lymphocytes (TILs), myeloid derived suppressor cells (MDSCs), and dendritic cells. The term “immune cells” also includes precursors of these immune cells. Hematopoietic stem and/or progenitor cells may be derived from bone marrow, umbilical cord blood, adult peripheral blood after cytokine mobilization, and the like, by methods known in the art. Some precursor cells are those that may differentiate into the lymphoid lineage, for example, hematopoietic stem cells or progenitor cells of the lymphoid lineage. Additional examples of immune cells that may be used for immune therapy are described in US Publication No. 20180273601, which is incorporated herein by reference in its entirety.

There are also several types of T-cells, namely: Helper T-cells (e.g., CD4+ cells, effector TEF cells), Cytotoxic T-cells (also known as TC, cytotoxic T lymphocyte, CTL, T-killer cell, cytolytic T cell, CD8+ T-cells or killer T cell), Memory T-cells ((i) stem memory T_{SCM} cells, like naive cells, are CD45RO−, CCR7+, CD45RA+, CD62L+(L-selectin), CD27+, CD28+ and IL-7Ra+, but they also express large amounts of CD95, IL-2Rβ, CXCR3, and LFA-1, and show numerous functional attributes distinctive of memory cells); (ii) central memory T_{SCM} cells express L-selectin and are CCR7+ and CD45RO⁺ and they secrete IL-2, but not IFNγ or IL-4, and (iii) effector memory T_{EM} cells, however, do not express L-selectin or CCR7 but do express CD45RO and produce effector cytokines like IFNγ and IL-4), Regulatory T-cells (Tregs, suppressor T cells, or CD4+CD25+ regulatory T cells), Natural Killer T-cells (NKT), and Gamma Delta T-cells. T cells found within tumors are referred to as “tumor infiltrating lymphocytes” (TIL). B-cells, on the other hand, play a principal role in humoral immunity (with antibody involvement). It makes antibodies and antigens and performs the role of antigen-presenting cells (APCs) and turns into memory B-cells after activation by antigen interaction. In mammals, immature B-cells are formed in the bone marrow, from where its name is derived.

A “naïve” T cell refers to a mature T cell that remains immunologically undifferentiated. Following positive and negative selection in the thymus, T cells emerge as either CD4⁺ or CD8⁺ naïve T cells. In their naïve state, T cells express L-selectin (CD62L⁺), IL-7 receptor-α (IL-7R-α), and CD132, but they do not express CD25, CD44, CD69, or CD45RO. As used herein, “immature” may also refer to a T cell which exhibits a phenotype characteristic of either a naïve T cell or an immature T cell, such as a T_{SCM} cell or a T_{SCM} cell. For example, an immature T cell may express one or more of L-selectin (CD62L⁺), IL-7Rα, CD132, CCR7, CD45RA, CD45RO, CD27, CD28, CD95, IL-2Rβ, CXCR3, and LFA-1. Naïve or immature T cells may be contrasted with terminal differentiated effector T cells, such as T_{EM} cells and T_{EFF} cells.

As used herein the terms cell “proliferation,” “proliferating” or the like refer to the ability of cells to grow in numbers through cell division. Proliferation may be measured by staining cells with carboxyfluorescein succinimidyl ester (CFSE). Cell proliferation may occur in vitro, e.g., during T cell culture, or in vivo, e.g., following administration of a immune cell therapy (e.g., T cell therapy). The cell proliferation may be measured or determined by the methods described herein or known in the field. For example, cell proliferation may be measured or determined by viable cell density (VCD) or total viable cell (TVC). VCD or TVC may be theoretical (an aliquot or sample is removed from a culture at certain timepoint to determine the cell number, then the cell number multiples with the culture volume at the beginning of the study) or actual (an aliquot or sample is removed from a culture at certain timepoint to determine the cell number, then the cell number multiples with the actual culture volume at the certain timepoint). The term “T cell activity” refers to any activity common to healthy T cells. In various embodiments, the T cell activity comprises cytokine production (such as INPγ, IL-2, and/or TNFα). In various embodiments, the T cell activity comprises production of one or more cytokine selected from interferon gamma (IFNγ or IFN-γ), tissue necrosis factor alpha (TNFα or IFNα), and both. The terms “cytolytic activity,” “cytotoxicity” or the like refer to the ability of a T cell to destroy a target cell. In various embodiments, the target cell may be a cancer cell, e.g., a tumor cell. In various embodiments, the T cell may express a chimeric antigen receptor (CAR) or a T cell receptor (TCR), and the target cell may express a target antigen.

As used herein, the terms “genetically engineered,” “gene editing,” or “engineered” may refer to a method of modifying the genome of a cell, including, but not being limited to, deleting a coding or non-coding region or a portion thereof or inserting a coding region or a portion thereof. In various embodiment, the cell that is modified is a lymphocyte, e.g., a T cell, which may either be obtained from a patient or a donor. The cell may be modified to express an exogenous construct, such as, e.g., a chimeric antigen receptor (CAR) or a T cell receptor (TCR), which may be incorporated into the cell's genome.

The terms “transduction” and “transduced” refer to the process whereby foreign DNA is introduced into a cell via viral vector (see Jones et al., “Genetics: principles and analysis,” Boston: Jones & Bartlett Publ. (1998)). In various embodiments, the vector may be a retroviral vector, a DNA vector, a RNA vector, an adenoviral vector, a baculoviral vector, an Epstein Barr viral vector, a papovaviral vector, a vaccinia viral vector, a herpes simplex viral vector, an adenovirus associated vector, a lentiviral vector, or any combination thereof.

As used herein, the terms “T cell receptor” or “TCR” may refer to antigen-recognition molecules present on the surface of T cells. During normal T cell development, each of the four TCR genes, α, β, γ, and δ, may rearrange leading to highly diverse TCR proteins.

The “antigen binding molecule” may comprise a binding molecule to a tumor antigen. The binding molecule may be an antibody or an antigen binding molecule thereof. For example, the antigen binding molecule may be selected from scFv, Fab, Fab′, Fv, F(ab′)2, and dAb, and any fragments or combinations thereof. The chimeric antigen receptor may further comprise a hinge region. The hinge region may be derived from the hinge region of IgG1, IgG2, IgG3, IgG4, IgA, IgD, IgE, IgM, CD28, or CD8 alpha. In various embodiments, the hinge region may be derived from the hinge region of IgG4. The chimeric antigen receptor may also comprise a transmembrane domain. The transmembrane domain may be a transmembrane domain of any transmembrane molecule that is a co-receptor on immune cells or a transmembrane domain of a member of the immunoglobulin superfamily. In various embodiments, the transmembrane domain is derived from a transmembrane domain of CD28, CD28T, CD8 alpha, CD4, or CD19. In various embodiments, the transmembrane domain comprises a domain derived from a CD28 transmembrane domain. In various embodiment, the transmembrane domain comprises a domain derived from a CD28T transmembrane domain.

The “antigen” may be the tumor antigen selected from 707-AP (707 alanine proline), AFP (alpha (a)-fetoprotein), ART-4 (adenocarcinoma antigen recognized by T4 cells), BAGE (B antigen; b-catenin/m, b-catenin/mutated), BCMA (B cell maturation antigen), Bcr-abl (breakpoint cluster region-Abelson), CAIX (carbonic anhydrase IX), CD19 (cluster of differentiation 19), CD20 (cluster of differentiation 20), CD22 (cluster of differentiation 22), CD30 (cluster of differentiation 30), CD33 (cluster of differentiation 33), CD44v7/8 (cluster of differentiation 44, exons 7/8), CAMEL (CTL-recognized antigen on melanoma), CAP-1 (carcinoembryonic antigen peptide-1), CASP-8 (caspase-8), CDC27m (cell-division cycle 27 mutated), CDK4/m (cycline-dependent kinase 4 mutated), CEA (carcinoembryonic antigen), CT (cancer/testis (antigen)), Cyp-B (cyclophilin B), DAM (differentiation antigen melanoma), EGFR (epidermal growth factor receptor), EGFRvII (epidermal growth factor receptor, variant III), EGP-2 (epithelial glycoprotein 2), EGP-40 (epithelial glycoprotein 40), Erbb2, 3, 4 (erythroblastic leukemia viral oncogene homolog-2, -3, 4), ELF2M (elongation factor 2 mutated), ETV6-AML1 (Ets variant gene 6/acute myeloid leukemia 1 gene ETS), FBP (folate binding protein), fAchR (Fetal acetylcholine receptor), G250 (glycoprotein 250), GAGE (G antigen), GD2 (disialoganglioside 2), GD3 (disialoganglioside 3), GnT-V (N-acetylglucosaminyltransferase V), Gp100 (glycoprotein 100 kD), HAGE (helicose antigen), HER-2/neu (human epidermal receptor-2/neurological; also known as EGFR2). HLA-A (human leukocyte antigen-A) HPV (human papilloma virus), HSP70-2M (heat shock protein 70-2 mutated), HST-2 (human signet ring tumor-2), hTERT or hTRT (human telomerase reverse transcriptase), iCE (intestinal carboxyl esterase), IL-13R-a2 (Interleukin-13 receptor subunit alpha-2), KIAA0205, KDR (kinase insert domain receptor), K-light chain, LAGE (L antigen), LDLR/FUT (low density lipid receptor/GDP-L-fucose: b-D-galactosidase 2-a-Lfucosyltransferase), LeY (Lewis-Y antibody), LiCAM (L1 cell adhesion molecule), MAGE (melanoma antigen), MAGE-A1 (Melanoma-associated antigen 1), MAGE-A3, MAGE-A6, mesothelin, Murine CMV infected cells, MART-1/Melan-A (melanoma antigen recognized by T cells-1/Melanoma antigen A), MC1R (melanocortin 1 receptor), Myosin/m (myosin mutated), MUC1 (mucin 1), MUM-1, -2, -3 (melanoma ubiquitous mutated 1, 2, 3), NA88-A (NA cDNA clone of patient M88), NKG2D (Natural killer group 2, member D) ligands, NY-BR-1 (New York breast differentiation antigen 1), NY-ESO-1 (New York esophageal squamous cell carcinoma-1), oncofetal antigen (h5T4), P15 (protein 15), p190 minor bcr-abl (protein of 190 KD ber-abl), Pml/RARa (promyelocytic leukaemia/retinoic acid receptor a), PRAME (preferentially expressed antigen of melanoma), PSA (prostate-specific antigen), PSCA (Prostate stem cell antigen), PSMA (prostate-specific membrane antigen), RAGE (renal antigen), RU1 or RU2 (renal ubiquitous 1 or 2). SAGE (sarcoma antigen). SART-1 or SART-3 (squamous antigen rejecting tumor 1 or 3), SSX1, -2, -3, 4 (synovial sarcoma X1, -2, -3, -4), TAA (tumor-associated antigen), TAG-72 (Tumor-associated glycoprotein 72), TEL/AML1 (translocation Ets-family leukemia/acute myeloid leukemia 1), TPI/m (triosephosphate isomerase mutated). TRP-1 (tyrosinase related protein 1, or gp75), TRP-2 (tyrosinase related protein 2), TRP-2/INT2 (TRP-2/intron 2), VEGF-R2 (vascular endothelial growth factor receptor 2), WT1 (Wilms' tumor gene), and any combination thereof. In various embodiments, the tumor antigen may be CD19.

In various embodiments, the T cell products of the disclosure are used in “CD19-directed genetically modified autologous T cell immunotherapy,” which refers to a suspension of chimeric antigen receptor (CAR)-positive immune cells. A non-limiting example of such immunotherapy is Clear CAR-T therapy, which uses CAR-T cells that are free of circulating tumor cells and enriched in CD4+/CD8+ T cells. Another example is axicabtagene ciloleucel (also known as Axi-Cel™, YESCARTA®). See Kochenderfer, et al., (J Immunother 2009; 32:689 702). In various embodiments, the T cell product is brexucabtagene autoleucel (formerly KTE-X19; Tecartus) Other non-limiting examples include JCAR017, JCAR015, JCAR014, Kymriah (tisagenlecleucel), Uppsala U. anti-CD19 CAR (NCT02132624), and UCART19 (Celectis). See Sadelain et al. Nature Rev. Cancer Vol. 3 (2003), Ruella et al., Curr Hematol Malig Rep., Springer, NY (2016) and Sadelain et al. Cancer Discovery (April 2013) To prepare CD19-directed genetically modified autologous T cell immunotherapy, a patient's own T cells may be harvested and genetically modified ex vivo by retroviral transduction to express a chimeric antigen receptor (CAR) comprising a murine anti-CD19 single chain variable fragment (scFv) linked to CD28 and CD3-zeta co-stimulatory domains. In various embodiments, the CAR comprises a murine anti-CD19 single chain variable fragment (scFv) linked to 4-1BB and CD3-zeta co-stimulatory domain. The anti-CD19 CAR T cells may be expanded and infused back into the patient, where they may recognize and eliminate CD19-expressing target cells.

In various embodiments, the T cells may be engineered with a T cell receptor (TCR), which may comprise a binding molecule to a tumor antigen. In various aspects, the tumor antigen is selected from the group consisting of 707-AP, AFP, ART-4, BAGE, BCMA, Bcr-abl, CAIX, CD19, CD20, CD22, CD30, CD33, CD44v7/8, CAMEL, CAP-1, CASP-8, CDC27m, CDK4/m, CEA, CT, Cyp-B, DAM, EGFR, EGFRvIII, EGP-2, EGP-40, Erbb2, 3, 4, ELF2M, ETV6-AML1, FBP, fAchR, G250, GAGE, GD2, GD3, GnT-V, Gp100, HAGE, HER-2/neu, HLA-A, HPV, HSP70-2M, HST-2, hTERT or hTRT, iCE, IL-13R-a2, KIAA0205, KDR, κ-light chain, LAGE, LDLR/FUT, LeY, L1CAM, MAGE, MAGE-A1, mesothelin, Murine CMV infected cells, MART-1/Melan-A, MC1R, Myosin/m, MUC1, MUM-1, -2, -3, NA88-A, NKG2D ligands, NY-BR-1, NY-ESO-1, oncofetal antigen, P15, p190 minor ber-abl, Pml/RARa, PRAME, PSA, PSCA, PSMA, RAGE, RUI or RU2, SAGE, SART-1 or SART-3, SSX1, -2, -3,4, TAA, TAG-72, TEL/AML1, TPI/m, TRP-1, TRP-2, TRP-2/INT2, VEGF-R2, WT1, and any combination thereof. In one aspect, the TCR comprises a binding molecule to a viral oncogene. In various embodiments, the viral oncogene is selected from human papilloma virus (HPV), Epstein-Barr virus (EBV), and human T-lymphotropic virus (HTLV). In various embodiments, the TCR comprises a binding molecule to a testicular, placental, or fetal tumor antigen. In various embodiment, the testicular, placental, or fetal tumor antigen is selected from the group consisting of NY-ESO-1, synovial sarcoma X breakpoint 2 (SSX2), melanoma antigen (MAGE), and any combination thereof. In another embodiment, the TCR comprises a binding molecule to a lineage specific antigen. In various embodiments, the lineage specific antigen is selected from the group consisting of melanoma antigen recognized by T cells 1 (MART-1), gp100, prostate specific antigen (PSA), prostate specific membrane antigen (PSMA), prostate stem cell antigen (PSCA), and any combination thereof. In various embodiments, the T cell therapy comprises administering to the patient engineered CAR T cells expressing a chimeric antigen receptor that binds to CD19 and further comprises a CD28 costimulatory domain and a CD3-zeta signaling region. In various aspects, the antigenic moieties may also include, but are not limited to, an Epstein-Barr virus (EBV) antigen (e.g., EBNA-1, EBNA-2, EBNA-3, LMP-1, LMP-2), a hepatitis A virus antigen (e.g., VP1, VP2, VP3), a hepatitis B virus antigen (e.g., HBsAg, HBcAg, HBeAg), a hepatitis C viral antigen (e.g., envelope glycoproteins E1 and E2), a herpes simplex virus type 1, type 2, or type 8 (HSV1, HSV2, or HSV8) viral antigen (e.g., glycoproteins gB, gC, gC, gE, gG, gH, gI, gJ, gK, gL, gM, UL20, UL32, US43, UL45, UL49A), a cytomegalovirus (CMV) viral antigen (e.g., glycoproteins gB, gC, gC, gE, gG, gH, gI, gJ, gK, gL, gM or other envelope proteins), a human immunodeficiency virus (HIV) viral antigen (glycoproteins gp120, gp41, or p24), an influenza viral antigen (e.g., hemagglutinin (HA) or neuraminidase (NA)), a measles or mumps viral antigen, a human papillomavirus (HPV) viral antigen (e.g., L1, L2), a parainfluenza virus viral antigen, a rubella virus viral antigen, a respiratory syncytial virus (RSV) viral antigen, or a varicella-zostser virus viral antigen. In such aspects, the cell surface receptor may be any TCR, or any CAR which recognizes any of the aforementioned viral antigens on a target virally infected cell. In other aspects, the antigenic moiety is associated with cells having an immune or inflammatory dysfunction. Such antigenic moieties may include, but are not limited to, myelin basic protein (MBP) myelin proteolipid protein (PLP), myelin oligodendrocyte glycoprotein (MOG), carcinoembryonic antigen (CEA), pro-insulin, glutamine decarboxylase (GAD65, GAD67), heat shock proteins (HSPs), or any other tissue specific antigen that is involved in or associated with a pathogenic autoimmune process.

As used herein, the terms “immunotherapy” “immune therapy” or the like may refer to the treatment of a subject afflicted with, or at risk of contracting or suffering a recurrence of, a disease by a method comprising inducing, enhancing, suppressing or otherwise modifying an immune response. Examples of immunotherapy include, but are not limited to, T cell and NK cell therapies. T cell therapy may include adoptive T cell therapy, tumor-infiltrating lymphocyte (TIL) immunotherapy, autologous cell therapy, engineered autologous cell therapy and allogeneic T cell transplantation. One of skill in the art would recognize that the methods of preparing immune cells disclosed herein would enhance the effectiveness of any cancer or transplanted T cell therapy. Examples of T cell therapies are described in U.S. Patent Publication Nos. 2014/0154228 and 2002/0006409; U.S. Pat. Nos. 7,741,465; 6,319,494; and 5,728,388; and PCT Publication No. WO 2008/081035, which are incorporated by reference in their entirety.

The one or more immune cells described herein may be obtained from any source, including, for example, a human donor. The donor may be a subject in need of an anti-cancer treatment, e.g., treatment with one immune cells generated by the methods described herein (i.e., an autologous donor), or may be an individual that donates a lymphocyte sample that, upon generation of the population of cells generated by the methods described herein, will be used to treat a different individual or cancer patient (i.e., an allogeneic donor). immune cells may be differentiated in vitro from a hematopoietic stem cell population, or immune cells may be obtained from a donor. The population of immune cells may be obtained from the donor by any suitable method used in the art. For example, the population of lymphocytes may be obtained by any suitable extracorporeal method, venipuncture, or other blood collection method by which a sample of blood with or without lymphocytes is obtained. The population of lymphocytes may be obtained by apheresis. The one or more immune cells may be collected from any tissue that comprises one or more immune cells, including, but not limited to, a tumor. A tumor or a portion thereof may be collected from a subject, and one or more immune cells are isolated from the tumor tissue. Any T cell may be used in the methods disclosed herein, including any immune cells suitable for a T cell therapy. For example, the one or more cells useful for the application may be selected from the group consisting of tumor infiltrating lymphocytes (TIL), cytotoxic T cells, CAR T cells, engineered TCR T cells, natural killer T cells, Dendritic cells, and peripheral blood lymphocytes. T cells may be obtained from, e.g., peripheral blood mononuclear cells, bone marrow, lymph node tissue, cord blood, thymus tissue, tissue from a site of infection, ascites, pleural effusion, spleen tissue, and tumors. In addition, the T cells may be derived from one or more T cell lines available in the art. T cells may also be obtained from a unit of blood collected from a subject using any number of techniques known to the skilled artisan, such as FICOLL™ separation and/or apheresis. T cells may also be obtained from an artificial thymic organoid (ATO) cell culture system, which replicates the human thymic environment to support efficient ex vivo differentiation of T-cells from primary and reprogrammed pluripotent stem cells. Additional methods of isolating T cells for a T cell therapy are disclosed in U.S. Patent Publication No. 2013/0287748, in PCT Publication Nos. WO2015/120096 and WO2017/070395, all of which are herein incorporated by reference in their totality for the purposes of describing these methods and in their entirety. In various embodiments, T cells may be tumor infiltrating leukocytes. In various embodiments, the one or more T cells may express CD8, e.g., are CD8⁺ T cells. In various embodiments, the one or more T cells may express CD4, e.g., are CD4⁺ T cells. Additional methods of isolating T cells for a T cell therapy are disclosed in U.S. Patent Publication No. 2013/0287748, in PCT Publication Nos. WO2015/120096 and WO2017/070395, all of which are herein incorporated by reference in their totality for the purposes of describing these methods and in their entirety. In various aspects, the cells of the present application may be obtained through T cells obtained from a subject. In various aspects, the T cells may be obtained from, e.g., peripheral blood mononuclear cells (PBMC), bone marrow, lymph node tissue, cord blood, thymus tissue, tissue from a site of infection, ascites, pleural effusion, spleen tissue, and tumors. In addition, the T cells may be derived from one or more T cell lines available in the art. T cells may also be obtained from a unit of blood collected from a subject using any number of techniques known to the skilled artisan, such as FICOLL™ separation and/or apheresis. In various aspects, the cells collected by apheresis are washed to remove the plasma fraction and placed in an appropriate buffer or media for subsequent processing. In various aspects, the cells may be washed with any solution (e.g., a solution with neutralized PH value or PBS) or culture medium. As will be appreciated by the skilled artisan, a washing step may be used, such as by using a semiautomated flow through centrifuge, e.g., the Cobe™ 2991 cell processor, the Baxter CytoMate™, or the like. In various aspects, the washed cells may be resuspended in one or more biocompatible buffers, or other saline solution with or without buffer. In various aspects, the undesired components of the apheresis sample may be removed. Additional methods of isolating T cells for a T cell therapy are disclosed in U.S. Patent Pub. No. 2013/0287748, which is hereby incorporated by reference in their entirety.

In various embodiments, T cells may be isolated from PBMCs by lysing the red blood cells and depleting the monocytes, e.g., by using centrifugation through a PERCOLL™ gradient.

In various embodiments, a specific subpopulation of T cells, such as CD4+, CD8+, CD28+, CD45RA+, and CD45RO+ T cells may be further isolated by positive or negative selection techniques known in the art. For example, enrichment of a T cell population by negative selection may be accomplished with a combination of antibodies directed to surface markers unique to the negatively selected cells. In various embodiments, cell sorting and/or selection via negative magnetic immunoadherence or flow cytometry that uses a cocktail of monoclonal antibodies directed to cell surface markers present on the cells negatively selected may be used. For example, to enrich for CD4+ cells by negative selection, a monoclonal antibody cocktail typically includes antibodies to CD8, CD11b, CD14, CD16, CD20, and HLA-DR. In some embodiments, flow cytometry and cell sorting are used to isolate cell populations of interest for use in the present disclosure.

In various embodiments, CD3+ T cells may be isolated from PBMCs using Dynabeads coated with anti-CD3 antibody. CD8+ and CD4+ T cells are further separately isolated by positive selection using CD8 microbeads (e.g., Miltenyi Biotec) and/or CD4 microbeads (e.g., Miltenyi Biotec).

PBMCs may be used directly for genetic modification with the immune cells (such as CARs). After isolating the PBMCs, T lymphocytes may be further isolated, and both cytotoxic and helper T lymphocytes are sorted into naïve, memory, and effector T cell subpopulations either before or after genetic modification and/or expansion. In various embodiments, CD8+ cells may be further sorted into naïve, central memory, and effector cells by identifying cell surface antigens that are associated with each of these types of CD8+ cells. In various embodiments, the expression of phenotypic markers of central memory T cells includes CCR7, CD3, CD28, CD45RO, CD62L, and CD127 and are negative for granzyme B. In various embodiments, central memory T cells are CD8+, CD45RO+, and CD62L+ T cells. In various embodiments, effector T cells may be negative for CCR7, CD28, CD62L, and CD127 and positive for granzyme B and perforin. In various embodiments, CD4+ T cells may be further sorted into subpopulations. For example, CD4+T helper cells may be sorted into naïve, central memory, and effector cells by identifying cell populations that have cell surface antigens.

The methods described herein further comprise enriching or preparing a population of immune cells obtained from a donor, between harvesting from the donor and exposing one or more cells obtained from a donor subject. Enrichment of a population of immune cells, e.g., the one or more T cells, may be accomplished by any suitable separation method including, but not limited to, the use of a separation medium (e.g., FICOLL-PAQUE™, ROSETTESEP™ HLA Total Lymphocyte enrichment cocktail, Lymphocyte Separation Medium (LSA) (MP Biomedical Cat. No. 0850494X), or the like), cell size, shape or density separation by filtration or elutriation, immunomagnetic separation (e.g., magnetic-activated cell sorting system, MACS), fluorescent separation (e.g., fluorescence activated cell sorting system, FACS), or bead-based column separation.

In various embodiments, the T cell preparations described herewith may be used for engineered Autologous Cell Therapy. The term “engineered Autologous Cell Therapy,” which may be abbreviated as “eACT™,” also known as adoptive cell transfer, is a process by which a patient's own T cells are collected and subsequently genetically altered to recognize and target one or more antigens expressed on the cell surface of one or more specific tumor cells or malignancies. T cells may be engineered to express, for example, chimeric antigen receptors (CAR) or T cell receptor (TCR). CAR positive (+) T cells are engineered to express an extracellular single chain variable fragment (scFv) with specificity for certain tumor antigen linked to an intracellular signaling part comprising a costimulatory domain and an activating domain.

In various embodiments, the donor T cells for use in the T cell therapy may be obtained from the patient (e.g., for an autologous T cell therapy). In various embodiments, the donor T cells for use in the T cell therapy may be obtained from a subject that is not the patient. The T cells may be administered at a therapeutically effective amount. For example, a therapeutically effective amount of the T cells may be at least about 10⁴ cells, at least about 10⁵ cells, at least about 10⁶ cells, at least about 10⁷ cells, at least about 10⁸ cells, at least about 10⁹, or at least about 10¹⁰. In various embodiments, the therapeutically effective amount of the T cells is about 10⁴ cells, about 10 cells, about 10⁶ cells, about 10⁷ cells, or about 10⁸ cells. In various embodiments, the therapeutically effective amount of the CAR T cells may be about 2×10⁶ cells/kg, about 3×10⁶ cells/kg, about 4×10⁶ cells/kg, about 5×10⁶ cells/kg, about 6×10⁶ cells/kg, about 7×10⁶ cells/kg, about 8×10⁶ cells/kg, about 9×10⁶ cells/kg, about 1×10⁷ cells/kg, about 2×10⁷ cells/kg, about 3×10⁷ cells/kg, about 4×10⁷ cells/kg, about 5×10⁷ cells/kg, about 6×10⁷ cells/kg, about 7×10⁷ cells/kg, about 8×10⁷ cells/kg, or about 9×10⁷ cells/kg. In some embodiments, the therapeutically effective amount of the CAR-positive viable T cells is between about 1×10⁶ and about 2×10⁶ CAR-positive viable T cells per kg body weight up to a maximum dose of about 1×10⁸ CAR-positive viable T cells. In various embodiments, the therapeutically effective amount of the CAR-positive viable T cells may be between about 0.4×10⁸ and about 2×10⁸ CAR-positive viable T cells. In various embodiments, the therapeutically effective amount of the CAR-positive viable T cells may be about 0.4×10⁸, about 0.5×10⁸, about 0.6×10⁸, about 0.7×10⁸, about 0.8×10⁸, about 0.9×10⁸, about 1.0×10⁸, about 1.1×10⁸, about 1.2×10⁸, about 1.3×10⁸, about 1.4×10⁸, about 1.5×10⁸, about 1.6×10⁸, about 1.7×10⁸, about 1.8×10⁸, about 1.9×10¹, or about 2.0×10⁸ CAR-positive viable T cells.

As used herein, the term “patient” means any human who is being treated for an abnormal physiological condition, such as cancer or has been formally diagnosed with a disorder, those without formally recognized disorders, those receiving medical attention, those at risk of developing the disorders, etc. The terms “subject” and “patient” may be used interchangeably herein and include both human and non-human animal subjects.

The term “reference” describes a standard or control relative to which a comparison is performed. For example, in various embodiments, an agent, animal, individual, population, sample, sequence, or value of interest is compared with a reference or control that is an agent, animal, individual, population, sample, sequence, or value. In various embodiments, a reference or control is tested, measured, and/or determined substantially simultaneously with the testing, measuring, or determination of interest. In various embodiments, a reference or control is a historical reference or control, optionally embodied in a tangible medium. Generally, a reference or control may be determined or characterized under comparable conditions or circumstances to those under assessment. When sufficient similarities are present to justify reliance on and/or comparison to a selected reference or control.

“Treatment” or “treating” of a subject refers to any type of intervention or process performed on, or the administration of an active agent to, the subject with the objective of reversing, alleviating, ameliorating, inhibiting, slowing down or preventing the onset, progression, development, severity or recurrence of a symptom, complication or condition, or biochemical indicia associated with a disease. In one embodiment, “treatment” or “treating” includes a partial remission. In another embodiment, “treatment” or “treating” includes a complete remission. In some embodiments, treatment may be of a subject who does not exhibit signs of the relevant disease, disorder and/or condition and/or of a subject who exhibits only early signs of the disease, disorder, and/or condition. In various embodiments, such treatment may be of a subject who exhibits one or more established signs of the relevant disease, disorder and/or condition. In some embodiments, treatment may be of a subject who has been diagnosed as suffering from the relevant disease, disorder, and/or condition. In some embodiments, treatment may be of a subject known to have one or more susceptibility factors that are statistically correlated with increased risk of development of the relevant disease, disorder, and/or condition.

The term “vector” refers to a recipient nucleic acid molecule modified to comprise or incorporate a provided nucleic acid sequence. One type of vector is a “plasmid,” which refers to a circular double stranded DNA molecule into which additional DNA may be ligated. Another type of vector is a viral vector, wherein additional DNA segments may be ligated into the viral genome. Certain vectors are capable of autonomous replication in a host cell into which they are introduced (e.g., bacterial vectors having a bacterial origin of replication and episomal mammalian vectors). Other vectors (e.g., non-episomal mammalian vectors) may be integrated into the genome of a host cell upon introduction into the host cell, and thereby are replicated along with the host genome. Moreover, certain vectors comprise sequences that direct expression of inserted genes to which they are operatively linked. Such vectors may be referred to herein as “expression vectors.” Standard techniques may be used for engineering of vectors, e.g., as found in Sambrook et al., Molecular Cloning: A Laboratory Manual (2d ed., Cold Spring Harbor Laboratory Press, Cold Spring Harbor, N.Y. (1989)), which is incorporated herein by reference.

TABLE 1
Term Definitions

| Term | Definition |
|---|---|
| ALL | Acute Lymphocytic Leukemia |
| NHL | Non-Hodgkin's Lymphoma |
| Allophycocyanin | An intensely bright phycobiliprotein isolated |
| (APC) | from red algae. It has excitation/emission |
| | spectrum peak wavelengths of 594-633/660 nm. |
| Brilliant Violet | A polymer-based dye with excitation/emission |
| 421 (BV421) | spectrum peak wavelengths of 407/421 nm. |
| CV | Coefficient of variation |
| STDEV | Standard Deviation |
| FC | Flow cytometry |
| Fluorescein | A bright green fluorophore with excitation/ |
| isothiocyanate | emission spectrum peak wavelengths of |
| (FITC) | 494/520 nm. |
| LLOQ | Lower limit of quantitation |
| MFI | Median fluorescent intensity |
| Near-IR Dead Cell | Viability dye used to determine the viability |
| Stain Kit | of cells. It has excitation/emission spectrum |
| | peak wavelengths of 633/750 nm. |
| NK/NKT cells | Natural killer cells or natural killer T cells |
| PE-Cy7 | A tandem conjugate that combines PE and a |
| | cyanine dye Cy7. It has excitation/emission |
| | spectrum peak wavelengths of 496/785 nm. |
| PerCP-Cy5.5 | A tandem conjugate that combines a protein |
| | complex called PerCP and a cyanine dye Cy5.5. |
| | It has excitation/emission spectrum peak |
| | wavelengths of 482/676 nm. |
| PBMC | Peripheral blood mono-nuclear cells, any |
| | peripheral blood cell having a round nucleus |
| Phycoerythrin (PE) | An intensely bright phycobiliprotein isolated |
| | from red algae. It has excitation/emission |
| | spectrum peak wavelengths of 488-561/578 nm. |
| TVC | Total Viable Cells. TVC represents the number |
| | of viable cells in a given sample. TVC is |
| | calculated with results from NucleoCounter. |
| V500 | An organic dye with excitation/emission |
| | spectrum peak wavelengths of 415/500 nm. |

### II. Overview

In many aspects of manufacturing, developing tools for design improvement, quality control, etc. technologies may exist for identifying, reducing, and/or eliminating issues leading to production of non-commercializable product and delays. The technologies described herein reduce cost and delays in T cell manufacturing by improving product quality. However, development of said technologies may be laborious for a variety of reasons, especially, when the product includes living cells comprising machinery executing innumerable processes.

That said, living organisms are complex and often require development of complex support technologies. For example, the food industry may have an incentive to understand microbiology as it relates to food contamination and as such development around this technical area progresses.

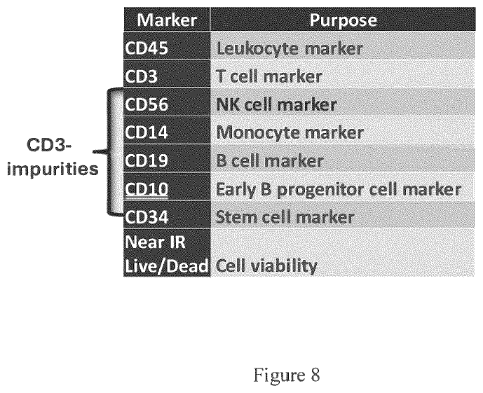

In the T cell and CAR T cell industries, living cells must be correctly modified and have their populations expanded. There is a need in the industry to characterize the population of cells at any point between collection from patient to infusion back into patient. Importantly, there is a need to characterize the population of cells going into a final product bag. The cell therapy industry needs to develop tools examining the cellular and molecular scale. FIG. 8 is a chart showing markers that relate to identifying a subpopulation of cells, according to various embodiments. The technologies described herein primarily relate to identifying and quantifying the “impurities” of FIG. 8 in a biological sample undergoing a cell therapy manufacturing process. In various embodiments, the term “impurities” may refer to “CD3 impurities” and/or “T cell impurities” and typically refer to leukocytes that are positive for a CD45 marker but include cells other than T cells.

The technology described herein includes marker panels, wherein each marker works in conjunction with the other markers to characterize a biological sample, according to various embodiments. In various embodiments, the disclosure provides methods, kits, and compositions for flow cytometric quantitation of CD3− cellular impurities in lymphocyte-rich samples. In various embodiments, the disclosure provides lit-for-purpose 2-8 color T-cell impurity flow cytometry panels of antibodies. In various embodiment, one or more of the antibodies described in those panels may be combined into a cocktail of antibodies for identifying CD3− cell impurities in a biological sample (e.g., a T cell sample). In various embodiments, the antibody cocktail may be lyophilized. In various embodiments, the disclosure provides methods of using the panels for the detection and quantification of CD3− cells in samples obtained at different stages of manufacturing of a T cell product for immunotherapy.

In various embodiments, the disclosure provides methods that may be used to identify, quantify, and optionally isolate, a variety of specific cell types using their cell surface marker pattern. These include, but are not limited to leukocytes, T cells, natural killer (NK) cells, natural killer T-cells (NKT cells), monocytes, B cells, early B progenitor cells, and stem cells. In various embodiments, the presence or absence of seven or more cell surface markers is determined simultaneously. In various embodiments, fluorescence activated cell sorting (FACS) analysis may be carried out all at once on a population of cells and it is possible to determine all at once what cells are present or absent based on their cell surface markers. In various embodiments, the method further assesses the cells' viability simultaneously with the cell surface markers. In various embodiment, it may not be necessary to run the FACS analysis more than once or with multiple samples in order to be able to characterize the cell impurities in a T cell product.

In various embodiments, the method provides for the detection and/or quantification of the total amount of T lymphocytes in a sample. In various embodiments, the method provides for the detection and quantification of the total amount of non-T lymphocytes in the same sample.

In various embodiments, the sample may be a blood sample from either a healthy donor or a patient (e.g., a cancer patient). In various embodiments, the sample may be an apheresis sample. In various embodiments, the sample may be from bone marrow. In various embodiment, the sample may comprise a commercially available mixture of blood cells such as CYTO-TROL™, Stem-Trol, Pan T cells, CD56+ NK cells, ALL patient's apheresis, NHL patients' apheresis among others. In various embodiments, a sample may comprise a biomarker-based standard. In various embodiments, the sample may be obtained from the manufacturing process for a T cell product for immunotherapy. In various embodiments, the T cell product may be a chimeric antigen receptor CAR-T cell product. In various embodiments, the sample may be obtained after enrichment of the apheresis product in T cells by density gradient separation. In various embodiments, the sample may have been obtained after enrichment of the apheresis product in CD4+ and/or CD8+ T cells by magnetic bead cell separation. In various embodiments, the sample is obtained from the end product ready for administration for immunotherapy.

In various embodiments, the method provides for the detection and quantification of the specific combination of cell populations identified in Tables 2A and 2B, or subcombinations thereof (at least two, at least three, at least four, at least 5, at least 6, at least 7). In various embodiments, the specific combination or subcombination of cells is identified by the specific combination or subcombination (e.g., CD45, CD10. CD19) of markers described in Table 2. In various embodiments, these markers are further combined with CD8 and CD4. Note that there are other possible cell surface markers that may be used to characterize “contaminating” cells in otherwise enriched lymphocyte compositions (e.g., CD25, CD2, CD7, and CD5). In various embodiments, the disclosure further provides a method to more specifically identify various types of cancer cells that may be present in a T cell population, where the T cell population is obtaining from an Acute Lymphocytic Leukemia (ALL) or Non-Hodgkin Lymphoma (NHL) patient. In various embodiment, Table 2 shows an exemplary specific combination of markers that is described in this application.

TABLE 2A
Exemplary Selection of Cell Surface Markers and Associated Parameters

| Antigen | Reporting Parameter Phenotype | Key Reporting Parameter/Unit |
|---|---|---|
| CD45 | Live/Singlet/Total Leukocyte/CD45⁺ | % CD45⁺ of Total Leukocytes |
| | Live/Singlet/Total Leukocyte/CD45^{dim} | % CD45^{dim} of Total Leukocytes |
| CD3 | Live/Singlet/Total Leukocyte/CD45⁺/ | % CD3⁺ T cells of Total |
| | CD14⁻/CD56⁻/CD3⁺ | Leukocytes |
| | Live/Singlet/Total Leukocyte/CD45⁺/ | % CD3⁻ non-T cells of Total |
| | CD14⁻/CD3⁻ | Leukocytes |
| CD56 | Live/Singlet/Total Leukocyte/CD45⁺/ | % CD56⁺CD3⁺ NKT cells |
| | CD14⁻/CD3⁺CD56⁺ | of Total Leukocytes |
| | Live/Singlet/Total Leukocyte/CD45⁺/ | % CD56⁺CD3⁻ NK cells |
| | CD14⁻/CD3⁻/CD56⁺ | of Total Leukocytes |
| CD14 | Live/Singlet/Total Leukocyte/CD45⁺/ | % CD14⁺ monocyte of Total |
| | CD14⁺ | Leukocytes |
| | Live/Singlet/Total Leukocyte/ | % CD14⁺ CD56⁺ cells of |
| | CD45⁺/CD14⁺CD56⁺ | Total Leukocytes |
| CD19 | Live/Singlet/Total Leukocyte/CD45⁺/ | % CD19⁺ B cells of Total |
| | CD14⁻/CD3⁻CD56⁻/CD19⁺ | Leukocytes |
| | Live/Singlet/Total Leukocyte/CD45⁺/ | % CD19⁺ CD34⁺ B progenitor |
| | CD14⁻/CD3⁻CD56⁻/CD19⁺CD34⁺ | cells of Total Leukocytes |
| | | % CD19⁺ CD34⁺ B cells of |
| | | CD3⁻ |
| CD34 | Live/Singlet/Total Leukocyte/CD45⁺/ | % CD34⁺CD19⁺ of Total |
| | CD14⁻/CD3⁻CD56⁻/CD19⁻/CD34⁺ | Leukocytes |
| | | % CD34⁺CD19⁺ of CD45^{dim} |
| | Live/Singlet/Total Leukocyte/CD45⁺/ | % CD34⁺CD19⁺ B progenitor |
| | CD14⁻/CD3⁻CD56⁻/CD19⁺CD34⁺ | cells of Total Leukocytes |
| | | % CD34⁺CD19⁺ B progenitor |
| | | cells of CD3⁻ |
| | Live/Singlet/Total Leukocyte/ | % CD34⁺CD19⁺ of Total |
| | CD45^{dim}/CD34⁺CD19⁺ | Leukocytes |
| | | % CD34⁺CD19⁺ of CD45^{dim} |
| | Live/Singlet/Total Leukocyte/ | % CD34⁺CD10⁺ of Total |
| | CD45^{dim}/CD34⁺CD10⁺ | Leukocytes |
| | | % CD34⁺CD10⁺ of CD45^{dim} |
| CD10 | Live/Singlet/Total Leukocyte/ | % CD10⁺CD19⁺ of |
| | CD45^{dim}/CD10⁺CD19⁺ | Total Leukocytes |
| | | % CD10⁺CD19⁺ of CD45^{dim} |

⁺means the cells display detectable levels of the marker.
⁻means the cells do not display detectable levels of the marker. dim means the cells display dim levels of the marker.

TABLE 2B
Exemplary Selection of Cell Surface
Markers and Associated Parameters

| | | Fluorophore/ | |
|---|---|---|---|
| Antibody | Clone | Conjugate | Purpose |
| CD3 | SK7 | APC | Pan T cell marker |
| CD10 | HI10a | FITC | Common ALL antigen; early B |
| | | | progenitor cell marker |
| CD14 | MφP9 | PerCP-Cy5.5 | Monocyte marker |
| CD19 | HIB19 | PE-Cy7 | B cell marker |
| CD34 | 561 | BV421 | Stem and progenitor cell marker |
| CD56 | NCAM16.2 | PE | NK cell marker |
| CD45 | HI30 | V500 | Pan leukocyte marker |
| Live/Dead | NA | APC-CY7/ | Cell Viability |
| Dye | | Near-IR dye | |

In various embodiments, CD4+ T cells may be identified as CD3+CD4+CD45+ cells. In various embodiments, CD8+ T cells may be identified as CD3+CD8+CD45+ cells. In various embodiments, CD45 may be used for the detection of CD45+ leukocytes as well as to differentiate CD45dim B-blasts from CD45+ population. In various embodiments, CD3 may be used to differentiate CD3+ T cells from CD3− non-T cells. In various embodiments, CD56 may be used to differentiate CD56+CD3+NK T cells and CD56+CD3− NK cells. In various embodiments, CD14 may be used to identify general CD14+ monocytes and aberrant cells co-expressing CD56 and/or CD34 antigen. In various embodiments, CD34 may be used to differentiate immobilized CD34+ cells in periphery, CD34+CD19+ and CD19− B-blast cells. In various embodiments, CD19 may be used to differentiate normal and aberrant CD19+ B cells expressing CD34 and/or CD10 surface antigen. In various embodiments, CD10 may be used to differentiate aberrant CD19+ early stage B progenitor cells or CD10+ immature B cells. In various embodiments, CD56+CD3− and CD56+CD3+ cells may be generally defined as NK and NKT cells respectively as CD56 antigen may be traditionally considered a NK cell marker in the hematopoietic system. However, it may be worth noting that CD56 expression has been reported to be not limited to NK or NKT cells, but also on other blood cells such as γδ T cells, αβ T cells and dendritic cells.

In various embodiments, the disclosure provides a method wherein each of these markers may be recognized by an antibody that may be fluorescently labeled with a different fluorochrome. In various embodiments, the antibody may be a polyclonal antibody. In various embodiments, the antibody may be a monoclonal antibody.

Multicolor flow cytometry, as opposed to single-color flow cytometry, introduces a higher technical difficulty in assay development. To analyze several surface markers simultaneously, each surface marker requires a specific antibody for detection. In flow cytometry it is best to use antibodies directly conjugated to fluorochromes instead of primary antibodies for detection and secondary antibodies for signal amplification. Therefore, when using multiple antibodies simultaneously, their conjugated fluorochromes must be chosen wisely so that they do not overlap in their emitted wavelengths. Fluorochromes that are as far apart as possible in the color spectra may be chosen. Panel selection depends on multiple factors including accurate compensation and antigen-fluorochrome balancing.

In various embodiments, each antibody may be labeled with a different fluorochrome/fluorophore. In various embodiment, the fluorochromes may be selected from any fluorochrome known in the art based on, for example, the relative abundance of the cell surface marker on the surface of the cells and the relative fraction of the cell population that each cell type represents.

In various embodiments, the fluorophore brightness increases in the order V500, near-IR dye (lowest); APC-Cy7, PerC.P-Cy5.5; FITC; PE-Cy7; BV421, APC; PE, PE-Cy7 (highest). In various embodiments, the antigen abundance and/or density decreases in the order of CD45+(highest); CD3+; CD14+, CD19+; CD56+; CD10+; and CD34+(lowest). Control purified Pan-T cells, human peripheral blood CD19+ B cells, human peripheral blood NK cells, and other purified cells are available in the art from different manufacturers (e.g., StemCell Technologies™).

Strategically, antigens in higher abundance may be matched with dimmer flurochromes whereas those antigens with low abundance are matched with brighter fluorochroms. There may be various industry standards known to one of ordinary skill in the art.

In various embodiments, the fluorochromes may be selected from any fluorochrome, including V500 (or any other blue emission dye), FITC (or any other green emission dye), BV421 (or any other blue emission dye), PE (or any other yellow emission dye), APC (or any other red emission dye), PE-Cy7 (or any other far red emission dye), PerCP.Cy5.5 (or any other far red emission dye), PacificBlue (or any other blue emission dye), PerCP (or any other red emission dye, any AlexaFluor (e.g., AlexaFluor700 (or any other red emission dye), AlexaFluor647 (or any other red emission dye), V450 (e.g., BD Horizon V450, or any other blue emission dye), APC-Cy7 (or any other infrared emission dye), SAV-TR-PE, PE-Cy7 (or any other infrared emission die), PE-Texas Red, Texas Red (or any other orange emission dye), AmCyan (or any other green emission dye), Alexa Fluor 488 (or any other green emission dye), PE-Cy5 (or any other red emission dye), DyeCycle dyes, Fluo-3, Fluo-5, Fura dyes, Qdot dyes, FVS dyes, Sytox dyes, and any other fluorescent dyes available in the art. In various embodiments, the live/dead dye may be APC-CY7/Near-IR dye.

In various embodiment, one or more of the fluorescently-labeled antibodies may be selected from the antibodies in Table 3.

TABLE 3
Exemplary Fit-for-Purpose Antibody Panels

| Antigen | CD45 | CD10 | CD34 | CD56 | CD3 | CD19 | CD14 |
|---|---|---|---|---|---|---|---|
| Exemplary | V500 | FITC | BV421 | PE | APC | PE-Cy7 | PerCP.Cy5.5 |
| Fluorochrome | | | | | | | |
| Other | ANY | ANY | ANY | ANY | ANY | ANY | ANY |
| Fluorochromes | | | | | | | |

In various embodiment, the fluorochromes are distributed differently than in the specific allocation in Table 3. For example, in one embodiment, the anti-CD45 antibody is FITC-labeled and the anti-CD10 antibody is V500 labeled. In various embodiments, at least one of the antibody labels may be selected from other fluorescent labels available in the art. In various embodiments, at least one of the antibodies that may be used to identify the cells in the sample may not be from Table 3.

In various embodiments, each of the anti-CD45, anti-CD10, anti-CD34, anti-CD56, anti-CD3, anti-CD19, and anti-CD14 antibodies may be custom made. In various embodiments, any of these antibodies may be selected from any commercially available antibody against these cell surface markers. There may be numerous commercially available antibodies against these marker antibodies, which may be acquired from, for example, BD Biosciences, Abcam, Thermofisher Scientific Inc.™, Sinobiological™, Biolegend™, R&D Systems™, Sigma Aldrich™, Stem Cell™, Santa Cruz Biotechonologies™, ProteinTech™, or any other antibody provider. In one embodiment, one or more antibodies is selected from the antibodies in Table 4.

TABLE 4
Exemplary Clones for a Fit-for-Purpose Panels

| Antigen | CD45 | CD10 | CD34 | CD56 | CD3 | CD19 | CD14 |
|---|---|---|---|---|---|---|---|
| Exemplary | HI30 | H10a | 561 | NCAM16.2 | SK7 | HIB19 | MφP9 |
| Choice | | | | | | | |
| Other | ANY | ANY | ANY | ANY | ANY | ANY | ANY |
| Clones | | | | | | | |

In various embodiments, the anti-CD19 antibody may be selected from clones SJ25C1 and HIB19. In various embodiments, the anti-CD14 antibody may be selected from clones MφP9 and M5E2. In various embodiments, the anti-CD56 antibody may be selected from clones NCAM16.2 and HCD56. In various embodiments, the specificity for CD34, CD19, and CD56 conjugated antibodies may be examined by testing known positive and negative samples for the corresponding markers. In a non-limiting example, for CD34 antibody specificity, Stem-Trol™ (commercially sourced/manufactured CD34+ positive control cells) from StemCell Technologies™ may be used as the positive sample. In some embodiments, MAVER-1/MRL3008 (CD19+ B cell line), and pure NK cells (from StemCell Technologies™) may be used as positive samples for the specificity of CD19 and CD56 antibodies, respectively. CD34+ cells, CD19+ cells and NK cells percentages are the output measurements for this assessment. The positive control testing material, CYTO-TROL, may also used in the specificity test, as it has lot-specific reference ranges provided by the manufacturer. In various embodiments, the accuracy and other performance parameters of a method that uses one or more antibodies other than those in Tables 4-6 may be assessed by using the methods as reference values. The linearity of each assay may be determined using serial dilutions as per established methods.

In various embodiments, one or more antibodies is selected from the antibodies in Table 5.

TABLE 5
Exemplary Clone / Fluorochrome Combination for Fit-for-Purpose Panels

| Antigen | CD45 | CD10 | CD34 | CD56 | CD3 | CD19 | CD14 |
|---|---|---|---|---|---|---|---|
| Clone | HI30 | H10a | 561 | NCAM16.2 | SK7 | HIB19 | MφP9 |
| Fluorochrome | V500 | FITC | BV421 | PE | APC | PE-Cy7 | PerCP.Cy5.5 |

Panel selection also depends on optimally titrated antibodies. In various embodiments, one or more of the antibodies and their respective amounts in a staining composition may be selected from those of Table 6. In various embodiments, the composition, also described herein as an antibody cocktail, has been lyophilized.

TABLE 6
Exemplary Amounts for Exemplary Fit-for-Purpose Panels for 1 × 10⁶ Cells

| Antigen | CD45 | CD10 | CD34 | CD56 | CD3 | CD19 | CD14 |
|---|---|---|---|---|---|---|---|
| Clone | HI30 | H10a | 561 | NCAM16.2 | SK7 | HIB19 | MφP9 |
| Fluorochrome | V500 | FITC | BV421 | PE | APC | PE-Cy7 | PerCP.Cy5.5 |
| Antibody | 0.20 | 0.52 | 0.5 | 0.02 | 0.05 | 0.05 | 0.065 |
| μg/Test | | | | | | | |
| Antibody/ | 2.0 μL | 1.3 μL | 5.0 μL | 1.3 μL | 1.0 μL | 1.0 μL | 1.3 μL |
| μl/Test* | | | | | | | |

*total 12.9/100 μl, plus, optionally, 87.1 μl of staining buffer and 200 μl of LIVE/DEAD Near-IR Fixable Dye at 1:3000 dilution.

In various embodiment, the total amount of each antibody may be different from those in Table 6. In various embodiment, the ratio of each antibody in the fit-for-purpose product is as reflected in Table 7.

TABLE 7
Exemplary Ratios (%) of Antibody in the Staining Composition / Product,
Relative to the Total Amount of Antibody in the Product

| Antigen | CD45 | CD10 | CD34 | CD56 | CD3 | CD19 | CD14 |
|---|---|---|---|---|---|---|---|
| Clone | HI30 | H10a | 561 | NCAM16.2 | SK7 | HIB19 | MφP9 |
| | or | or | or | or | or | or | or |
| | other | other | other | other | other | other | other |
| Fluorochrome | V500 | FITC | BV421 | PE | APC | PE-Cy7 | PerCP.Cy5.5 |
| | or | or | or | or | or | or | or |
| | other | other | other | other | other | other | other |
| Antibody | About | About | About | About | About | About | About |
| (% total**) | 1.6% | 4.0% | 4.0% | 0.16% | .41% | .41% | 0.5% |
| Antibody | About | About | About | About | About | About | About |
| (% total^{##}) | 8.5% | 34.0% | 32.7% | 1.3% | 3.3% | 3.3% | 17.0% |
| Antibody | About | About | About | About | About | About | About |
| (% total^{%%}) | 8.5% | 34.0% | 32.7% | 1.3% | 3.3% | 3.3% | 17.0% |
| Antibody | 8.5% | 34.0% | 32.7% | 1.3% | 3.3% | 3.3% | 17.0% |
| (% total) | | | | | | | |

**“about” means within 1 standard of deviation
^{##“}about” means plus or minus 10% of the recited number
^{%%“}about” means plus or minus 20% of the recited number

In various embodiments, one or more of the antibodies may be present in an amount that is 1%, 5%, 10%, 15%, 20%, 25%, 30%, 35%, 40%, 45%, 50%, 55%, 60%, 65%, 70%, 75%, 80%, 85%, 90%, 95%, 100%, 150%, 200%, 250%, 300%, 350%, 400%, 450%, etc., or fractions thereof more than or less that the amounts in Table 6. In various embodiments, the ratio of CD10, CD34, CD56, CD3, CD19, and/or CD14 antibody changes in the staining composition/product/cocktail, relative to the amount of CD45 antibody. In various embodiments, the ratio of CD45, CD34, CD56, CD3, CD19, and/or CD14 antibody may change, relative to the amount of CD10 antibody. In various embodiments, the ratio of CD45, CD10, CD56, CD3, CD19, and/or CD14 antibody may change, relative to the amount of CD34 antibody; and so on and so forth. In various embodiments, the ratios may change because the fluorochrome changes thereby changing the number of moles of antibody per microgram relative to those of Table 6. In various embodiments, the fluorochrome may change but the ratio of antibodies in terms of moles of unlabeled antibody may stay the same as that shown in Table 6.

In various embodiments, the total amount of antibody per test may be that in Table 6. In various embodiments, the amount of each individual antibody per test may be independently increased or decreased by 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 96, 97, 98, 99, 100, 200, 300, 400, or 500 percent, or fractions thereof, relative to the amounts in Table 6. In various embodiments, the amount of each individual antibody per test may be independently increased or decreased by 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 96, 97, 98, 99, 100, 200, 300, 400, or 500 fold, or fractions thereof, relative to the amounts in Table 6.

In various embodiments, the amount of each of the antibodies per test may be independently increased or decreased by 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 96, 97, 98, 99, 100, 200, 300, 400, 500, 600, 700, 800, 900, 1000 nanograms, or fractions thereof, relative to the amounts in Table 6.

In various embodiments, the amount of each of the antibodies per test may be independently increased or decreased by 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 96, 97, 98, 99, 100, 200, 300, 400, 500, 600, 700, 800, 900, 1000 micrograms, or fractions thereof, relative to the amounts in Table 6.

In various embodiments, to optimize the antibody panels, antibodies may be titrated to determine the use volume/concentration that gives a robust signal-to-noise ratio, minimum background, and staining intensity with consistent percentage positive signal. In various embodiments, in order to determine the optimal concentration for the staining, antibodies may be serially diluted and the stain index (SI) calculated as [MFIp-MFin]/2×rSDn, where MFIp is median fluorescence intensity (MFI) for the positive population, MFIn is MFI for the negative population, and rSDn is robust standard deviation of the negative population. In various embodiments, this is done by a method described in Maecker HT. et al. Cytometry Part A 2006 (69A): 1037-1042. In various embodiments, a plot of SI may be created to select the robust mass of antibodies that gives significant SI values. Excess antibody volume may artificially increase both the positive and negative signal of the entire cell population.

In various embodiments, fewer than all seven antibodies described in the Tables above may be used in the method of identifying CD3− impurities in a T cell sample and/or are mixed in the staining composition or cocktail. In various embodiments, the cocktail comprises only antibodies to detect CD45+CD3+ lymphocytes (all lymphocytes in a mixture). In various embodiments, the cocktail comprises only antibodies to detect NK T cells, which are CD45+/CD3+/CD56+. In various embodiments, the cocktail comprises only antibody to detect NK cells, which are CD45+/CD3−/CD56+. In various embodiments, the cocktail comprises only antibodies to detect monocytes, which are CD45+/CD3−/CD14+CD19−. In various embodiments, the cocktail comprises only antibodies to detect B cells, which are CD45+/CD3-/CD14-CD19+. In various embodiments, the cocktail comprises only antibodies to detect stem and progenitor cells, which are CD45+/CD34+. In various embodiments, the cocktail comprises only antibodies to detect early B progenitor cells, which are CD45dim/CD10+CD19+. In various embodiments, the cocktail comprises antibodies for any combination thereof. In various embodiments, the cocktail may be lyophilized.

In various embodiments, the antibody cocktail composition comprises enough antibodies for a pre-determined number of tests (each test being the contacting of a population of cells with the cocktail of antibodies). In various embodiments, the total volume of antibody cocktail/mixture per test sample may be 100 μL. In various embodiments, the total volume of antibody cocktail/mixture per test sample may be 10 μL, 50 μL, 100 μL, 200 μL, 300 μL, 400 μL, 500 μL, 600 μL, 700 μL, 800 μL, 900 μL, or 1000 μL. In various embodiments, the total volume of antibody cocktail/mixture per test sample may be 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 96, 97, 98, 99, or 100 μL(s).

In various embodiments, each test may be designed to analyze approximately 1 million blood cells. In various embodiments, each test may be designed to analyze approximately 2 million, 3 million, 4 million, 5 million, 6 million, 7 million, 8 million, 9 million, or 10 million cells. In various embodiments, the cell sample may be a volume of approximately 200 μL. In various embodiments, the cell sample may have a volume of approximately 10 μL, 50 μL, 100 μL, 200 μL, 300 μL, 400 μL, 500 μL, 600 μL, 700 μL, 800 μL, 900 μL, or 1000 μL. In various embodiments, the cell sample comprises 1 million cells in 200 μL of cell staining buffer. In various embodiments, each sample comprises approximately 1 million cells in 200 μL of cell stain buffer and this may be mixed with 100 μL of antibody mixture for analysis.

In various embodiments, the disclosure provides a container carrying enough of a cocktail/mixture of the seven antibodies of the above tables for 20 samples. In various embodiments, the container carries enough antibody mixture/cocktail for staining 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 96, 97, 98, 99, or 100 samples per container. In various embodiments, the mixture/cocktail is lyophilized. In various embodiments, the mixture/cocktail is suspended in a buffer.

In various embodiments, the lyophilized cocktail (for example, the amounts specified in Table 6 or Table 7) is resuspended in a buffer appropriate for use in FACS. In various embodiments, the resuspension may be stable for at least 10 days at room temperature, when resuspended in 2000 μL of buffer. In various embodiments, the resuspension may be stable for at least 3 months at room temperature, when resuspended in 400 μL of buffer. In various embodiments, the resuspension may be stable for at least or approximately 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 96, 97, 98, 99, or 100 days at room temperature when resuspended in 100, 200, 300, 400, 500, 600, 700, 800, 900, 1000, 1100, 1200, 1300, 1400, 1500, 1600, 1700, 1800, 1900, or 2000 μL(s) of buffer.

In various embodiments, the assay as a lower limit of quantitation (LLOQ) of each of the CD3− populations (e.g., CD34+, CD56+NK, CD19+ B cells) of about 0.2% for CD34+ cells and CD19+ B cells and about 1.4% for CD56+CD3− NK cells. In various embodiments, the LLOQ may be about 0.1, 0.2. 0.3, 0.4, 0.5, 0.6, 0.7, 0.8, 0.9, 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, or 50%. In various embodiments, the LLOQ may be assessed as a linearity study by mixing target population with a negative population. Serial dilutions may be made by a factor of 2 (6.25%, 3.13%, 1.56%, 0.78%, 0.39%, 0.2%, 0.1%, 0.05%, 0.02%, 0.01% and 0.00%), and each dilution may be tested in triplicate. The lowest dilution with the acceptable % Recovery (within 80% to 120%) and the acceptable % CV for replicates (<25) may be set as the LLOQ. In various embodiments, a LLOQ test may be performed to confirm if the assay is sensitive enough to detect CD34+ populations below 10%. CD34+ cells are typically rare in human PBMCs.

In various embodiments, the sample may comprise an apheresis sample comprising healthy donor PBMC. The typical cellular composition of such sample comprises 25-60% CD4+ T cells, 5-30% CD8+ T cells, 5-10% CD19+ B cells, 10-30% CD56+CD3− NK cells, and 4-10% CD14+ monocytes. In various embodiments, the sample may comprise an apheresis sample comprising PBMC from a cancer patient.

In various embodiments, the disclosure provides a method of characterizing CD3− cells (e.g., NK-T cells, NK cells, monocytes, early B cell progenitor cell, or combinations thereof), which may be considered impurities, in a T cell preparation comprising contacting a sample of the T cell preparation with a cocktail of antibodies as described in this disclosure and analyzing the mixture for the distribution of cells with specific cell surface markers by fluorescence detection methods.

In various embodiments, the disclosure provides a method of treating cancer in a subject in need thereof with a T cell preparation wherein one or more of the CD3− impurities (e.g., NK-T cells, NK cells, monocytes, early B cell progenitor cell, or combinations thereof) in the T cell preparation have been or may be characterized by a method that requires the use of one or a mixture/cocktail of antibodies as described in this disclosure. In various embodiments, the T cell preparation may be autologous. In various embodiments, the T cell preparation may be allogeneic. Examples of T cell populations and of methods of preparation of exemplary T cell populations for immunotherapy are described herein. In various embodiments, the T cells may be engineered with a CAR or T cell receptor. Examples of CARs and T cell receptors are described herein.

In various embodiments, the disclosure provides a method for determining whether a T cell product is suitable for immunotherapy, comprising characterizing one or more of the CD3− cell impurities (e.g., NK-T cells, NK cells, monocytes, early B cell progenitor cell, or combinations thereof) in the T cell product using one of the antibodies or cocktail of antibodies described in this disclosure and determining whether the T cell product is suitable based on the levels of CD3− cell impurities in the T cell product. In various embodiments, the acceptable levels may be set by regulatory authorities (e.g., FDA, EMEA, etc.). In various embodiments, the levels of at least one of the cell types is above accepted levels. In various embodiments, the levels of at least one of the cell types is below accepted levels.

In various embodiments, the disclosure provides a method/assay or a kit for identifying at least one of leukocytes, NK-T cells, NK cells, monocytes total lymphocytes, early B cell progenitor cell, or combinations thereof in blood cell populations. In various embodiments, the assay and/or kit is used to characterize CD3− cells in T cell products for immunotherapy. In various embodiments, the kit comprises (a) one of more antibodies to detect one or more cell markers for any one or more of these cells (see, e.g., Table 2) and (2) reagents to carry on the binding of the antibody with the cell surface markers, and, optionally, (3) instructions for using the reagents for the kit's purpose. In various embodiments, the antibodies may be lyophilized together in the same container (e.g., a Lyovial). In various embodiments, the antibodies may be selected from Table 3. In various embodiments, the antibodies may be selected from Table 4. In various embodiments, the antibodies may be selected from Table 5. The amounts of each antibody in the vial(s) of the kit may vary from these amounts, as described elsewhere in the specification.

All publications, patents, patent applications and other documents cited in this application are hereby incorporated by reference in their entireties for all purposes to the same extent as if each individual publication, patent, patent application or other document were individually indicated to be incorporated by reference for all purposes.

While various specific embodiments have been illustrated and described, it will be appreciated that various changes may be made without departing from the spirit and scope of the disclosure.

### III. Exemplary Biological Samples

In various embodiments, biological samples may comprise, originate from, or may be derived from blood (e.g., apheresis). In various embodiments, biological samples may originate from or may be derived from a biological fluid (e.g., intestinal fluid, chyme, cerebrospinal fluid, phlegm, synovial fluid, mucus, bile, pus, saliva, lymph, blood plasma). In various embodiments, biological samples may comprise a product or a component thereof. For example, in various embodiments, biological samples may comprise one or more immunotherapy products and/or T cell therapy products. In various embodiments, biological samples may be CAR T cell therapy products.

The technology described herein, and specifically pertaining to biomarker-based standards and uses thereof, may be applicable to a variety of disciplines and may be well suited to analyzing biological samples comprising cell populations. In various embodiments, cell populations may comprise leukocyte populations. In various embodiments, leukocyte populations may comprise monocytes, B cells, NK cells, NKT cells, T cells, early progenitor cells, and step cells, or any combination thereof.

More specifically, non-limiting examples of biological samples comprising cell populations may include cell therapy products, cell therapy product inputs, or cell therapy manufacturing intermediates for cell therapy manufacturing.

In various embodiments, suitable samples or starting material may comprise an apheresis or an apheresis material from a patient.

In various embodiments, suitable samples may include a T cell therapy final product and/or a CAR T cell therapy final product. In various embodiments, characterizing a cell population of a final product may be useful in determining whether the final product meets a product specification.

In various embodiments, samples may be collected and analyzed at any point along a CAR T cell manufacturing process. In various embodiments, a sample may comprise an apheresis material or another a starting material collected from a donor. In various embodiments, apheresis may be transported and processed into a cell therapy product.

In various embodiments, samples may be collected and analyzed after an activation step, a transduction step, an expansion step, or pre or post any other manufacturing step or combination thereof. In various embodiments, results at various points along a manufacturing process may be compared to collect data regarding a protocol. In various embodiments, comparison data may be used to adjust a protocol and/or a manufacturing step.

In various embodiments, a method for characterizing a population of cells into subpopulations may comprise providing a biological sample comprising a population of leukocytes. In various embodiments, the population of leukocytes comprises a plurality of subpopulations. In various embodiments, the subpopulations comprise T cells, NK cells, monocytes, B cells, B progenitor cells, and stem cells.

### IV. Exemplary Compositions

In various embodiments, compositions may comprise a population of biomarker-based standards. The biomarker-based standards described herein serve a number of purposes. In various embodiments, a purpose includes quantifying a total leukocyte population. In various embodiments, a purpose includes quantifying subpopulations of a leukocyte population in comparison to a total leukocyte population. A major technical problem solved by the biomarker-based standards described herein is the ability to characterize all leukocyte subpopulations. In various embodiments, subpopulations may resemble those found in healthy donors. In various embodiments, subpopulations may resemble those found in clinical patients. In various embodiments, subpopulations may resemble those found in both healthy donors and clinical patients.

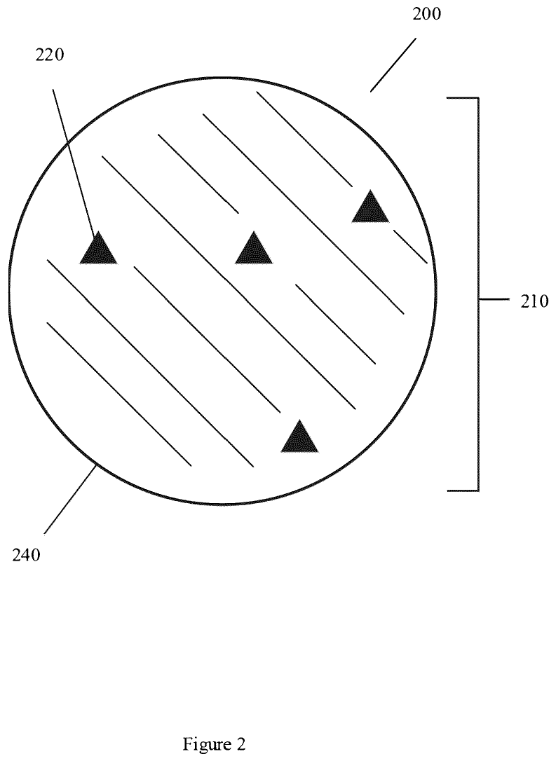

FIG. 2 is a cartoon illustration of a biomarker-based standard 200, according to various embodiments. In various embodiments, the biomarker-based standard 200 may comprise a scaffold 210 and one or more markers 220. In various embodiments, the biomarker-based standard 200 may comprise a surface 240. In various embodiments, the one or more markers 220 may be attached to the surface 240. In various embodiments, the one or more markers 220 may be embedded beneath the surface 240.

In various embodiments, scaffolds 210 may comprise simplified, cell-like structures. In various embodiments, the simplified, cell-like structures may have analogs to naturally occurring cells. In various embodiments, the cell-like structures may comprise markers 220, binding proteins, or anything capable of associating one or more markers 200 together in a spatially restricted configuration.

In various embodiments, the scaffold 210 may comprise one or more components that mimic cellular structures (e.g., fatty acids, nucleotides, amino acid structures, etc.) and/or behavior (e.g., metabolism, genetic cell surface marker expression, etc.). The terms “synthetic cell” and “artificial cell” may comprise the same or overlapping meaning. A biomarker-based standard may mimic specific behaviors of a cell such as those described herein and elsewhere. For a non-limiting example, in various embodiments, the specific behavior may include expression of one or more cell surface markers 220.

In various embodiments, the one or more markers 220 may comprise a marker set. In various embodiments, the marker set may comprise all the markers 220 necessary to detect a presence of a cell type (e.g., one or more of the populations or subpopulations of cells described herein). In various embodiments, the marker set may comprise all the markers 220 necessary to quantify a cell type. In various embodiments, markers may be strategically placed with other markers included on the same or similar cell types (e.g., see marker sets described herein).

In various embodiments, a composition for characterizing a population of cells into subpopulations may comprises a population of biomarker-based standards 200.

In various embodiments, each biomarker-based standard 200 may comprise a common marker. In various embodiments, the common marker may correspond to a marker associated with leukocytes. In various embodiments, a common marker may be used to recognize a genus population of cells (e.g., leukocytes). In various embodiments, the common marker may be present on all cell species to the genus.

In various embodiments, each biomarker-based standard 200 may comprise at least one of a plurality of marker sets, wherein the marker sets correspond to marker sets of T cells, NK cells, monocytes, B cells, early progenitor cells, and stem cells. In various embodiments, the T cells, NK cells, monocytes, B cells, early progenitor cells, and stem cells may be a plurality of species cell types among a larger genus of cells.

In various embodiments, each biomarker-based standard 200 may comprise a scaffold 210 for restricting relative movement between the common marker and the at least one marker set. In accordance with various embodiments, a scaffold can be nearly anything. Non-limiting examples of scaffolds commonly occurring in nature comprise, living cells, micelles, phospholipid monolayers, phospholipid bilayers.

In various embodiments, the common marker may comprise CD45, wherein CD45 may be comprised of populations of CD45hi and CD45dim. In various embodiments, CD45 may be a common marker for a genus of leukocytes. In various embodiments, CD45hi may be present on a single scaffold. In various embodiments, CD45dim may be present on a single scaffold.

In various embodiments, a marker set of the plurality of marker sets may comprise CD10. CD19. CD45dim. In various embodiments, the combination of CD10, CD19, CD45dim may be present on a single scaffold.

In various embodiments, a marker set of the plurality of marker sets may comprise CD19, CD34, and CD45dim. In various embodiments, the combination of CD19. CD34, and CD45dim may be present on a single scaffold.

In various embodiments, a marker set of the plurality of marker sets may comprise CD3, CD14, and CD45. In various embodiments, the combination of CD14, and CD45 may be present on a single scaffold.

In various embodiments, a marker set of the plurality of marker sets may comprise CD3, CD45, and CD56. In various embodiments, the combination of CD3, CD45, and CD56 may be present on a single scaffold.

In various embodiments, a marker set of the plurality of marker sets may comprise CD3, CD19, and CD45. In various embodiments, the combination of CD3, CD19, and CD45 may be present on a single scaffold.

In various embodiments, the plurality of marker sets may comprise the plurality of marker sets comprise CD3, CD10, CD14, CD19, CD34, CD45, CD45dim, and CD56.

In various embodiments, at least some of the population of biomarker-based standards further comprise amine binding sites that react to Near IR viability dye. In various embodiments, between about 15-20% of the population of biomarker-based standards comprise amine binding sites. In various embodiments, between about 16.7-100% of the population of biomarker-based standards do not comprise amine binding sites.

In various embodiments, between about 50-60% of the population of biomarker-based standards comprise CD45hi. In various embodiments, between about 25.2-66.1% of the population of biomarker-based standards comprise CD45hi. In various embodiments, about 38.2% of the population of biomarker-based standards comprise CD45hi.

In various embodiments, about 40-50% of the population of biomarker-based standards comprise CD45dim. In various embodiments, about 32.3-74.7% of the population of biomarker-based standards comprise CD45dim. In various embodiments, about 59.7% of the population of biomarker-based standards comprise CD45dim.

In various embodiments, between about 50-60% of the population of biomarker-based standards comprise CD10. CD19, and CD45dim. In various embodiments, between about 29.7-66.7% of the population of biomarker-based standards comprise CD10, CD19, and CD45dim. In various embodiments, about 49.2 of the population of biomarker-based standards express CD10, CD19, and CD45dim.

In various embodiments, between about 50-60% of the population of biomarker-based standards comprise CD19, CD34, and CD45dim. In various embodiments, between about 0.3-68.2% of the population of biomarker-based standards comprise CD19, CD34, and CD45dim. In various embodiments, about 45.6% of the population of biomarker-based standards comprise CD19, CD34, and CD45dim.

In various embodiments, the markers may be embedded within the scaffolds. In various embodiments, the markers may be the markers are located on a surface of the scaffolds. In various embodiments, the scaffold may comprise a phospholipid bilayer. In various embodiments, the scaffold comprises a hydrogel, an aerogel, a cryogel, an organogel, a nanogel, a conductive hydrogel, or any combination thereof.

The fluorescent staining intensity may be determined by comparison of the negative control and the staining of each antibody. Negative staining may be defined by fluorescent intensity similar to that of a negative control. In general, positive staining may be categorized as dim, moderate or bright. Dim staining may be defined as slightly increased when compared to the negative control. Moderate staining may be defined as at least one log decade brighter than the negative control. Bright staining may be defined as at least two log decades brighter than the negative control.

In various embodiments, CD45hi (or just CD45) may correspond to blast cell populations in human T lymphoblastic leukemia.

In various embodiments, biomarker-based standard may comprise CD45, CD45dim, CD3, CD14. CD19. CD56. CD10, and CD34. In various embodiments, one or more sets of biomarker-based standard may comprise CD45, CD45dim, CD3, CD14, CD19, CD56, CD10, and CD34.

In various embodiments, a biomarker-based standard may comprise CD45. In various embodiments, a biomarker-based standard may comprise CD45 and CD14. In various embodiments, a biomarker-based standard may comprise CD45 and CD3. In various embodiments, a synthetic biomarker-based standard may comprise CD45 and CD56. In various embodiments, a biomarker-based standard may comprise CD45 and CD19. In various embodiments, a biomarker-based standard may comprise CD45dim, CD19, and CD10. In various embodiments, a biomarker-based standard may comprise CD45dim, CD19, and CD 34.

In various embodiments, a scaffold may comprise ActiveMax® Human BCMA μBeads.

In various embodiments, biomarker-based standards may comprise B cell markers. In various embodiments, the B cell markers may comprise CD5, CD20, CD22, CD33 and/or LIVE/DEAD IR876. In various embodiments, sets of markers may comprise any combination of CD5, CD20. CD22. CD33 and/or LIVE/DEAD TR876.

### V. Exemplary Detection Molecules and Labels

Detection molecules and labels have broad applicability in the life sciences and biopharma industries. Many of the compositions, kites, and methods described herein may be ideally suited for flow cytometry applications. However, a skilled artisan will appreciate that detection molecules and labels may be used in a variety of systems and devices. Non-limiting examples may include flow cytometers, cell counting devices, and thermocyclers executing polymerase chain reaction (PCT). A skilled artisan will appreciate that the detection molecules and labels described herein have broad applications, including but not limited to, cell based product applications, cell therapy manufacturing, and cell therapy quality control. In various embodiments, the selection of detectable cell surface markers and design of combination(s) of detection molecules 301 and labels 305 may optimize spectral overlap reduction (see Overview).

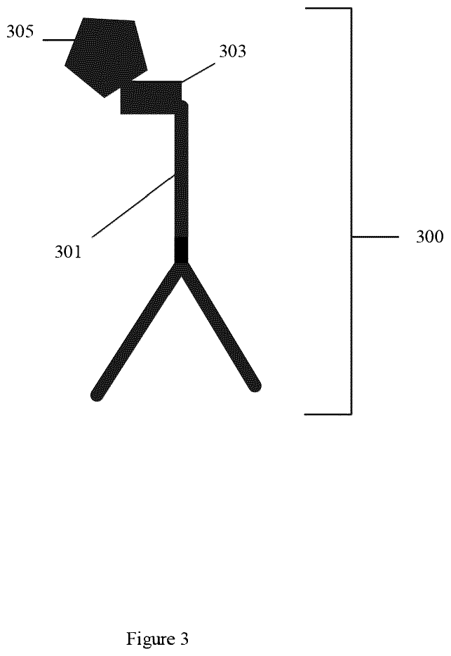

FIG. 3 is a cartoon illustration of labeled detection molecule 300, according to various embodiments. In various embodiments, a labeled detection molecule 300 may comprise a detection molecule 301 (e.g., an antibody). In various embodiments, a labeled detection molecule 300 may comprise a detection molecule 301 and a label 305 (e.g., a fluorochrome). In various embodiments, a linker 303 may be used to connect the detection molecule 201 and the label 305. In various embodiments, linkers may also be used to bind markers to scaffolds.

In various embodiments, detection molecules 301 may be used to detect the presence of one or more markers (e.g., one or more of the markers described herein).

When in use, one or more detection molecules 301 may be detected in close proximity, adjacent to, or within a cell (e.g., a leukocyte), according to various embodiments. In various embodiments, a combination or markers or marker sets may identify a cell's type. Each detection molecule may be specific for a specific marker (e.g., a CD3, a CD10, a CD14, a CD19, a CD34, a CD45, a CD45dim, a CD56, etc.), according to various embodiments.

In various embodiments, a labeled detection molecule 300 may bind to a target marker. In various embodiments, the identification may be based on which detection molecules 301 are detected on the cell. Of course, there must be a way to both observe and identify detection molecules 301. In various embodiments, a number of fluorochromes a suitable and allow differentiation between different biomarker-based standards and/or cells.

In various embodiments, the kits and methods described herein may use an antibody cocktail. In various embodiments, the antibody cocktail may comprise one or more labeled detection molecules 300. In various embodiments, the antibody cocktail may comprise an antibody for detecting the CD3. In various embodiments, the antibody cocktail may comprise an antibody for detecting the CD10. In various embodiments, the antibody cocktail may comprise an antibody for detecting the CD14. In various embodiments, the antibody cocktail may comprise an antibody for detecting the CD19. In various embodiments, the antibody cocktail may comprise an antibody for detecting the CD34. In various embodiments, the antibody cocktail may comprise an antibody for detecting the CD45/CD45dim. In various embodiments, the antibody cocktail may comprise an antibody for detecting the CD56.

In various embodiments, the antibody for detecting the CD3 may be conjugated to an APC fluorochrome. In various embodiments, the antibody for detecting the CD10 marker may be conjugated to a FITC fluorochrome. In various embodiments, the antibody for detecting the CD14 marker may be conjugated to a PerCP Cy5.5 fluorochrome. In various embodiments, the antibody for detecting the CD19 may be conjugated to a PE.Cy7 fluorochrome. In various embodiments, the antibody for detecting the CD34 may be conjugated to a BV421 fluorochrome. In various embodiments, the antibody for detecting the CD45/CD45dim marker may be conjugated to a V500 fluorochrome. In various embodiments, the antibody for detecting the CD56 marker may be conjugated to a PE fluorochrome.

Additional labeled detection molecules are described in International Patent Application WO2022/093925, U.S. Patent Publication No. US2022/0155299A1 and US2024/0168016A1 the disclosures of which are herein incorporated by reference in their entireties for all purposes.

### VI. Exemplary Methods of Characterizing Cell Populations

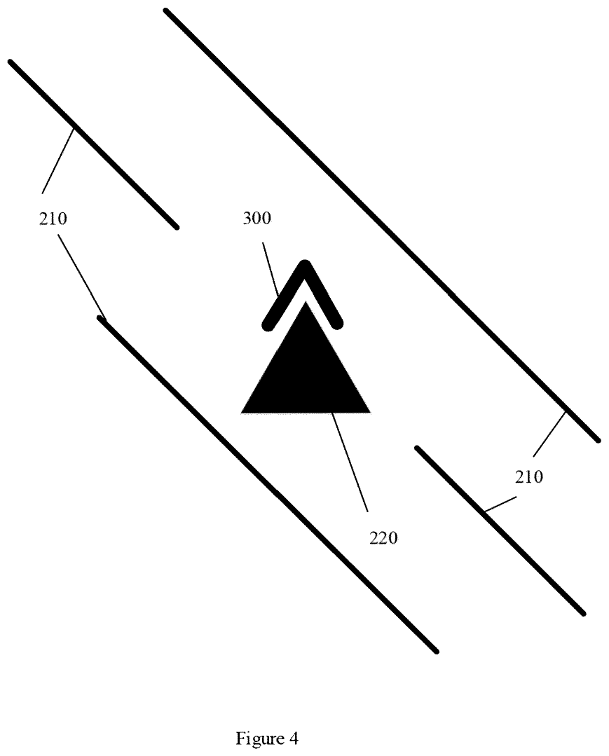

FIG. 4 is a cartoon illustration of a labeled detection molecule 300 bound to a marker 220 associated with a scaffold 210 of a biomarker-based standard 200, according to various embodiments. In various embodiments, FIG. 4 may represent a point in a process where biomarker-based standards may undergo analysis by a flow cytometer. In FIG. 4, an antibody cocktail may have already been added to a mixture, according to various embodiments and antibodies have bound to markers 220.

In various embodiments, the marker 220 may be embedded within the scaffolds 210. In various embodiments, the markers 220 may be located on a surface of the scaffolds 210. In various embodiments, the scaffold 210 may comprise a phospholipid bilayer. In various embodiments, the scaffold 210 may comprise a hydrogel, an aerogel, a cryogel, an organogel, a nanogel, a conductive hydrogel, or any combination thereof.

Exemplary flow cytometry methods of use and compositions may be used in conjunction with the presently disclosed synthetic biomarker-based standard 200 which are described in International Patent Application WO2022/093925, U.S. Patent Publication No. US2022/0155299A1 and US2024/0168016A1 the disclosures of which are herein incorporated by reference in their entireties for all purposes.

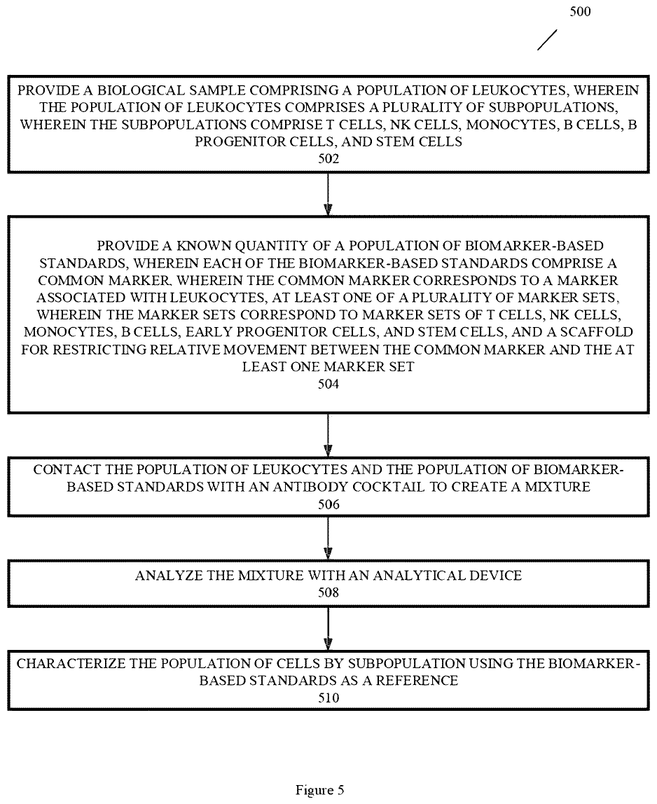

FIG. 5 illustrates a process 500 for characterizing a population of cells into subpopulations using biomarker-based standards, according to various embodiments.

In various embodiments, the method may comprise providing a biological sample comprising a population of leukocytes, wherein the population of leukocytes comprises a plurality of subpopulations, wherein the subpopulations comprise T cells, NK cells, monocytes, B cells, B progenitor cells, and stem cells 502.

In various embodiments, the method may comprise providing a known quantity of a population of biomarker-based standards, wherein the biomarker-based standards comprise a common marker, wherein the common marker corresponds to a marker associated with leukocytes, at least one of a plurality of marker sets, wherein the marker sets correspond to marker sets of T cells, NK cells, monocytes, B cells, early progenitor cells, and stem cells, and a scaffold for restricting relative movement between the common marker and the at least one marker set 504.

In various embodiments, the method may comprise contacting the population of leukocytes and the population of biomarker-based standards with an antibody cocktail to create a mixture 506.

In various embodiments, the method may comprise analyzing the mixture with an analytical device 508. In various embodiments, the analytical device 508 may comprise a flow cytometer 600.

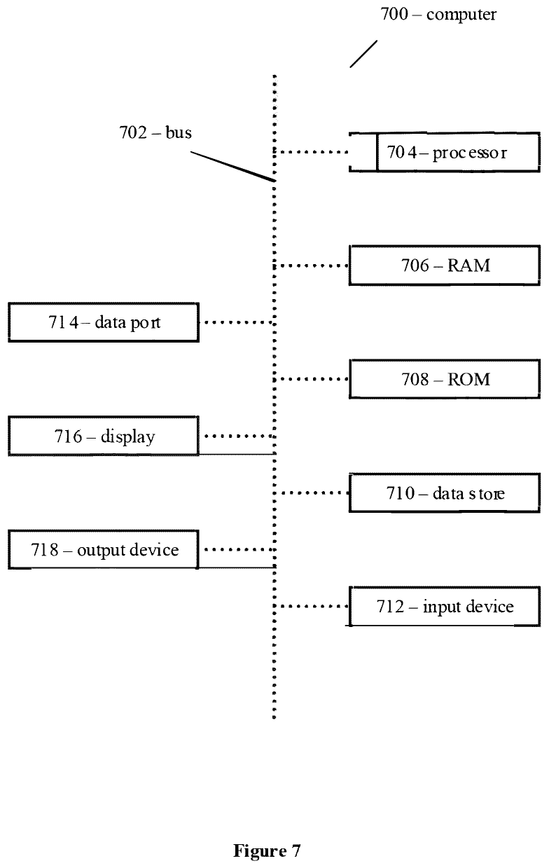

In various embodiments, the method may comprise characterizing the population of cells by subpopulation using the biomarker-based standards as a reference 510. In various embodiments, a computer system 700 such as the one shown in FIG. 7 may store the reference on a data store 710. In various embodiments, the step of characterizing may be carried out by the computer system 700. Processor 704 may process a dataset generated from the analysis by comparing the dataset to the reference.

In various embodiments, the step of characterizing may comprise determining a quantity of the population of leukocytes and quantities for each of the subpopulations.

In various embodiments, the step of characterizing comprises determining relative quantities or percentages of each of the subpopulations and the population of leukocytes.

In various embodiments, the common marker is CD45, wherein CD45 is comprised of populations of CD45hi and CD45dim.

In various embodiments, a marker set of the plurality of marker sets comprises CD10, CD19, CD45dim. In various embodiments, a marker set of the plurality of marker sets comprises CD19, CD34, and CD45dim.

In various embodiments, a marker set of the plurality of marker sets comprises CD3. CD14, and CD45.

In various embodiments, a marker set of the plurality of marker sets comprises CD3, CD45, and CD56.

In various embodiments, a marker set of the plurality of marker sets comprises CD3, CD19, and CD45.

In various embodiments, the plurality of marker sets comprise CD3, CD10, CD14, CD19, CD34, CD45, CD45dim, and CD56.

In various embodiments, at least some of the population of biomarker-based standards further comprise amine binding sites that react to Near IR viability dye. In various embodiments, between about 15-20% of the population of biomarker-based standards comprise amine binding sites. In various embodiments, between about 16.7-100% of the population of biomarker-based standards do not comprise amine binding sites.

In various embodiments, between about 50-60% of the population of biomarker-based standards express CD45hi. In various embodiments, between about 25.2-66.1% of the population of biomarker-based standards express CD45hi. In various embodiments, about 38.2% of the population of biomarker-based standards express CD45hi.

In various embodiments, between about 40-50% of the population of biomarker-based standards express CD45dim. In various embodiments, between about 32.3-74.7% of the population of biomarker-based standards express CD45dim. In various embodiments, about 59.7% of the population of biomarker-based standards express CD45dim.

In various embodiments, between about 50-60% of the population of biomarker-based standards comprise CD10, CD19, and CD45dim. In various embodiments, between about 29.7-66.7% of the population of biomarker-based standards comprise CD10, CD19, and CD45dim. In various embodiments, about 49.2 of the population of biomarker-based standards express CD10, CD19, and CD45dim.

In various embodiments, between about 50-60% of the population of biomarker-based standards comprise CD19, CD34, and CD45dim. In various embodiments, between about 0.3-68.2% of the population of biomarker-based standards comprise CD19, CD34, and CD45dim. In various embodiments, about 45.6% of the population of biomarker-based standards express CD19, CD34, and CD45dim.

In various embodiments, the markers may be embedded within the scaffolds. In various embodiments, the markers may be located on a surface of the scaffolds. In various embodiments, the scaffold comprises a phospholipid bilayer.

In various embodiments, the scaffold comprises a hydrogel, an aerogel, a cryogel, an organogel, a nanogel, a conductive hydrogel, or any combination thereof.

In various embodiments, the method for characterizing a population of cells into subpopulations further comprises an antibody cocktail, wherein the antibody cocktail comprises an antibody for detecting the CD3, an antibody for detecting the CD10, an antibody for detecting the CD14, an antibody for detecting the CD19, an antibody for detecting the CD34, an antibody for detecting the CD45/CD45dim, and an antibody for detecting the CD56.

In various embodiments, the antibody for detecting the CD3 may be conjugated to an APC fluorochrome. In various embodiments, the antibody for detecting the CD10 marker may be conjugated to a FITC fluorochrome. In various embodiments, the antibody for detecting the CD14 marker may be conjugated to a PerCP Cy5.5 fluorochrome. In various embodiments, the antibody for detecting the CD19 may be conjugated to a PE.Cy7 fluorochrome. In various embodiments, the antibody for detecting the CD34 may be conjugated to a BV421 fluorochrome. In various embodiments, the antibody for detecting the CD45/CD45dim marker may be conjugated to a V500 fluorochrome. In various embodiments, the antibody for detecting the CD56 marker may be conjugated to a PE fluorochrome.

In various embodiments, the biological sample comprises an apheresis. In various embodiments, the biological sample comprises a final product.

In various embodiments, the analytical device comprises a flow cytometer. In various embodiments, the flow cytometer comprises a plurality of lasers directed through a flow channel for exciting the fluorochromes. In various embodiments, the flow cytometer comprises at least one detector.

In various embodiments, the step of analyzing further comprises flowing the mixture through the flow cell. In various embodiments, the step of analyzing further comprises interrogating each of the cells in the populations of leukocytes and each of the biomarker-based standards with each of the plurality of lasers. In various embodiments, the step of analyzing further comprises capturing signal intensity data for each marker.

### VII. Exemplary Analytical Systems and Devices

In various embodiments, systems and devices may be used to analyze cells or biomarker-based standards comprising a marker bound to a labeled detection molecule. A variety of commercially available analytical devices may be available including cell counters, polymerase chain reaction (PCR) based technology, and flow cytometry systems and methods.

In various embodiments, flow cytometry technology be used in conjunction with the compositions, kits, and methods described herein. In various embodiments, flow cytometry may enable fast and accurate identification of a cell of a population or subpopulation of cells.

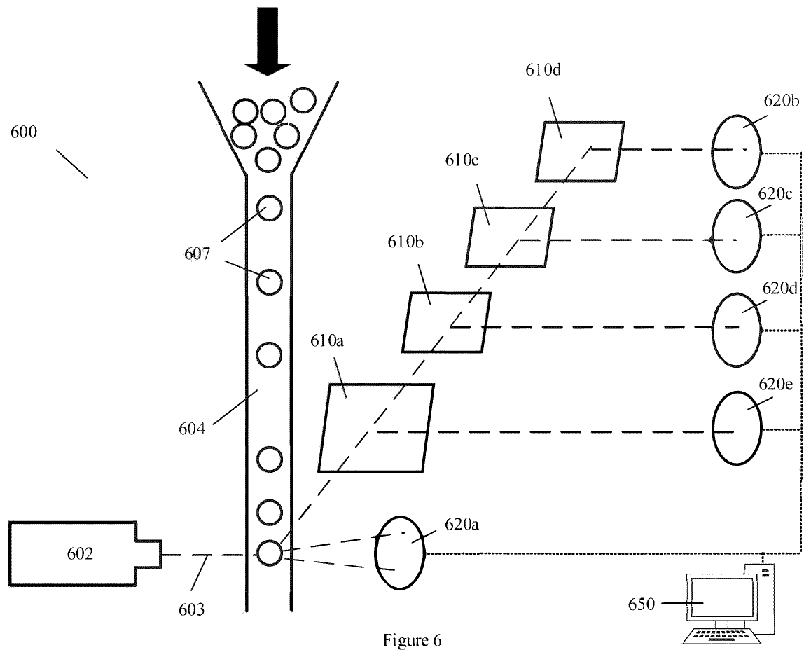

FIG. 6 is a schematic diagram of a flow cytometer 600 for carrying out methods using biomarker-based standards, according to various embodiments. In various embodiments, biomarker-based standards may be particularly suitable for use in flow cytometers 600.

In various embodiments, the flow cytometer 600 may comprise a plurality of lasers 602 for directing a plurality of laser beams 603 through a flow channel 604 for exciting fluorochromes of a mixture undergoing analysis. In various embodiments, the fluorochromes may be associated with labeled detection molecules and the labeled detection molecules may be bound a marker described herein.

In various embodiments, the method for characterizing a population of cells into subpopulations further comprises flowing the mixture through the flow cell 604 of a flow cytometer 600. In various embodiments, particles 607 (e.g., cells, biomarker-based standards, etc.) may pass through the plurality of lasers 602 and a light beam 603 may be scattered.

In various embodiments, embodiments particles may comprise a population of cells from a biological sample. In various embodiments, embodiments particles may comprise a population of biomarker-based standards.

Forward scattered light may be detected by a forward scatter detector 620a. Side scattered light may pass through one or more filters 610a, 610b, 610c, 610d. The side scattered light may then be detected by at least one detector 620b, 620c, 620d, 620e.

In various embodiments, the method for characterizing a population of cells into subpopulations further comprises interrogating each of the cells in the populations of leukocytes and each of the biomarker-based standards with each of the plurality of lasers 602. In various embodiments, each of the plurality of lasers 602 may be selected to detect one or more labeled detection molecules.

In various embodiments, a laser 602 may be selected for detecting an APC fluorochrome. In various embodiments, a laser 602 may be selected for detecting a FITC fluorochrome. In various embodiments, a laser 602 may be selected for detecting a PerCP Cy5.5 fluorochrome. In various embodiments, a laser 602 may be selected for detecting a PE.Cy7 fluorochrome. In various embodiments, a laser 602 may be selected for detecting a BV421 fluorochrome. In various embodiments, a laser 602 may be selected for detecting a V500 fluorochrome. In various embodiments, a laser 602 may be selected for detecting a PE fluorochrome.

In various embodiments, the method for characterizing a population of cells into subpopulations further comprises capturing signal intensity data for each marker. In various embodiments, the signal intensity data for each marker may be detected by one or more detectors 602a, 602b, 602c, 602d, 602e of flow cytometer 600.

In various embodiments, signal intensity data may be communicated electronically to computer system 650 from the one or more detectors 602a, 602b, 602c, 602d, 602e of flow cytometer 600. In various embodiments, computer system 650 may comprise one or more computers and the one or more computers may be networked by any method described herein or elsewhere.

In various embodiments, computer system 650 may be responsible for further processing of a signal intensity data. For example, computer system 650 may be responsible for characterizing the population of cells by subpopulation using the biomarker-based standards as a reference.

In various embodiments, computer system 650 may process signal intensity data into cell population data. Cell population data may comprise, total number of leukocytes, total of each subpopulation, percentage subpopulation total to total leukocyte population, etc. Cell population data may comprise any statically relevant information derivable from knowing an identity for each cell in the population. In various embodiments, computer system 650 may comprise one or more computer systems 700.

FIG. 7 is a schematic diagram of a computer system 700 in accordance with various embodiments. In various embodiments, one or more computer systems 700 may be used independently or in conjunction with one or more additional hardware elements (e.g., analytical systems or devices). One or more computer systems 700 may be used to execute any of the methods described herein. One or more computer systems may be used to process raw data (e.g., mean fluorescence [MFI], amplitude, image, etc.) into a form that may be user friendly, statistically significant, and/or reveal useful information.

The computer system 700, upon which embodiments of the present teachings may be implemented. In various embodiments, a computer system 700 may be integrated into other parts of the system. For example, a controller, a data logger, a cell phone tower, a user device, or almost any other aspect of the system may include an integrated computer system 700. Non-limiting functions of a computer system 700 may include operation of processes (e.g., feedback loops) described herein, processing data received from sensors, providing communication between systems and operators, providing digital storage, etc.

In various embodiments of the present teachings, computer system 700 may include a bus 702 or other communication mechanism for communicating information, and a processor 704 coupled with bus 702 for processing information. In various embodiments, computer system 700 may also include a memory, which can be a random-access memory (RAM) 706 or other dynamic storage device, coupled to bus 702 for determining instructions to be executed by processor 704. Memory also can be used for storing temporary variables or other intermediate information during execution of instructions to be executed by processor 704. In various embodiments, computer system 700 may further include a read only memory (ROM) 708 or other static storage device coupled to bus 702 for storing static information and instructions for processor 704. A data store 710, such as a magnetic disk or optical disk, can be provided and coupled to bus 702 for storing information and instructions.

In some embodiments, computer system 700 can be coupled via bus 702 to a display 716, such as a cathode ray tube (CRT), liquid crystal display (LCD), or light emitting diode display (LED) for displaying information to a computer user. An input device 712, including alphanumeric and other keys, can be coupled to bus 702 for communicating information and command selections to processor 704. Another type of user input device 712 is a cursor control, such as a mouse, a trackball or cursor direction keys for communicating direction information and command selections to processor 704 and for controlling cursor movement on display 716.

In various embodiments, the computer system 700 may include a touchscreen display. The input device 712 typically has two degrees of freedom in two axes, a first axis (i.e., x) and a second axis (i.e., y), that allows the device to specify positions in a plane. However, it should be understood that input devices 712 allowing for 3-dimensional (x, y and z) cursor movement are also contemplated herein.

In various embodiments, computer system 700 can be coupled via bus 702 to one or more data ports 714. In various embodiments, the one or more data ports 714 may enable electronic communication between the components via bus 702 of the computer system 700 and other components of the overall system described herein.

Consistent with certain implementations of the present teachings, results can be provided by computer system 700 in response to processor 704 executing one or more sequences of one or more instructions contained in memory 706. Such instructions can be read into memory 706 from another computer-readable medium or computer-readable storage medium, such as a storage device containing information relating to environmental control (e.g., a feedback algorithm) or an environmental condition monitoring system. Execution of the sequences of instructions contained in memory 706 can cause processor 704 to perform the processes described herein. Alternatively, hard-wired circuitry can be used in place of or in combination with software instructions to implement the present teachings. Thus, implementations of the present teachings are not limited to any specific combination of hardware circuitry and software.

According to various embodiments, computer-readable medium (e.g., data store, data storage, etc.) or computer-readable storage medium may comprise any media that participates in providing instructions to processor 704 for execution. Such a medium can take many forms, including but not limited to, non-volatile media, volatile media, and transmission media. Non-limiting examples of non-volatile media can include optical, solid state, and magnetic disks, such as 708. Examples of volatile media can include, but are not limited to, dynamic memory, such as memory 706. Examples of transmission media can include, but are not limited to, coaxial cables, copper wire, and fiber optics, including the wires that comprise bus 702.

Common forms of computer-readable media include, for example, a floppy disk, a flexible disk, hard disk, magnetic tape, or any other magnetic medium, a CD-ROM, any other optical medium, punch cards, paper tape, any other physical medium with patterns of holes, a RAM, PROM, and EPROM, a FLASH-EPROM, any other memory chip or cartridge, or any other tangible medium from which a computer can read.

In addition to computer readable medium, instructions or data can be provided as signals on transmission media included in a communications apparatus or system to provide sequences of one or more instructions to processor 704 of computer system 700 for execution. For example, a communication apparatus may include a transceiver having signals indicative of instructions and data. The instructions and data are configured to cause one or more processors to implement the functions outlined in the disclosure herein. Representative examples of data communications transmission connections can include, but are not limited to, telephone modem connections, wide area networks (WAN), local area networks (LAN), infrared data connections, NFC connections, etc. In various embodiments, output devices 718 such as printers and displays may be used to present output files generated by the processes described herein.

Output devices 718 may be used to communicate real time environmental conditions or a history of environmental conditions. Output device 718 may be used to indicate the condition or one or more metrics associated with the condition of an apheresis material within the system described herein.

In various embodiments, data may be generated received by computer 700 from flow cytometer 600. In various embodiments, a computer system may be used to characterize a population of cells by subpopulation using the biomarker-based standards as a reference.

In various embodiments, data port 714 may receive MFI data from flow cytometer 600. Data port 714 may transmit MFI data to any component electronically connected by bus 702.

In various embodiments, data from the flow cytometer 600 may be stored on the datastore 710 for long term storage. Additionally, data that has been processed by processor 704 may be stored in data store 710.

In various embodiments, processed data may comprise cell population and/or cell subpopulation statistical information (e.g., quantities of each subpopulation relative to the whole).

In various embodiments, processed data may be manipulated using input device 712. In various embodiments, processed data may be viewed on display 716. In various embodiments, processed data may be sent to output device 718 for printing and/or off-system electronic storage.

### VIII. Examples

Our research efforts focused on identification of useful product (e.g., T cells) and impurities of interest (e.g., leukocytes and leukocyte subpopulations), development of marker sets and labels, assay methods, new standards (e.g. synthetic biomarker-based standard) etc. The examples below describe design and optimization efforts for the biomarker-based standard described herein.

In various embodiments, a cell population may comprise leukocytes and subpopulations of interest may include monocytes, T cells, NK cells, B cells, early progenitor cells, and/or stem cells. The biomarker-based standards described herein are optimized to characterize these cell populations which has never been effectively done until now.

Table 8 lists fluorochrome/antibody pairs that were used to generate data in the examples and their manufacturers. Buffers, live/dead stain, and scaffolds that were used are also listed in Table 8.

TABLE 8
List of Reagents Used

| Reagent | Manufacturer |
|---|---|
| APC anti-human CD3 | Biolegend |
| FITC anti-human CD10 | Biolegend |
| PerCP-Cy5.5 anti-human CD14 | BD Biosciences |
| PE-Cy7 anti-human CD19 | BD Biosciences |
| BV421 anti-human CD34 | Biolegend |
| PE anti-human CD56 | BD Biosciences |
| BV421 mouse IgG2a | Biolegend |
| PE mouse IgG2b | Biolegend |
| FITC mouse IgG1 | Biolegend |
| V500 anti-human CD45 | BD Biosciences |
| LIVE/DEAD Fixable Near-IR Dead Cell Stain | Thermo Fisher |
| Kit | |
| Cytometer Set-up and Tracking (CS&T) beads | BD Biosciences |
| UltraComp eBeads Compensation Beads | Thermo Fisher |
| | Scientific |
| Cell Stain Buffer (BSA) | BD Biosciences |

### Example: Product Specification

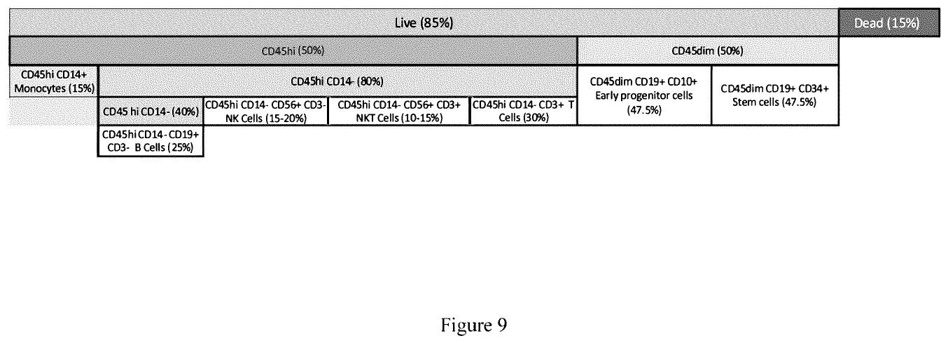

FIG. 9 illustrates an exemplary leukocyte population, according to various embodiments. In various embodiments, FIG. 9 may act as a specification or product specification to be used in the methods described herein. In various embodiments, FIG. 9 may serve as the reference appearing in the methods described herein. In various embodiments, other specifications may be determined that deviate and or comprise ranges relative to the specification described in FIG. 9 (e.g., see range values in Definitions section).

In various embodiments, specifications may be based product needs such as percentage of T cells versus impurity percentage. In various embodiments, certain impurities may have a disproportionately negative impact on a final product. In various embodiments, certain impurities may have a substantially neutral impact, with the exception of crowing out useable product, thereby, decreasing product concentration. In various embodiments, specifications may allow for flexibility. For example, in various embodiments, if one cell type is out of specification another cell type having a decreased quantity and/or relative quantity may positively impact whether a product passes an inspection.

In various embodiments, a product specification or specification may comprise about 6.4% monocytes, about 3.4% B cells, about 5.1% NK cells, about 5.1% NKT cells, about 10.2% T cells, about 20.2% early progenitor cells, about 20.2% stem cells, and between about 15% to about 30% dead or other cell types. In various embodiments, how closely a product matches a product specification (i.e. a comparison) may determine whether a product gets used (i.e. receives a pass determination for a product quality designation). In various embodiments, the specification may allow for a deviation of the specification. of 0.1%, 0.2%, 0.3%, 0.4%, 0.5%, 0.6%, 0.7%, 0.8%, 0.9%, 1.0%, 1.1%, 1.2%, 1.3%, 1.4%, 1.5%, 1.6%, 1.7%, 1.8%, 1.9%, or 2.0%.

In various embodiments, a specification may allow for an increased relative value of T cells and 30% may be a baseline. In various embodiments, a specification may allow for a decreased relative value of dead cells to compensate for other negative values (e.g., one or more out of spec cell counts). In various embodiments, the above values in this paragraph represent maximum values with the exception of T cells.

In various aspects, a single targeting molecule (e.g., an antibody) may be used to target a single target (e.g., an antigen). In various embodiments, a dye may be used to identify live cells versus dead cells. Referring to FIG. 9, a product specification may comprise about 85% live and 15% dead cells.

In various embodiments, a cell population may be categorized by live or dead. Such a determination may be effectuated by a commercially available dye. For example, Thermo Fisher Scientific, Inc.™ offers Invitrogen™ Live/Dead Fixable Near IR (876)™ for sale in their current product catalog. A variety of excitation wavelengths may be available. In various embodiments, a live/dead dye may emit light at a wavelength of 785 nm. In various embodiments, an excitation laser of 785 nm may be used with an analytical device. In various embodiments, a live/dead dye may emit light at a wavelength of 808 nm. In various embodiments, an excitation laser of 808 nm was used with an analytical device.

Referring back to FIG. 9, interrogating for and detection of CD45dim, CD19, and CD10 may allow for determination of presence and quantity of a subpopulation, early progenitor cells. Further, in various embodiments, interrogating for CD45, CD19, and CD10 may allow determination of a relative quantity compared to a population of cells (e.g., a percentage of early progenitor cells compared to a leukocyte population).

Interrogating for CD19 and CD34 may allow for determination of presence and quantity of early progenitor cells.

In various embodiments, the term leukoctye refers to many different kinds of white blood cells. Non-limiting examples of leukocytes include T cells, NK cells, monocytes, B cells, Early B progenitor cells, and stem cells. As such, in various embodiments, CD45 may be present and detectable on leukocytes. In various embodiments, CD45 may be present and detectable on T cells, NK cells, monocytes, B cells, Early B progenitor cells, and stem cells.

In various embodiments, CD45 may be used as a lymphocyte marker used to identify white blood cells. In various embodiments, an analytical tool used to evaluate the presence and/or a relative quantity of CD45 may include a flow cytometer. Flow cytometers have the ability to detect and quantify intensity ranges between several orders of magnitude. As such, CD45 comprises CD45hi (or CD45) and CD45dim.

In various embodiments, a CD45hi marker may identify mature lymphocytes and a CD45dim. In various embodiments, a CD45dim marker may identify stem cells and early progenitor cells.

### Example 2: Determining Biomarker-Based Standard Marker Concentrations

To design an optimized synthetic positive control, we first determined the leukocyte markers that enable identification of sub-populations. Additionally, we determined how much of each marker is present in the overall populations and subpopulations to guide testing for our biomarker-based standards.

Live leukocytes were collected from a variety of donors ranging from healthy to diseased. The leukocytes were labeled and analyzed in accordance with the flow cytometry methods described in International Patent Application WO2022/093925, U.S. Patent Publication No. US2022/0155299A1 and US2024/0168016A1 the disclosures of which previously were and are herein incorporated by reference in their entireties for all purposes.

### A. Clinical Sample—CD45hi

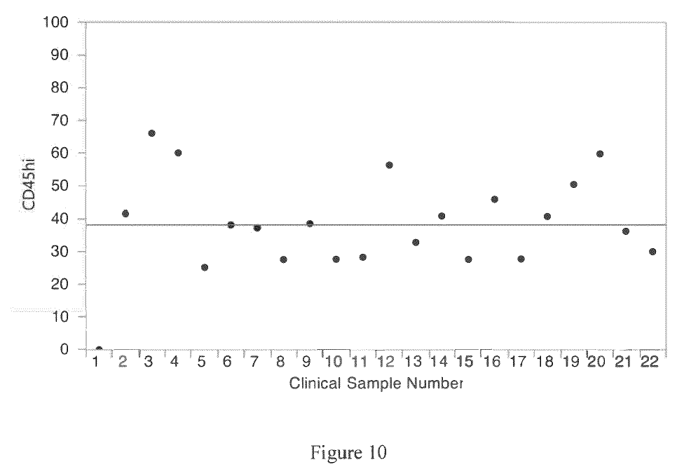

CD45hi marker can be found on monocytes, NK cells, T cells, and B cells. FIG. 10 is experimental flow cytometry data from diseased biological samples showing the percentage of leukocyte populations expressing CD45hi marker. Biological samples originating from donors 1-22 were used. The donors range from various disease states for broad representation across all populations. The y-axis includes percentage of the leukocyte population expressing CD45hi for a given clinical sample. Clinical sample or donor numbers are shown on the x-axis. Table 9 includes the same data as FIG. 10 using numerical values. On average, 38.2 percent of leukocytes across all donors express CD45hi. The lowest expression level of CD45hi from a donor was 0 percent. The lowest non-zero expression level of CD45hi from a donor was 0.1 percent. The highest expression level of CD45hi from a donor was 66.1.

TABLE 9
Leukocyte Subpopulation Analysis for CD45hi Marker

| | CD45hi % |
|---|---|
| Donor Identification | Positive Cells |
| Donor 1 | 0 |
| Donor 2 | 41.6 |
| Donor 3 | 66.1 |
| Donor 4 | 60.1 |
| Donor 5 | 25.2 |
| Donor 6 | 38.2 |
| Donor 7 | 37.3 |
| Donor 8 | 27.6 |
| Donor 9 | 38.6 |
| Donor 10 | 27.7 |
| Donor 11 | 28.3 |
| Donor 12 | 56.4 |
| Donor 13 | 32.8 |
| Donor 14 | 40.9 |
| Donor 15 | 27.6 |
| Donor 16 | 45.9 |
| Donor 17 | 27.8 |
| Donor 18 | 40.8 |
| Donor 19 | 50.4 |
| Donor 20 | 59.8 |
| Donor 21 | 36.2 |
| Donor 22 | 30.0 |
| Average | 38.2 |
| Highest | 66.1 |
| Lowest | 0 |
| Lowest non-0 | 25.2 |

### B. Clinical Sample—CD45dim

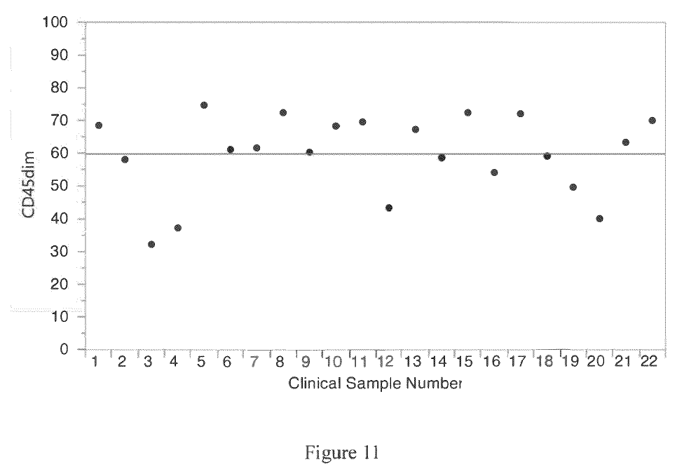

CD45dim marker can be found on early progenitor cells and stem cells. FIG. 11 is experimental flow cytometry data from diseased biological samples showing the percentage of leukocyte populations expressing CD45dim marker. Biological samples originating from donors 1-22 were used. The donors range from various disease states for broad representation across all populations. The y-axis includes percentage of the leukocyte population expressing CD45dim for a given clinical sample. Clinical sample or donor numbers are shown on the x-axis. Table 10 includes the same data as FIG. 11 using numerical values. On average, 59.7 percent of leukocytes across all donors express CD45dim. The lowest expression level of CD45dim from a donor was 32.3 percent. The highest expression level of CD45dim from a donor was 74.7.

TABLE 10
Leukocyte Subpopulation Analysis for CD45dim Marker

| | CD45dim % |
|---|---|
| Donor Identification | Positive Cells |
| Donor 1 | 68.5 |
| Donor 2 | 58.1 |
| Donor 3 | 32.3 |
| Donor 4 | 37.3 |
| Donor 5 | 74.7 |
| Donor 6 | 61.1 |
| Donor 7 | 61.6 |
| Donor 8 | 72.4 |
| Donor 9 | 60.3 |
| Donor 10 | 68.3 |
| Donor 11 | 69.6 |
| Donor 12 | 43.3 |
| Donor 13 | 67.3 |
| Donor 14 | 58.6 |
| Donor 15 | 72.4 |
| Donor 16 | 54.1 |
| Donor 17 | 72.1 |
| Donor 18 | 59.1 |
| Donor 19 | 49.6 |
| Donor 20 | 40.1 |
| Donor 21 | 63.3 |
| Donor 22 | 70.0 |
| Average | 59.7 |
| Highest | 74.7 |
| Lowest | 32.3 |

### C. Clinical Sample—CD45hi/CD14+

The combination of CD45hi and CD14+ marker can be found on monocytes. Table 11 is experimental flow cytometry data from diseased biological samples showing the percentage of leukocyte populations expressing CD45hi and CD14+ marker. Biological samples originating from donors 1-22 were used. The donors range from various disease states for broad representation across all populations. On average, 3.4 percent of leukocytes across all donors express CD45hi and CD14+. The lowest expression level of CD45hi and CD14+ from a donor was 0 percent. The lowest non-zero expression level of CD45hi and CD14+ from a donor was 0.1 percent. The highest expression level of CD45hi and CD14+ from a donor was 15.6.

TABLE 11
Leukocyte Subpopulation Analysis for CD45hi / CD14+ Markers

| | CD45hi / CD14+ % |
|---|---|
| Donor Identification | Positive Cells |
| Donor 1 | 15.6 |
| Donor 2 | 0.1 |
| Donor 3 | 3.3 |
| Donor 4 | 0.0 |
| Donor 5 | 0.0 |
| Donor 6 | 7.5 |
| Donor 7 | 0.9 |
| Donor 8 | 0.4 |
| Donor 9 | 9.1 |
| Donor 10 | 0.0 |
| Donor 11 | 0.3 |
| Donor 12 | 0.0 |
| Donor 13 | 11.7 |
| Donor 14 | 3.6 |
| Donor 15 | 0.0 |
| Donor 16 | 0.1 |
| Donor 17 | 0.2 |
| Donor 18 | 6.3 |
| Donor 19 | 0.1 |
| Donor 20 | 2.6 |
| Donor 21 | 9.2 |
| Donor 22 | 4.9 |
| Average | 3.4 |
| Highest | 15.6 |
| Lowest | 0.1 |

### D. Clinical Sample—CD45hi/CD14−/CD56−/CD3+

The combination of CD45hi and CD3+ markers lacking CD14 and CD56 can be found on T cells. Table 12 is experimental flow cytometry data from diseased biological samples showing the percentage of leukocyte populations expressing CD45hi and CD3+ marker while lacking CD14 and CD56 markers. Biological samples originating from donors 1-22 were used. The donors range from various disease states for broad representation across all populations. On average, 23.8 percent of leukocytes across all donors expressed CD45hi and CD3+ markers while not expressing CD14 and CD56. The lowest expression level of CD45hi and CD3+ markers in the absence of CD14 and CD56 expression from a donor was 5.7 percent. The highest expression level of CD45hi and CD3+ markers without expression of CD14 and CD56 from a donor was 50.4.

TABLE 12
Leukocyte Subpopulation Analysis for CD45hi / CD3+
Markers (CD14− / CD56−)

| | CD45hi / CD3+ Markers |
|---|---|
| Donor Identification | (CD14− / CD56−) |
| Donor 1 | 5.7 |
| Donor 2 | 33.9 |
| Donor 3 | 43.7 |
| Donor 4 | 39.3 |
| Donor 5 | 11.6 |
| Donor 6 | 18.7 |
| Donor 7 | 28.5 |
| Donor 8 | 21.3 |
| Donor 9 | 19.9 |
| Donor 10 | 10.6 |
| Donor 11 | 10.5 |
| Donor 12 | 50.4 |
| Donor 13 | 9.0 |
| Donor 14 | 25.9 |
| Donor 15 | 18.6 |
| Donor 16 | 28.2 |
| Donor 17 | 16.8 |
| Donor 18 | 24.7 |
| Donor 19 | 40.0 |
| Donor 20 | 38.5 |
| Donor 21 | 13.7 |
| Donor 22 | 13.7 |
| Average | 23.8 |
| Highest | 50.4 |
| Lowest | 5.7 |

### E. Clinical Sample—CD45hi/CD14−/CD19+/CD3− (CD56−)

The combination of CD45hi and 19+ markers while lacking CD14 and CD3 can be found on B cells. Table 13 is experimental flow cytometry data from diseased biological samples showing the percentage of leukocyte populations expressing CD45hi and CD19+ marker while lacking expression of CD14 and CD3. Biological samples originating from donors 1-22 were used. The donors range from various disease states for broad representation across all populations. On average, 3.1 percent of leukocytes across all donors expressed CD45hi and CD19+ markers while not expressing CD14 and CD3. The lowest expression level of CD45hi and CD19+ markers in the absence of CD14 and CD3 expression from a donor was 0.1 percent. The highest expression level of CD45hi and CD19+ markers without expression of CD14 and CD3 from a donor was 14.5.

TABLE 13
Leukocyte Subpopulation Analysis for CD45hi / CD19+
Markers (CD14− / CD3−)

| | CD45hi / CD19+ Markers |
|---|---|
| Donor Identification | (CD14− / CD3−) |
| Donor 1 | 0.7 |
| Donor 2 | INVALID |
| Donor 3 | 1.1 |
| Donor 4 | INVALID |
| Donor 5 | 0.6 |
| Donor 6 | 0.9 |
| Donor 7 | 0.1 |
| Donor 8 | 1.6 |
| Donor 9 | 3.3 |
| Donor 10 | INVALID |
| Donor 11 | 14.5 |
| Donor 12 | 0.2 |
| Donor 13 | 1.1 |
| Donor 14 | 0.8 |
| Donor 15 | 2.5 |
| Donor 16 | 12.3 |
| Donor 17 | INVALID |
| Donor 18 | 0.3 |
| Donor 19 | 5.6 |
| Donor 20 | 2.6 |
| Donor 21 | 1.9 |
| Donor 22 | 5.8 |
| Average | 3.1 |
| Highest | 14.5 |
| Lowest | 0.1 |

### F. Clinical Sample—CD45hi/CD14−/CD56+CD3− (CD19−)

The combination of CD45hi and CD56+ markers lacking CD14 and CD3 can be found on NK cells. Table 14 is experimental flow cytometry data from diseased biological samples showing the percentage of leukocyte populations expressing CD45hi and CD56+ marker while lacking expression of CD14 and CD3. Biological samples originating from donors 1-22 were used. The donors range from various disease states for broad representation across all populations. On average, 5.7 percent of leukocytes across all donors expressed CD45hi and CD56+ markers while not expressing CD14 and CD3. The lowest expression level of CD45hi and CD56+ markers in the absence of CD14 and CD3 expression from a donor was 1.1 percent. The highest expression level of CD45hi and CD56+ markers without expression of CD14 and CD3 from a donor was 14.6.

TABLE 14
Leukocyte Subpopulation Analysis for CD45hi / CD56+
Markers (CD14− / CD3− / CD19−)

| | CD45hi / CD56+ Markers |
|---|---|
| Donor Identification | (CD14−/ CD3−) |
| Donor 1 | 5.6 |
| Donor 2 | 5.3 |
| Donor 3 | 9.2 |
| Donor 4 | 4.3 |
| Donor 5 | 9.9 |
| Donor 6 | 3.9 |
| Donor 7 | 5.1 |
| Donor 8 | 1.1 |
| Donor 9 | 3.0 |
| Donor 10 | 14.6 |
| Donor 11 | 2.2 |
| Donor 12 | 4.4 |
| Donor 13 | 4.9 |
| Donor 14 | 5.7 |
| Donor 15 | 1.4 |
| Donor 16 | 1.7 |
| Donor 17 | 3.9 |
| Donor 18 | 1.6 |
| Donor 19 | 2.3 |
| Donor 20 | 10.9 |
| Donor 21 | 9.3 |
| Donor 22 | 3.7 |
| Average | 5.7 |
| Highest | 14.6 |
| Lowest | 1.1 |

### G. Clinical Sample—CD45hi/CD54−/CD56+/CD3+(CD19−)

The combination of CD45hi, CD56+, and CD3+ markers while lacking CD14 can be found on NKT cells. Table 15 is experimental flow cytometry data from diseased biological samples showing the percentage of leukocyte populations expressing CD45hi, CD56+, and CD3+ marker while lacking expression of CD14. Biological samples originating from donors 1-22 were used. The donors range from various disease states for broad representation across all populations. On average, 3.2 percent of leukocytes across all donors expressed CD45hi, CD56+, and CD3+ markers while not expressing CD14. The lowest expression level of CD45hi, CD56+, and CD3+ markers in the absence of CD14 expression from a donor was 0.2 percent. The highest expression level of CD45hi, CD56+, and CD3+ markers without expression of CD14 from a donor was 16.4.

TABLE 15
Leukocyte Subpopulation Analysis for CD45hi / CD14− / CD56+ /
CD3+ Markers (CD19−)

| | CD45hi / CD56+ / CD3+ |
|---|---|
| Donor Identification | Markers (CD3−) |
| Donor 1 | 0.2 |
| Donor 2 | 2.2 |
| Donor 3 | 3.9 |
| Donor 4 | 16.4 |
| Donor 5 | 3.0 |
| Donor 6 | 2.0 |
| Donor 7 | 1.3 |
| Donor 8 | 2.0 |
| Donor 9 | 1.1 |
| Donor 10 | 1.9 |
| Donor 11 | 0.6 |
| Donor 12 | 1.1 |
| Donor 13 | 2.3 |
| Donor 14 | 4.3 |
| Donor 15 | 4.9 |
| Donor 16 | 3.3 |
| Donor 17 | 6.6 |
| Donor 18 | 4.8 |
| Donor 19 | 1.4 |
| Donor 20 | 4.7 |
| Donor 21 | 0.6 |
| Donor 22 | 0.8 |
| Average | 3.2 |
| Highest | 16.4 |
| Lowest | 0.2 |

### H. Clinical Sample—CD4dim/CD19+/CD10+

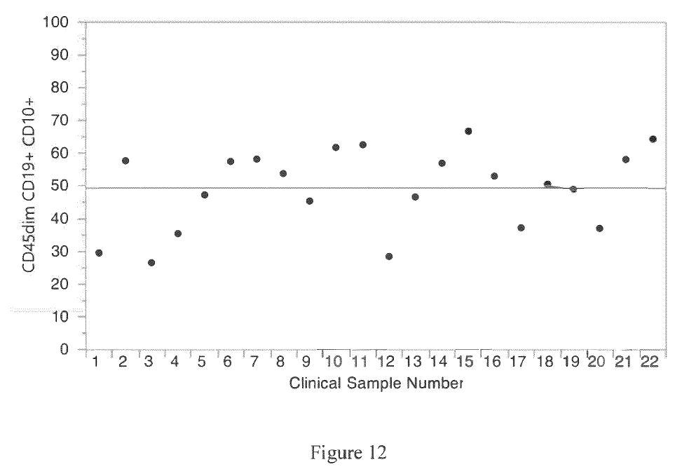

The combination of CD45dim, CD19+, and CD10+ markers can be found on early progenitor cells. FIG. 12 is experimental flow cytometry data from diseased biological samples showing the percentage of leukocyte populations expressing CD45dim, CD19+, and CD10+. Biological samples originating from donors 1-22 were used. The donors range from various disease states for broad representation across all populations. The y-axis includes percentage of the leukocyte population expressing CD45dim, CD19+, and CD10+ markers for a given clinical sample. Clinical sample or donor numbers are shown on the x-axis. Table 16 includes the same data as FIG. 12 using numerical values. On average, 49.2 percent of leukocytes across all donors expressed CD45dim, CD19+, and CD10+ markers. The lowest expression level of CD45dim, CD19+, and CD0+ markers expression from a donor was 29.7 percent. The highest expression level of CD45dim, CD19+, and CD10+ markers from a donor was 66.7.

TABLE 16
Leukocyte Subpopulation Analysis for CD45dim / CD19+ / CD10+

| Donor Identification | CD45dim / CD19+ / CD10+ |
|---|---|
| Donor 1 | 29.7 |
| Donor 2 | 57.7 |
| Donor 3 | 26.6 |
| Donor 4 | 35.5 |
| Donor 5 | 47.2 |
| Donor 6 | 57.5 |
| Donor 7 | 58.2 |
| Donor 8 | 53.8 |
| Donor 9 | 45.4 |
| Donor 10 | 61.7 |
| Donor 11 | 62.6 |
| Donor 12 | 28.5 |
| Donor 13 | 46.6 |
| Donor 14 | 56.9 |
| Donor 15 | 66.7 |
| Donor 16 | 53.0 |
| Donor 17 | 37.3 |
| Donor 18 | 50.5 |
| Donor 19 | 49.0 |
| Donor 20 | 37.1 |
| Donor 21 | 58.0 |
| Donor 22 | 64.3 |
| Average | 49.2 |
| Highest | 66.7 |
| Lowest | 29.7 |

### I. Clinical Sample—CD45dim/CD19+/CD34+

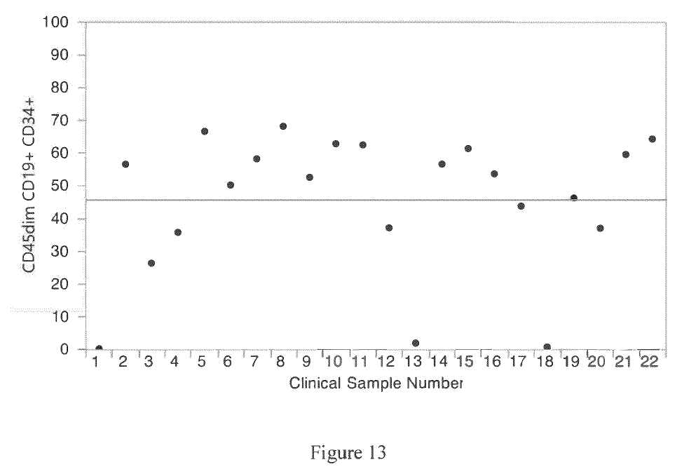

The combination of CD45dim CD19+, and CD34+ markers can be found on stem cells. FIG. 13 is experimental flow cytometry data from diseased biological samples showing the percentage of leukocyte populations expressing CD45dim, CD19+, and CD34+. Biological samples originating from donors 1-22 were used. The donors range from various disease states for broad representation across all populations. The y-axis includes percentage of the leukocyte population expressing CD45dim, CD19+, and CD34+ markers for a given clinical sample. Clinical sample or donor numbers are shown on the x-axis. Table 17 includes the same data as FIG. 13 using numerical values. On average, 45.6 percent of leukocytes across all donors expressed CD45dim, CD19+, and CD34+ markers. The lowest expression level of CD45dim, CD19+, and CD10+ markers expression from a donor was 0.3 percent. The highest expression level of CD45dim, CD19+, and CD34+ markers from a donor was 68.2.

TABLE 17
Leukocyte Subpopulation Analysis for CD45dim / CD19+ / CD34+

| Donor Identification | CD45dim / CD19+ / CD34+ |
|---|---|
| Donor 1 | 0.3 |
| Donor 2 | 56.6 |
| Donor 3 | 26.5 |
| Donor 4 | 35.9 |
| Donor 5 | 66.7 |
| Donor 6 | 50.2 |
| Donor 7 | 58.3 |
| Donor 8 | 68.2 |
| Donor 9 | 52.6 |
| Donor 10 | 62.9 |
| Donor 11 | 62.5 |
| Donor 12 | 37.3 |
| Donor 13 | 2.0 |
| Donor 14 | 56.7 |
| Donor 15 | 61.4 |
| Donor 16 | 53.7 |
| Donor 17 | 43.7 |
| Donor 18 | 0.8 |
| Donor 19 | 46.3 |
| Donor 20 | 37.2 |
| Donor 21 | 59.6 |
| Donor 22 | 64.3 |
| Average | 45.6 |
| Highest | 68.2 |
| Lowest | 0.3 |

### J. Clinical Sample—CD45− Near IR+

The combination of CD45− Near IR+ markers indicate the cells are dead. Table 18 is experimental flow cytometry data from diseased biological samples showing the percentage of live leukocytes (e.g., the population not bound to Near IR+ stain). Biological samples originating from donors 1-22 were used. The donors range from various disease states for broad representation across all populations. On average, 94.5 percent of leukocytes across all donors were living. On average, 5.5 percent of leukocytes across all donors were dead as indicated by bound Near IR+ stain. The highest population of living cells was 100 percent. The lowest population of living cells was 16.7 percent.

TABLE 18
Leukocyte Subpopulation Analysis for CD45− Near IR+

| Donor Identification | Live Leukocytes |
|---|---|
| Donor 1 | 99.5 |
| Donor 2 | 100.0 |
| Donor 3 | 96.2 |
| Donor 4 | 94.6 |
| Donor 5 | 99.8 |
| Donor 6 | 100.0 |
| Donor 7 | 99.9 |
| Donor 8 | 16.7 |
| Donor 9 | 99.7 |
| Donor 10 | 100.0 |
| Donor 11 | 99.3 |
| Donor 12 | 100.0 |
| Donor 13 | 100.0 |
| Donor 14 | 100.0 |
| Donor 15 | 100.0 |
| Donor 16 | 96.9 |
| Donor 17 | 91.1 |
| Donor 18 | 98.3 |
| Donor 19 | 96.3 |
| Donor 20 | 92.7 |
| Donor 21 | 99.3 |
| Donor 22 | 100.0 |
| Average | 94.5 |
| Highest | 100.0 |
| Lowest | 16.7 |

### K. Healthy Donor Sample—CD45hi

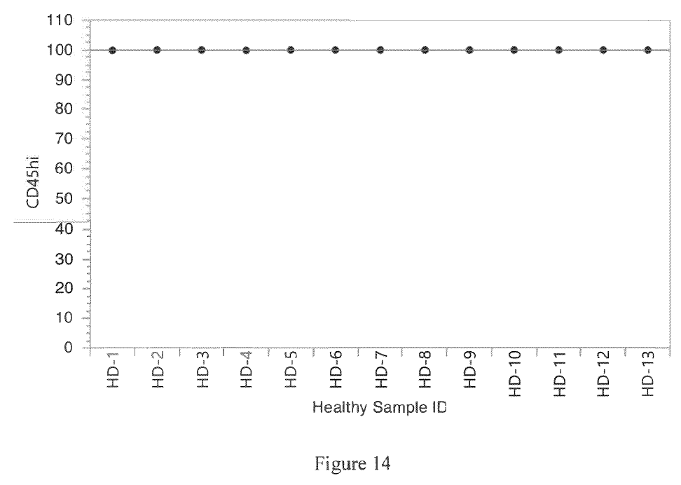

CD45hi marker can be found on monocytes, NK cells, T cells, and B cells. FIG. 14 is experimental flow cytometry data from healthy biological samples showing the percentage of leukocyte populations expressing CD45hi marker. Biological samples originating from donors 1-13 were used. The donors range from various healthy states for broad representation across all populations. The y-axis includes percentage of the leukocyte population expressing CD45hi for a given healthy sample. Healthy Sample IDs shown on the x-axis.

### L. Healthy Donor Sample—CD45dim

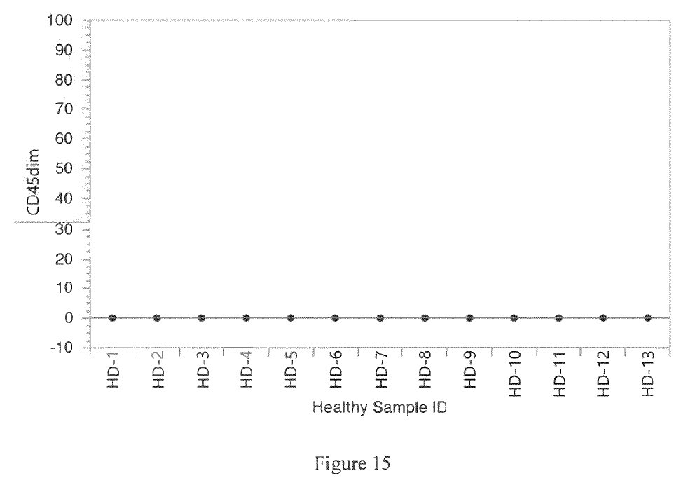

CD45dim marker can be found on early progenitor cells and stem cells. FIG. 15 is flow cytometry data from healthy biological samples showing the percentage of leukocyte populations expressing CD45dim marker. Biological samples originating from donors 1-13 were used. The donors range from various healthy states for broad representation across all populations. The y-axis includes percentage of the leukocyte population expressing CD45dim for a given healthy sample. Healthy Sample IDs shown on the x-axis.

M. Healthy Donor Sample—CD45hi NK cells

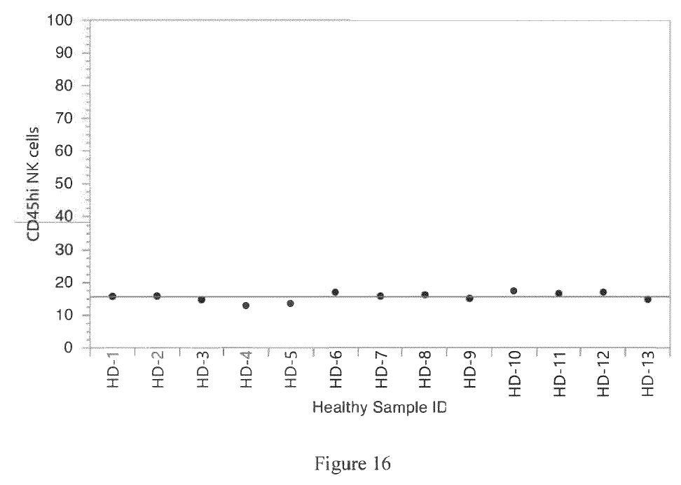

The combination of CD45hi and CD56+ markers while lacking CD14 and CD3 can be found on NK cells. FIG. 16 is experimental flow cytometry data from healthy biological samples showing the percentage of leukocyte populations expressing CD45hi and CD56+ markers and lacking CD14 and CD3. Biological samples originating from donors 1-13 were used. The donors range from various healthy states for broad representation across all populations. The y-axis includes percentage of the leukocyte population expressing CD45hi and CD56+ markers for a given healthy sample. Healthy Sample IDs shown on the x-axis.

### N. Healthy Donor Sample—CD45Hi Cells

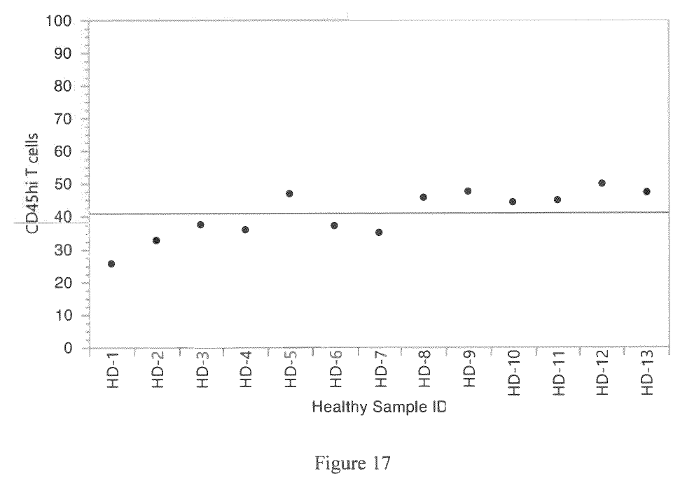

The combination of CD45hi and CD3+ markers lacking CD14 and CD56 can be found on T cells. FIG. 17 is flow cytometry data from healthy biological samples showing the percentage of leukocyte populations expressing CD45hi and CD3+ markers lacking CD14 and CD56. Biological samples originating from donors 1-13 were used. The donors range from various healthy states for broad representation across all populations. The y-axis includes percentage of the leukocyte population expressing CD45hi and CD3+ markers lacking CD14 and CD56 for a given healthy sample. Healthy Sample IDs shown on the x-axis.

### O. Healthy Donor Sample—CD45Hi B Cells

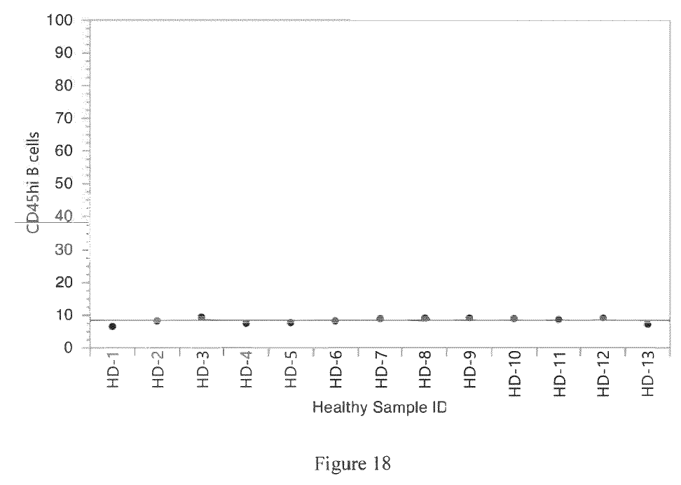

The combination of CD45hi and 19+ markers while lacking CD14 and CD3 can be found on B cells. FIG. 18 is experimental flow cytometry data from healthy biological samples showing the percentage of leukocyte populations expressing CD45hi and 19+ markers while lacking CD14 and CD3. Biological samples originating from donors 1-13 were used. The donors range from various healthy states for broad representation across all populations. The y-axis includes percentage of the leukocyte population expressing CD45hi and 19+ markers while lacking CD14 and CD3 for a given healthy sample. Healthy Sample IDs shown on the x-axis.

### P. Healthy Donor Sample—CD45dim, CD19+, and CD34+

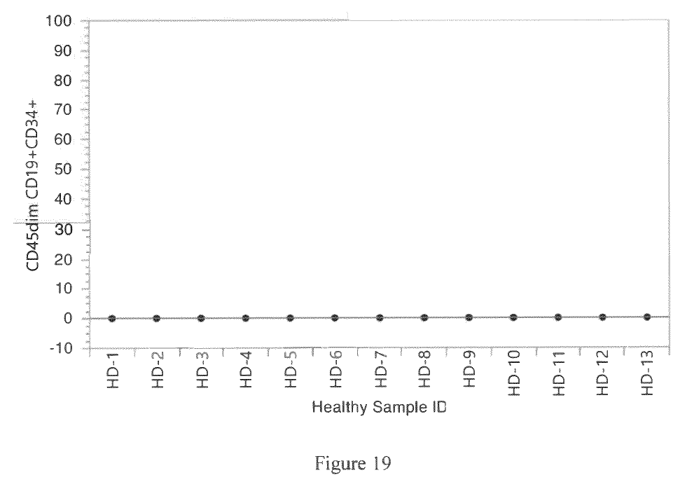

The combination of CD45dim, CD19+, and CD34+ markers can be found on stem cells. FIG. 19 is flow cytometry data from healthy biological samples showing the percentage of leukocyte populations expressing CD45dim, CD19+, and CD34+. Biological samples originating from donors 1-13 were used. The donors range from various healthy states for broad representation across all populations. The y-axis includes percentage of the leukocyte population expressing CD45dim, CD19+, and CD34+ for a given healthy sample. Healthy Sample IDs shown on the x-axis.

### Q. Healthy Donor Sample—CD45Hi B Cells

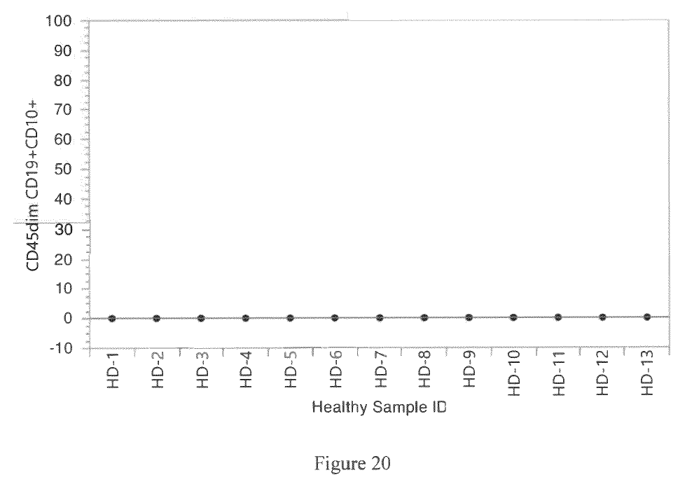

The combination of CD45dim, CD19+, and CD10+ markers can be found on early progenitor cells. FIG. 20 is experimental flow cytometry data from healthy biological samples showing the percentage of leukocyte populations expressing CD45dim, CD19+, and CD10+. Biological samples originating from donors 1-13 were used. The donors range from various healthy states for broad representation across all populations. The y-axis includes percentage of the leukocyte population expressing CD45dim, CD19+, and CD10+ for a given healthy sample. Healthy Sample IDs shown on the x-axis.

### R. Conclusions

Previously existing standards in the art only account for healthy individuals. Previously existing standards do not account for diseased individuals. Example 3 focuses on meeting these unmet needs in the field and solving various other problems.

### Example 3: Biomarker-Based Standard Design

As described in the Background section, prior to development of the standards described herein, existing standards lacked all the necessary markers for characterizing a population of leukocytes. Specifically, existing standards are unable to detect early progenitor cells and stem cells. We needed to fill such a significant unmet need to ensure quality final products consistently meeting or exceeding specification.

TABLE 19
Compiled Marker Expression Data from Example 1

| Marker | Subpopulation | Range | Average |
|---|---|---|---|
| CD45hi | CD45hi | 25.2-66.1 | 38.2 |
| CD45dim | CD45dim | 32.3-74.7 | 59.7 |
| CD14 | CD45hi / CD14+ | 0.1-15.6 | 3.4 |
| CD3 | CD45hi / CD14− / CD3+ | 5.7-50.4 | 23.8 |
| CD19 | CD45hi / CD14− / CD19+ | 0.1-14.5 | 3.1 |
| CD56 | CD45hi / CD14− / CD56+ | 1.1-14.6 | 5.7 |
| CD10 | CD45dim / CD19+ / CD10+ | 29.7-66.7 | 49.2 |
| CD34 | CD45dim / CD19+ / CD34+ | 0.3-68.2 | 45.6 |
| LIVE/DEAD | CD45− Near IR+ | 16.7-100 | 94.5 |
| NEAR-IR | | | |

Using the experimental results from Example 1, the embodiments in tis example section were determined. Specifically, marker sets were developed for representing each of the cell type subpopulations based on the compiled results of Table 19. We investigated a variety of scaffolds for associating markers into marker sets that are spatially restricted to one another. Scaffolds and/or ingredients thereof are commercially available from Thermo Fisher Scientific, Inc.™, BioLengend, Inc.™, BD Biosciences, Inc.™, Slingshot Biosciences, Inc.™ and several others. The examples described herein use commercially available scaffolds that can be supplied from any of the suppliers referenced herein.

In various embodiments, a bead surface may be coated with avidin, streptavidin, and or biotin. In various embodiments, one or more of the markers described herein may be covalently bonded to avidin, streptavidin, and or biotin. In various embodiments, the avidin, streptavidin, and/or biotin system may be used to associate cell markers with each other.

In various embodiments, biomarker-based standards may comprise markers simulating a set of markers for leukocytes. In various embodiments, the set of markers for leukocytes may comprise CD45. In various embodiments, a detection value from an analytical device may indicate CD45+. In various embodiments, CD45 may be conjugated to V500.

In various embodiments, biomarker-based standards may comprise markers simulating a set of markers for monocytes. In various embodiments, the set of markers for detecting monocytes may comprise CD45 and CD14. In various embodiments, a detection value for monocytes from an analytical device may indicate CD45+/CD14+. In various embodiments, CD45 may be conjugated to V500. In various embodiments, CD14 may be conjugated to perCP Cy5.5.

In various embodiments, biomarker-based standards may comprise markers simulating a set of markers for monocytes. In various embodiments, the set of markers for detecting NK cells may comprise CD45, CD3 and CD56. In various embodiments, a detection value for NK cells from an analytical device may indicate CD45+, CD3− and CD56+. In various embodiments, CD45 may be conjugated to V500. In various embodiments, CD3 may be conjugated to APC. In various embodiments, CD56 may be conjugated to PE.

In various embodiments, biomarker-based standards may comprise markers simulating a set of markers for monocytes. In various embodiments, the set of markers for detecting B cells may comprise CD45, CD3 and CD19. In various embodiments, a detection value for B cells from an analytical device may indicate CD45+, CD3− and CD19+. In various embodiments, CD45 may be conjugated to V500. In various embodiments, CD3 may be conjugated to APC. In various embodiments, CD19 may be conjugated to PE.Cy7.

In various embodiments, biomarker-based standards may comprise markers simulating a set of markers for early progenitor cells. In various embodiments, the set of markers for detecting early progenitor cells may comprise CD45, CD19 and CD10. In various embodiments, a detection value for early progenitor cells from an analytical device may indicate CD45dim, CD19+ and CD10+. In various embodiments, CD45 may be conjugated to V500. In various embodiments, CD19 may be conjugated to PE.Cy7. In various embodiments, CD10 may be conjugated to FITC.

In various embodiments, biomarker-based standards may comprise markers simulating a set of markers for stem cells. In various embodiments, the set of markers for detecting stem cells may comprise CD45, CD19 and CD34. In various embodiments, a detection value for stem cells from an analytical device may indicate CD45dim, CD19+ and CD34+. In various embodiments, CD45 may be conjugated to V500. In various embodiments, CD19 may be conjugated to PE.Cy7. In various embodiments, CD34 may be conjugated to BV421.

In various embodiments, all cells of a biological sample may undergo live/dead staining. In various embodiments, the fluorochrome may be 633/785 nm.

TABLE 20
Marker / Cell Type Association

| Marker Set | Cell Type |
|---|---|
| CD45+/CD14+ | Monocytes |
| CD45+/CD14−/CD3+ | T cells |
| CD45+/CD3−/CD56+ | NK cells |
| CD45+CD3−/CD19+ | B cells |
| CD45dim/CD19+/CD10+ | Early progenitor cells |
| CD45dim/CD19+/CD34+ | Stem cells |

Table 20 shows marker sets we developed. Using known computational methods, the overlapping markers may be deconvoluted. For example, CD45 may detect all leukocytes and using the marker sets may then be used to obtain relative values of different cell types (e.g., each impurity as a percentage of the whole leukocyte population). As can be seen in Table 20, many markers overlap, and only specific combinations may be used to determine a relative value for a given cell type. Once all values are obtained, they may be compared, statistically analyzed, and optionally corrected.

TABLE 21
Synthetic Positive Control Standard

| | | | Control |
|---|---|---|---|
| | | | Percent |
| BioMarker | Fluorochrome | Sub Population | Positive |
| CD45 | V500 | CD45+ | 50-60 |
| CD45dim | V500 | CD45dim | 40-50 |
| CD14 | PerCP Cy5.5 | CD45+/CD14+ | 25-30 |
| CD3 | APC | CD45+/CD14−/CD3+ | 40-50 |
| CD19 | PE.Cy7 | CD45/CD14−/CD3−/ | 25-30 |
| | | CD19+ | |
| CD56 | PE | CD45+/CD14−/CD56 | 25-30 |
| CD10 | FITC | CD45 dim/CD19+/CD10+ | 50-60 |
| CD34 | BV421 | CD45 | 50-60 |
| | | dim/CD19+/CD34+ | |
| CD45dim/CD19 | V500 | CD45 dim/CD19+ | 50-60 |
| LIVE/DEAD | 633/785 nm | CD45−/ Near IR+ | 15-20 |
| NEAR-IR | | | |

Table 21 shows BioMarkers in the first column, Fluorochromes, markers representing Sub Populations in the third column, and “Control Percent Positive” or Synthetic Cell Positive Control Expected Percent Positive. Note: The terms “synthetic cell positive control” and “biomarker-based standard(s)” may be used interchangeably.

Markers/marker sets and fluorochromes were extensively tested to eliminate design issues such as spectral overlap. For example, CD45 includes all leukocytes so an entire population should be included when CD45 and CD45dim are summed. in the second column.

A skilled artisan in the field of flow cytometry will appreciate a difference between CD45 or CD45hi and CD45dim include different and distinguishable values. The technology described herein may use CD45hi and CD45dim to distinguish between two groups of cell types. A first group includes early progenitor cells and stem cells. A second group includes monocytes, B cells, NK cells, NKT cells, and T cells. Additional markers described herein may be used to distinguish between the different cell types within the groups.

FIG. 9 graphically illustrates idealized processed data, displaying cell subpopulation relative values.

The top layer in FIG. 9 shows Live (85%) and Dead (15%). Our Live/Dead analysis was completed using “Live/Dead Fixable Near IR (876)™.” Live/Dead Fixable Near IR (876)™ is a commercially available viability dye used to determine live and dead cells in flow cytometry. For example, Thermo Fisher Scientific, Inc.™ offers Invitrogen™ Live/Dead Fixable Near IR (876)™ for sale in their current product catalog. Excitation lasers of 785 nm and 808 nm were used.

### Example 4: Comparison of Biomarker-Based Standards to Prior Art

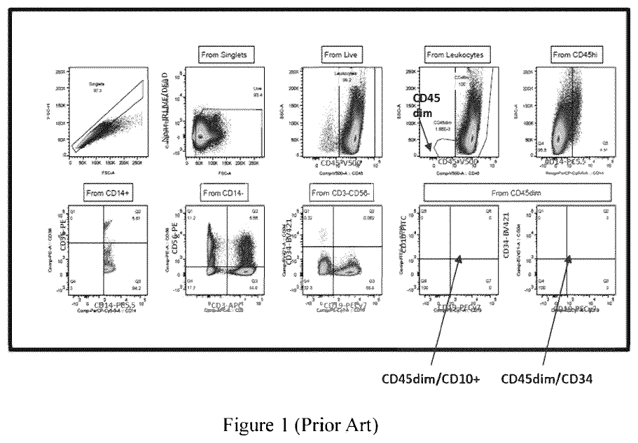

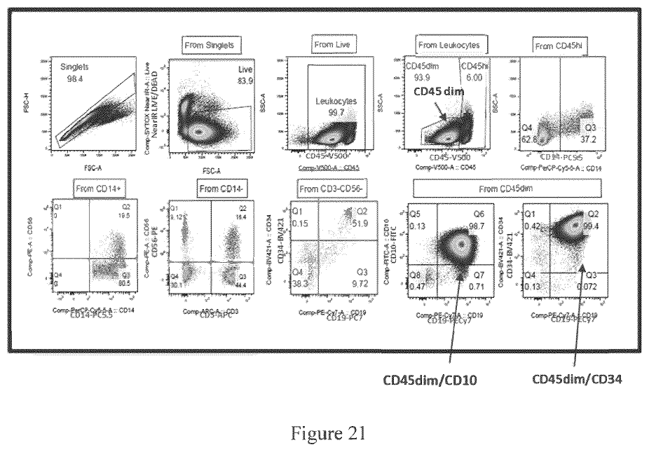

A biomarker-based standard was generated using live cell populations. FIG. 21 is experimental flow cytometry data using a biomarker-based standard. As seen in the bottom right two panels, CD10+ and CD34+ are present. Referring back to FIG. 1 (Prior Art), CD10+ and Cd34+ are lacking. As such, the newly developed biomarker standard performs as expected using the methods and concentrations described herein.

### CONCLUSIONS

Examples 1-4 demonstrate that biomarker-based standards outperform time-honored prior art positive controls in certain cell therapy processes. For example, synthetic positive controls comprise engineered marker sets that are unavailable in the prior art. Additionally, examples 1-4 also demonstrate compositions and methods that are more reliable over time than the prior art.

### RECITATION OF EMBODIMENTS

Embodiment 1: A composition for characterizing a population of cells into subpopulations, comprising a population of biomarker-based standards, wherein each biomarker-based standard comprises a common marker, wherein the common marker corresponds to a marker associated with leukocytes, at least one of a plurality of marker sets, wherein the marker sets correspond to marker sets of T cells, NK cells, monocytes, B cells, early progenitor cells, and stem cells, and a scaffold for restricting relative movement between the common marker and the at least one marker set.

Embodiment 2: The composition for characterizing a population of cells into subpopulations of embodiment 1, wherein the common marker is CD45, wherein CD45 is comprised of populations of CD45hi and CD45dim.

Embodiment 3: The composition for characterizing a population of cells into subpopulations according to any one of the preceding embodiments, wherein a marker set of the plurality of marker sets comprises CD10, CD19, CD45dim.

Embodiment 4: The composition for characterizing a population of cells into subpopulations according to any one of the preceding embodiments, wherein a marker set of the plurality of marker sets comprises CD19, CD34, and CD45dim.

Embodiment 5: The composition for characterizing a population of cells into subpopulations according to any one of the preceding embodiments, wherein a marker set of the plurality of marker sets comprises CD3. CD14, and CD45.

Embodiment 6: The composition for characterizing a population of cells into subpopulations according to any one of the preceding embodiments, wherein a marker set of the plurality of marker sets comprises CD3, CD45, and CD56.

Embodiment 7: The composition for characterizing a population of cells into subpopulations according to any one of the preceding embodiments, wherein a marker set of the plurality of marker sets comprises CD3, CD19, and CD45.

Embodiment 8: The composition for characterizing a population of cells into subpopulations according to any one of the preceding embodiments, wherein the plurality of marker sets comprise CD3, CD10, CD14, CD19, CD34, CD45, CD45dim, and CD56.

Embodiment 9: The composition for characterizing a population of cells into subpopulations according to any one of the preceding embodiments, wherein at least some of the population of biomarker-based standards further comprise amine binding sites that react to Near IR viability dye.

Embodiment 10: The composition for characterizing a population of cells into subpopulations of embodiment 9, wherein between about 15-20% of the population of biomarker-based standards comprise amine binding sites.

Embodiment 11: The composition for characterizing a population of cells into subpopulations of embodiment 9, wherein between about 16.7-100% of the population of biomarker-based standards do not comprise amine binding sites.

Embodiment 12: The composition for characterizing a population of cells into subpopulations according to any one of embodiments 2-11, wherein between about 50-60% of the population of biomarker-based standards comprise CD45hi.

Embodiment 13: The composition for characterizing a population of cells into subpopulations according to any one of embodiments 2-11, wherein between about 25.2-66.1% of the population of biomarker-based standards comprise CD45hi.

Embodiment 14: The composition for characterizing a population of cells into subpopulations according to any one of embodiments 2-11, wherein about 38.2% of the population of biomarker-based standards comprise CD45hi.

Embodiment 15: The composition for characterizing a population of cells into subpopulations according to any one of embodiments 2-14, wherein between about 40-50% of the population of biomarker-based standards comprise CD45dim.

Embodiment 16: The composition for characterizing a population of cells into subpopulations according to any one of embodiments 2-14, wherein between about 32.3-74.7% of the population of biomarker-based standards comprise CD45dim.

Embodiment 17: The composition for characterizing a population of cells into subpopulations according to any one of embodiments 2-14, wherein about 59.7% of the population of biomarker-based standards comprise CD45dim.

Embodiment 18: The composition for characterizing a population of cells into subpopulations according to any one of embodiments 3-17, wherein between about 50-60% of the population of biomarker-based standards comprise CD10, CD19, and CD45dim.

Embodiment 19: The composition for characterizing a population of cells into subpopulations according to any one of embodiments 3-17, wherein between about 29.7-66.7% of the population of biomarker-based standards comprise CD10, CD19, and CD45dim.

Embodiment 20: The composition for characterizing a population of cells into subpopulations according to any one of embodiments 3-17, wherein about 49.2 of the population of biomarker-based standards comprise CD10, CD19, and CD45dim.

Embodiment 21: The composition for characterizing a population of cells into subpopulations according to any one of embodiments 4-20, wherein between about 50-60% of the population of biomarker-based standards comprise CD19, CD34, and CD45dim.

Embodiment 22: The composition for characterizing a population of cells into subpopulations according to any one of embodiments 4-20, wherein between about 0.3-68.2% of the population of biomarker-based standards comprise CD19, CD34, and CD45dim.

Embodiment 23: The composition for characterizing a population of cells into subpopulations according to any one of embodiments 4-20, wherein about 45.6% of the population of biomarker-based standards comprise CD19. CD34, and CD45dim.

Embodiment 24: The composition for characterizing a population of cells into subpopulations according to any one of the preceding embodiments, wherein the markers are embedded within the scaffolds.

Embodiment 25: The composition for characterizing a population of cells into subpopulations according to any one of the preceding embodiments, wherein the markers are located on a surface of the scaffolds.

Embodiment 26: The composition for characterizing a population of cells into subpopulations according to any one of the preceding embodiments, wherein the scaffold comprises a phospholipid bilayer.

Embodiment 27: The composition for characterizing a population of cells into subpopulations according to any one of the preceding embodiments, wherein the scaffold comprises a hydrogel, an aerogel, a cryogel, an organogel, a nanogel, a conductive hydrogel, or any combination thereof.

Embodiment 28: A kit for characterizing a population of cells into subpopulations, comprising a population of biomarker-based standards, wherein each biomarker-based standards comprises a common marker, wherein the common marker corresponds to a marker associated with leukocytes, at least one of a plurality of marker sets, wherein the marker sets correspond to marker sets of T cells, NK cells, monocytes, B cells, early progenitor cells, and stem cells, and a scaffold for restricting relative movement between the common marker and the at least one marker set, and an antibody cocktail.

Embodiment 29: The kit for characterizing a population of cells into subpopulations of embodiment 28, wherein the common marker is CD45, wherein CD45 is comprised of populations of CD45hi and CD45dim.

Embodiment 30: The kit for characterizing a population of cells into subpopulations according to any one of embodiments 28-29, wherein a marker set of the plurality of marker sets comprises CD10, CD19, CD45dim.

Embodiment 31: The kit for characterizing a population of cells into subpopulations according to any one of embodiments 28-30, wherein a marker set of the plurality of marker sets comprises CD19, CD34, and CD45dim.

Embodiment 32: The kit for characterizing a population of cells into subpopulations according to any one of embodiments 28-31, wherein a marker set of the plurality of marker sets comprises CD3, CD14, and CD45.

Embodiment 33: The kit for characterizing a population of cells into subpopulations according to any one of embodiments 28-32, wherein a marker set of the plurality of marker sets comprises CD3, CD45, and CD56.

Embodiment 34: The kit for characterizing a population of cells into subpopulations according to any one of embodiments 28-33, wherein a marker set of the plurality of marker sets comprises CD3, CD19, and CD45.

Embodiment 35: The kit for characterizing a population of cells into subpopulations according to any one of embodiments 28-34, wherein the plurality of marker sets comprise CD3, CD10, CD14, CD19, CD34, CD45, CD45dim, and CD56.

Embodiment 36: The kit for characterizing a population of cells into subpopulations according to any one of embodiments 28-35, wherein at least some of the population of biomarker-based standards further comprise amine binding sites that react to Near IR viability dye.

Embodiment 37: The kit for characterizing a population of cells into subpopulations of embodiment 36, wherein between about 15-20% of the population of biomarker-based standards comprise amine binding sites.

Embodiment 38: The kit for characterizing a population of cells into subpopulations of embodiment 36, wherein between about 16.7-100% of the population of biomarker-based standards do not comprise amine binding sites.

Embodiment 39: The kit for characterizing a population of cells into subpopulations according to any one of embodiments 29-38, wherein between about 50-60% of the population of biomarker-based standards comprise CD45hi.

Embodiment 40: The kit for characterizing a population of cells into subpopulations according to any one of embodiments 29-38, wherein between about 25.2-66.1% of the population of biomarker-based standards comprise CD45hi.

Embodiment 41: The kit for characterizing a population of cells into subpopulations according to any one of embodiments 29-38, wherein about 38.2% of the population of biomarker-based standards comprise CD45hi.

Embodiment 42: The kit for characterizing a population of cells into subpopulations according to any one of embodiments 29-41, wherein between about 40-50% of the population of biomarker-based standards comprise CD45dim.

Embodiment 43: The kit for characterizing a population of cells into subpopulations according to any one of embodiments 29-42, wherein between about 32.3-74.7% of the population of biomarker-based standards comprise CD45dim.

Embodiment 44: The kit for characterizing a population of cells into subpopulations according to any one of embodiments 29-43, wherein about 59.7% of the population of biomarker-based standards comprise CD45dim.

Embodiment 45: The kit for characterizing a population of cells into subpopulations according to any one of embodiments 30-44, wherein between about 50-60% of the population of biomarker-based standards comprise CD10, CD19, and CD45dim.

Embodiment 46: The kit for characterizing a population of cells into subpopulations according to any one of embodiments 30-44, wherein between about 29.7-66.7% of the population of biomarker-based standards comprise CD10, CD19, and CD45dim.

Embodiment 47: The kit for characterizing a population of cells into subpopulations according to any one of embodiments 30-44, wherein about 49.2 of the population of biomarker-based standards comprise CD10, CD19, and CD45dim.

Embodiment 48: The kit for characterizing a population of cells into subpopulations according to any one of embodiments 30-47, wherein between about 50-60% of the population of biomarker-based standards comprise CD19, CD34, and CD45dim.

Embodiment 49: The kit for characterizing a population of cells into subpopulations according to any one of embodiments 30-47, wherein between about 0.3-68.2% of the population of biomarker-based standards comprise CD19, CD34, and CD45dim.

Embodiment 50: The kit for characterizing a population of cells into subpopulations according to any one of embodiments 30-47, wherein about 45.6% of the population of biomarker-based standards comprise CD19, CD34, and CD45dim.

Embodiment 51: The kit for characterizing a population of cells into subpopulations according to any one of embodiments 28-50, wherein the markers are embedded within the scaffolds.

Embodiment 52: The kit for characterizing a population of cells into subpopulations according to any one of embodiments 28-50, wherein the markers are located on a surface of the scaffolds.

Embodiment 53: The kit for characterizing a population of cells into subpopulations according to any one of embodiments 28-50, wherein the scaffold comprises a phospholipid bilayer.

Embodiment 54: The kit for characterizing a population of cells into subpopulations according to any one of embodiments 28-50, wherein the scaffold comprises a hydrogel, an aerogel, a cryogel, an organogel, a nanogel, a conductive hydrogel, or any combination thereof.

Embodiment 55: The kit for characterizing a population of cells into subpopulations according to any one of embodiments 28-54, wherein, wherein the antibody cocktail comprises an antibody for detecting the CD3, an antibody for detecting the CD10, an antibody for detecting the CD14, an antibody for detecting the CD19, an antibody for detecting the CD34, an antibody for detecting the CD45/CD45dim, and an antibody for detecting the CD56.

Embodiment 56: The kit for characterizing a population of cells into subpopulations of embodiment 55, wherein the antibody for detecting the CD3 is conjugated to an APC fluorochrome, the antibody for detecting the CD10 marker is conjugated to a FITC fluorochrome, the antibody for detecting the CD14 marker is conjugated to a PerCP Cy5.5 fluorochrome, the antibody for detecting the CD19 is conjugated to a PE.Cy7 fluorochrome, the antibody for detecting the CD34 is conjugated to a BV421 fluorochrome, the antibody for detecting the CD45/CD45dim marker is conjugated to a V500 fluorochrome, and the antibody for detecting the CD56 marker is conjugated to a PE fluorochrome.

Embodiment 57: A method for characterizing a population of cells into subpopulations, the method comprising providing a biological sample comprising a population of leukocytes, wherein the population of leukocytes comprises a plurality of subpopulations, wherein the subpopulations comprise T cells, NK cells, monocytes, B cells, B progenitor cells, and stem cells, providing a known quantity of a population of biomarker-based standards, wherein the biomarker-based standards comprise a common marker, wherein the common marker corresponds to a marker associated with leukocytes, at least one of a plurality of marker sets, wherein the marker sets correspond to marker sets of T cells, NK cells, monocytes, B cells, early progenitor cells, and stem cells, and a scaffold for restricting relative movement between the common marker and the at least one marker set, contacting the population of leukocytes and the population of biomarker-based standards with an antibody cocktail to create a mixture, analyzing the mixture with an analytical device, and characterizing the population of cells by subpopulation using the biomarker-based standards as a reference.

Embodiment 58: The method for characterizing a population of cells into subpopulations of embodiment 57, wherein the step of characterizing comprises determining a quantity of the population of leukocytes and quantities for each of the subpopulations.

Embodiment 59: The method for characterizing a population of cells into subpopulations of embodiment 57, wherein the step of characterizing comprises determining relative quantities or percentages of each of the subpopulations and the population of leukocytes.

Embodiment 60: The method for characterizing a population of cells into subpopulations of embodiment 57, wherein the common marker is CD45, wherein CD45 is comprised of populations of CD45hi and CD45dim.

Embodiment 61: The method for characterizing a population of cells into subpopulations according to any one of embodiments 57-60, wherein a marker set of the plurality of marker sets comprises CD10, CD19, CD45dim.

Embodiment 62: The method for characterizing a population of cells into subpopulations according to any one of embodiments 57-61, wherein a marker set of the plurality of marker sets comprises CD19, CD34, and CD45dim.

Embodiment 63: The method for characterizing a population of cells into subpopulations according to any one of embodiments 57-62, wherein a marker set of the plurality of marker sets comprises CD3, CD14, and CD45.

Embodiment 64: The method for characterizing a population of cells into subpopulations according to any one of embodiments 57-63, wherein a marker set of the plurality of marker sets comprises CD3, CD45, and CD56.

Embodiment 65: The method for characterizing a population of cells into subpopulations according to any one of embodiments 57-64, wherein a marker set of the plurality of marker sets comprises CD3, CD19, and CD45.

Embodiment 66: The method for characterizing a population of cells into subpopulations according to any one of embodiments 57-65, wherein the plurality of marker sets comprise CD3, CD10, CD14, CD19, CD34, CD45, CD45dim, and CD56.

Embodiment 67: The method for characterizing a population of cells into subpopulations according to any one of embodiments 57-66, wherein at least some of the population of biomarker-based standards further comprise amine binding sites that react to Near IR viability dye.

Embodiment 68: The method for characterizing a population of cells into subpopulations of embodiment 67, wherein between about 15-20% of the population of biomarker-based standards comprise amine binding sites.

Embodiment 69: The method for characterizing a population of cells into subpopulations of embodiment 67, wherein between about 16.7-100% of the population of biomarker-based standards do not comprise amine binding sites.

Embodiment 70: The method for characterizing a population of cells into subpopulations according to any one of embodiments 60-69, wherein between about 50-60% of the population of biomarker-based standards comprise CD45hi.

Embodiment 71: The method for characterizing a population of cells into subpopulations according to any one of embodiments 60-69, wherein between about 25.2-66.1% of the population of biomarker-based standards comprise CD45hi.

Embodiment 72: The method for characterizing a population of cells into subpopulations according to any one of embodiments 60-69, wherein about 38.2% of the population of biomarker-based standards comprise CD45hi.

Embodiment 73: The method for characterizing a population of cells into subpopulations according to any one of embodiments 60-72, wherein between about 40-50% of the population of biomarker-based standards comprise CD45dim.

Embodiment 74: The method for characterizing a population of cells into subpopulations according to any one of embodiments 60-72, wherein between about 32.3-74.7% of the population of biomarker-based standards comprise CD45dim.

Embodiment 75: The method for characterizing a population of cells into subpopulations according to any one of embodiments 60-72, wherein about 59.7% of the population of biomarker-based standards comprise CD45dim.

Embodiment 76: The method for characterizing a population of cells into subpopulations according to any one of embodiments 61-75, wherein between about 50-60% of the population of biomarker-based standards comprise CD10, CD19, and CD45dim.

Embodiment 77: The method for characterizing a population of cells into subpopulations according to any one of embodiments 61-75, wherein between about 29.7-66.7% of the population of biomarker-based standards comprise CD10, CD19, and CD45dim.

Embodiment 78: The method for characterizing a population of cells into subpopulations according to any one of embodiments 61-75, wherein about 49.2 of the population of biomarker-based standards comprise CD10, CD19, and CD45dim.

Embodiment 79: The method for characterizing a population of cells into subpopulations according to any one of embodiments 62-78, wherein between about 50-60% of the population of biomarker-based standards comprise CD19, CD34, and CD45dim.

Embodiment 80: The method for characterizing a population of cells into subpopulations according to any one of embodiments 62-78, wherein between about 0.3-68.2% of the population of biomarker-based standards comprise CD19, CD34, and CD45dim.

Embodiment 81: The method for characterizing a population of cells into subpopulations according to any one of embodiments 62-78, wherein about 45.6% of the population of biomarker-based standards comprise CD19, CD34, and CD45dim.

Embodiment 82: The method for characterizing a population of cells into subpopulations according to any one of embodiments 57-80, wherein the markers are embedded within the scaffolds.

Embodiment 83: The method for characterizing a population of cells into subpopulations according to any one of embodiments 57-80, wherein the markers are located on a surface of the scaffolds.

Embodiment 84: The method for characterizing a population of cells into subpopulations according to any one of embodiments 57-80, wherein the scaffold comprises a phospholipid bilayer.

Embodiment 85: The method for characterizing a population of cells into subpopulations according to any one of embodiments 57-80, wherein the scaffold comprises a hydrogel, an aerogel, a cryogel, an organogel, a nanogel, a conductive hydrogel, or any combination thereof.

Embodiment 86: The method for characterizing a population of cells into subpopulations according to any one of embodiments 57-84, further comprising an antibody cocktail, wherein the antibody cocktail comprises an antibody for detecting the CD3, an antibody for detecting the CD10, an antibody for detecting the CD14, an antibody for detecting the CD19, an antibody for detecting the CD34, an antibody for detecting the CD45/CD45dim, and an antibody for detecting the CD56.

Embodiment 87: The method for characterizing a population of cells into subpopulations of embodiment 85, wherein the antibody for detecting the CD3 is conjugated to an APC fluorochrome, the antibody for detecting the CD10 marker is conjugated to a FITC fluorochrome, the antibody for detecting the CD14 marker is conjugated to a PerCP Cy5.5 fluorochrome, the antibody for detecting the CD19 is conjugated to a PE.Cy7 fluorochrome, the antibody for detecting the CD34 is conjugated to a BV421 fluorochrome, the antibody for detecting the CD45/CD45dim marker is conjugated to a V500 fluorochrome, and the antibody for detecting the CD56 marker is conjugated to a PE fluorochrome.

Embodiment 88: The method for characterizing a population of cells into subpopulations according to any one of embodiments 57-86, wherein the biological sample comprises an apheresis.

Embodiment 89: The method for characterizing a population of cells into subpopulations according to any one of embodiments 57-86, wherein the biological sample comprises a final product.

Embodiment 90: The method for characterizing a population of cells into subpopulations according to any one of embodiments 57-86, wherein the analytical device comprises a flow cytometer, wherein the flow cytometer comprises a plurality of lasers directed through a flow channel for exciting the fluorochromes and at least one detector.

Embodiment 91: The method for characterizing a population of cells into subpopulations of embodiment 89, wherein the step of analyzing further comprises flowing the mixture through the flow cell, interrogating each of the cells in the populations of leukocytes and each of the biomarker-based standards with each of the plurality of lasers, and capturing signal intensity data for each marker.

### EQUIVALENCE

Those skilled in the art will recognize or be able to ascertain using no more than routine experimentation, many equivalents to the specified embodiments of the technologies described herein. It is to be understood that the technologies encompass all variants, combinations, and permutations in which one or more limitations, elements, clauses, descriptive terms, etc., from one or more of the listed claims is introduced into another claim dependent on the same base claim (or, as relevant, any other claim) unless otherwise indicated or unless it would be evident to one of ordinary skill in the art that a contradiction or inconsistency would arise. Further, it should also be understood that any embodiment or aspect of the technologies can be explicitly excluded from the claims, regardless of whether the specific exclusion is recited in the specification.

1. A composition for characterizing a population of cells into subpopulations, comprising:
a population of biomarker-based standards, wherein each biomarker-based standard comprises:
a common marker, wherein the common marker corresponds to a marker associated with leukocytes;
at least one of a plurality of marker sets, wherein the marker sets correspond to marker sets of T cells, NK cells, monocytes, B cells, early progenitor cells, and stem cells; and
a scaffold for restricting relative movement between the common marker and the at least one marker set.

2. The composition for characterizing a population of cells into subpopulations of claim 1, wherein the common marker is CD45, wherein CD45 is comprised of populations of CD45hi and CD45dim.

3. The composition for characterizing a population of cells into subpopulations of claim 1, wherein a marker set of the plurality of marker sets comprises CD10, CD19, CD45dim.

4. The composition for characterizing a population of cells into subpopulations of claim 1, wherein a marker set of the plurality of marker sets comprises CD19, CD34, and CD45dim.

5. The composition for characterizing a population of cells into subpopulations of claim 1, wherein a marker set of the plurality of marker sets comprises CD3, CD14, and CD45.

6. The composition for characterizing a population of cells into subpopulations of claim 1, wherein a marker set of the plurality of marker sets comprises CD3, CD45, and CD56.

7. The composition for characterizing a population of cells into subpopulations of claim 1, wherein a marker set of the plurality of marker sets comprises CD3, CD19, and CD45.

8. The composition for characterizing a population of cells into subpopulations of claim 1, wherein the plurality of marker sets comprise CD3, CD10, CD14, CD19, CD34, CD45, CD45dim, and CD56.

9. The composition for characterizing a population of cells into subpopulations of claim 1, wherein at least some of the population of biomarker-based standards further comprise amine binding sites that react to Near IR viability dye.

10. The composition for characterizing a population of cells into subpopulations of claim 9, wherein between about 15-20% of the population of biomarker-based standards comprise amine binding sites.

11. The composition for characterizing a population of cells into subpopulations of claim 9, wherein between about 16.7-100% of the population of biomarker-based standards do not comprise amine binding sites.

12. The composition for characterizing a population of cells into subpopulations of claim 2, wherein between about 50-60% of the population of biomarker-based standards comprise CD45hi.

13. The composition for characterizing a population of cells into subpopulations of claim 2, wherein between about 25.2-66.1% of the population of biomarker-based standards comprise CD45hi.

14. The composition for characterizing a population of cells into subpopulations of claim 2, wherein about 38.2% of the population of biomarker-based standards comprise CD45hi.

15. The composition for characterizing a population of cells into subpopulations of claim 2, wherein between about 40-50% of the population of biomarker-based standards comprise CD45dim.

16. The composition for characterizing a population of cells into subpopulations of claim 2, wherein between about 32.3-74.7% of the population of biomarker-based standards comprise CD45dim.

17. The composition for characterizing a population of cells into subpopulations of claim 2, wherein about 59.7% of the population of biomarker-based standards comprise CD45dim.

18. The composition for characterizing a population of cells into subpopulations of claim 2,
wherein between about 50-60%, or about 29.7-66.7%, or about 49.2% of the population of biomarker-based standards comprise CD10, CD19, and CD45dim; or
wherein between about 50-60%, about 0.3-68.2%, or about 45.6% of the population of biomarker-based standards comprise CD19, CD34, and CD45dim.

19-27. (canceled)

28. A kit for characterizing a population of cells into subpopulations, comprising:
a population of biomarker-based standards, wherein each biomarker-based standards comprises:
a common marker, wherein the common marker corresponds to a marker associated with leukocytes;
at least one of a plurality of marker sets, wherein the marker sets correspond to marker sets of T cells, NK cells, monocytes, B cells, early progenitor cells, and stem cells; and
a scaffold for restricting relative movement between the common marker and the at least one marker set; and
an antibody cocktail.

29. A method for characterizing a population of cells into subpopulations, the method comprising:
providing a biological sample comprising a population of leukocytes, wherein the population of leukocytes comprises a plurality of subpopulations, wherein the subpopulations comprise T cells, NK cells, monocytes, B cells, B progenitor cells, and stem cells;
providing a known quantity of a population of biomarker-based standards, wherein the biomarker-based standards comprise:
a common marker, wherein the common marker corresponds to a marker associated with leukocytes;
at least one of a plurality of marker sets, wherein the marker sets correspond to marker sets of T cells, NK cells, monocytes, B cells, early progenitor cells, and stem cells; and
a scaffold for restricting relative movement between the common marker and the at least one marker set;
contacting the population of leukocytes and the population of biomarker-based standards with an antibody cocktail to create a mixture;
analyzing the mixture with an analytical device; and
characterizing the population of cells by subpopulation using the biomarker-based standards as a reference.
